(19) United States
(12) Patent Application Publication
(10) Pub. No.: US 2026/0003434 A1
(43) Pub. Date: Jan. 1, 2026

(54) CONTROL OF COMPUTER OPERATIONS VIA TRANSLATION OF BIOLOGICAL SIGNALS AND TRAUMATIC BRAIN INJURY PREDICTION BASED ON SLEEP STATES

(71) Applicant: NEUROVIGIL, INC., Moffett Field, CA (US)

(72) Inventor: Philip LOW, Moffett Field, CA (US)

(73) Assignee: NEUROVIGIL, INC., Moffett Field, CA (US)

(21) Appl. No.: 19/318,966

(22) Filed: Sep. 4, 2025

Related U.S. Application Data
(63) Continuation of application No. PCT/US2024/019891, filed on Mar. 14, 2024.
(60) Provisional application No. 63/452,268, filed on Mar. 15, 2023.
(60) Provisional application No. 63/452,275, filed on Mar. 15, 2023.

(51) Int. Cl. G06F 3/01; A61B 5/00; A61B 5/372; A61B 5/397; G06F 3/0481

(52) U.S. Cl. CPC G06F 3/015; A61B 5/372; A61B 5/397; A61B 5/4812; G06F 3/0481

(57) ABSTRACT

Method and systems for translating biological signals to perform various operations associated with a computing device is provided. The method can include accessing biological-signal data that was collected by a biological-signal data acquisition assembly that comprises a housing having one or more clusters of electrodes. Each cluster of the one or more clusters of electrodes can include at least an active electrode. The method can also include identifying, based on the biological-signal data, a first signal that represents a first intent to move a first portion of a body of the subject. The first signal is generated before a second signal, in which the second signal represents a second intent to move a second portion of the body of the subject. The method can also include translating the first signal to identify a first operation to be performed by a computing device.

### CROSS-REFERENCES TO RELATED APPLICATIONS

This application is a continuation of International Patent Application No. PCT/US2024/019891, filed on Mar. 14, 2024, which claims the benefit of U.S. Provisional Application No. 63/452,268, filed on Mar. 15, 2023, and entitled “CONTROL OF COMPUTER OPERATIONS VIA TRANSLATION OF BIOLOGICAL SIGNALS”, and U.S. provisional Application No. 63/452,275, filed on Mar. 15, 2023, and entitled “TRAUMATIC BRAIN INJURY PREDICTION BASED ON SLEEP STATES”, the entirety of each of which is hereby incorporated by reference herein.

### FIELD OF INVENTION

The present disclosure relates generally to translating biological signals from a subject to identify operations to be performed by a computing device. Specifically, the present disclosure relates to methods and system for analyzing activation sequence of biological signals to identify one or more operations to be performed by a computing device. The present disclosure further relates generally to analyzing physiological data and, more particularly (although not necessarily exclusively), to predicting the presence of a traumatic brain injury based on metrics associated with sleep states.

### BACKGROUND

### Control of Computer Operations Via Translation of Biological Signals

Various neurons in the brain cooperate to generate a rich and continuous set of neural electrical signals. Such signals have powerful influence on the control of the body. For example, the signals can initiate body movements and facilitate cognitive thoughts. In addition, neural signals can cause humans to wake during sleep. A deeper understanding of the signal-to-action biological pathway can provide a potential for using biological signals to perform actions previously unavailable to humans (e.g., using thoughts to move a mouse cursor).

Brain-computer interfaces (BCI) can be configured to translate the brain's electrical activity to determine operations performed by an external device. For example, biological signals from the brain can be analyzed to control a cursor or manipulate prosthetic devices. Thus, BCIs are often used for researching, mapping, assisting, augmenting, or repairing human cognitive or sensory-motor functions. Implementations of BCIs range from non-invasive (EEG, MEG, EOG, MRI), partially invasive (ECoG and endovascular), and invasive procedures (microelectrode array), in which the invasiveness of the procedures is based on how close electrodes are positioned relative to the brain tissue.

Despite decades of intense research, a direct translation from the brain signals to various human actions remains challenging due to the complexity of the signals. In addition, directly translating the brain signals to identify certain tasks can be inefficient because the translation would necessitate several signal preprocessing steps to be performed before the signals are actually translated. For example, translating the brain signals to write English sentences may require removal of noise in the brain signals that are irrelevant to the task in hand. Such difficulties can also occur for translating the brain signals to human actions that relate to controlling of another device. Accordingly, there is a need for techniques that are capable of efficiently translating biological signals to perform various computing-device operations.

### Traumatic Brain Injury Prediction Based on Sleep States

An electroencephalogram (EEG) is a tool used to measure electrical activity produced by the brain. The functional activity of the brain is collected by electrodes placed on the scalp of a subject. Conventional monitoring and diagnostic equipment includes several electrodes mounted on the subject, which tap the brain signals and transmit the signals via cables to amplifier units. The EEG signals obtained can be used to diagnose and monitor various conditions that affect the brain.

For example, traumatic brain injuries (TBIs) can occur when normal functioning of the brain is disrupted by an external force (e.g., impact to the head, sudden acceleration or deceleration, penetrating head injury, etc.) experienced by a subject. TBIs can be classified as mild, moderate, or severe based on a severity of the disruption to normal brain function at the time the subject experienced the external force. Symptoms for mild TBIs (i.e., concussions) can include headache, dizziness, impaired vision, sensitivity to light, behavioral changes, etc. Symptoms for moderate to severe TBIs can include the above symptoms and can additionally include slurred speech, nausea, seizures, loss of consciousness, etc.

However, it can be difficult to detect and diagnose TBIs. For example, diagnosis of TBIs can include performing a neurological exam on the subject, which can evaluate the above symptoms as well as thinking, motor function, coordination, sensory function, reflexes, etc. It can be difficult to determine whether the subject has a TBI from the neurological exam due to normal or average motor function, coordination, etc. being different for and specific to each subject. Thus, the neurological exam can be an ineffective method of determining whether the subject is experiencing changes in thinking, motor function, coordination, etc., thereby rendering it ineffective for diagnosing TBIs. The neurological exam can be especially ineffective in cases where the symptoms are subtle (e.g., mild TBIs) and/or in cases where baseline information (i.e., normal thinking, motor function, coordination, etc.) for the subject is unknown. Moreover, there are currently no FDA approved medical devices intended to be used alone in diagnosing TBIs. For example, imaging modalities (e.g., CT scans, MRI scans, etc.) are often unable to show signs of traumatic brain injury. In particular, the imaging modalities may detect bleeding or other suitable signs of moderate or severe TBIs, but the imaging modalities may not detect signs of mild TBIs. Therefore, there can be a need for a more reliable technique for detecting and diagnosing TBIs.

### SUMMARY

In some embodiments, a method of translating biological signals to perform various operations associated with a computing device is provided. The method can include accessing biological-signal data that was collected by a biological-signal data acquisition assembly that comprises a housing having one or more clusters of electrodes. Each cluster of the one or more clusters of electrodes can include at least an active electrode. The method can also include identifying, based on the biological-signal data, a first signal that represents a first intent to move a first portion of a body of the subject. The first signal is generated before a second signal, in which the second signal represents a second intent to move a second portion of the body of the subject. The method can also include translating the first signal to identify a first operation to be performed by a computing device. The method can also include outputting first instructions to perform the first operation.

In some embodiments, the biological-signal data includes electroencephalography (EEG) data, in which the first signal is generated from a left hemisphere of a brain of the subject and the second signal is generated from a right hemisphere of the brain. In some embodiments, the biological-signal data includes electromyography (EMG) data, in which the first portion is a left limb of the subject and the second portion is a right limb of the subject.

The first operation can include performing one or more functions associated with a graphical user interface of the computing device. For example, the first operation can include moving a cursor displayed on the graphical user interface from a first location to a second location. In another example, the first operation can include inputting text onto the graphical user interface. After the text is inputted onto the graphical user interface, one or more machine-learning models can be applied to the inputted text to predict additional text to be inputted onto the graphical user interface. In yet another example, the first operation can also include inputting one or more images or icons on the graphical user interface. In some embodiments, the first operation includes launching an application stored in the computing device or executing one or more commands associated with the application.

In some embodiments, the first operation includes accessing one or more interface elements of an intent-communication interface to identify one or more operations to be performed by the computing device. In some embodiments, the intent-communication interface is a tree that includes a root interface element connected to the first interface element and the second interface element.

Accessing the interface elements can include selecting a first interface element over a second interface element of an intent-communication interface. The first interface element is associated with a first interface-operation data and a second interface element is associated with a second interface-operation data. A second operation to be performed by the computing device is identified based on the first interface-operation data. Second instructions to perform the second operation can be outputted.

Other interface elements of the intent-communication interface can be accessed based on biological signals collected at different time points. Additional biological-signal data that was collected by the biological-signal data acquisition assembly can be accessed at another time point. Based on the additional biological-signal data, a third signal that represents a third intent to move the second portion of a body of the subject can be identified. In some embodiments, the third signal is generated before a fourth signal, in which the fourth signal represents a fourth intent to move the first portion of the body of the subject. The third signal can be translated to identify a third operation to be performed by a computing device. Based on the third operation, a third interface element can be selected over a fourth interface element of the intent-communication interface, in which the third interface element is associated with a third interface-operation data and a fourth interface element is associated with a fourth interface-operation data. The third interface element and the fourth interface element are connected to the first interface element. A fourth operation to be performed by the computing device can be identified by accessing the third interface-operation data of the selected third interface element. In some embodiments, the fourth operation includes inputting one or more alphanumerical characters on a graphical user interface of the computing device. Third instructions to perform the fourth operation can then be outputted.

Additionally or alternatively, the first operation can be used to control various types of devices. For example, the computing device can be an augmented reality or virtual reality device, and the first operation can include performing one or more operations associated with the augmented reality or virtual reality device. In another example, the computing device can include one or more robotic components, in which the first operation includes controlling the one or more robotic components.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Some embodiments relate to a computer-implemented method. The method includes accessing neural-signal data indicative of electrical activity from a part of the brain of a subject over one or more sleep time periods, predicting a segment-specific metric associated with a sleep stage for each of one or more time segments in the one or more sleep time periods, generating a cumulative metric based on the segment-specific metrics, generating a risk-level metric for the subject based on the cumulative metric, and outputting a result that is based on or that represents the cumulative metric.

In some embodiments, the cumulative metric corresponds to an estimated absolute or relative time during which the subject was in a Stage 2 sleep stage. In some embodiments, the risk-level metric represents a likelihood that the subject has a traumatic brain injury.

In some embodiments, predicting the segment-specific metric includes performing at least one Fourier transform on the neural signal data in the segment. In some embodiments, the method includes determining that an alert condition is satisfied based on the cumulative metric. In some embodiments, the result is output in response to determining that the alert condition is satisfied.

In some embodiments, outputting the result includes transmitting an alert communication to a third-party system associated with monitoring the subject. In some embodiments, the neural-signal data includes electroencephalography data. In some embodiments, the segment-specific metric identifies a predicted sleep stage. In some embodiments, the segment-specific metric identifies a predicted probability of the subject being in the Stage 2 sleep stage.

Some embodiments relate to a system. The system includes one or more data processors, and a non-transitory computer readable storage medium containing instructions. When executed on the one or more data processors, the instructions cause the one or more data processors to access neural-signal data indicative of electrical activity from a part of the brain of a subject over one or more sleep time periods, predict a segment-specific metric associated with a sleep stage for each of one or more time segments in the one or more sleep time periods, generate a cumulative metric based on the segment-specific metrics, generate a risk-level metric for the subject based on the cumulative metric, and output a result that is based on or that represents the cumulative metric.

In some embodiments, the cumulative metric corresponds to an estimated absolute or relative time during which the subject was in a Stage 2 sleep stage. In some embodiments, the risk-level metric represents a likelihood that the subject has a traumatic brain injury.

In some embodiments, predicting the segment-specific metric includes performing at least one Fourier transform on the neural signal data in the segment. In some embodiments, the instructions when executed on the one or more data processors cause the one or more data processors to further determine that an alert condition is satisfied based on the cumulative metric. In some embodiments, the result is output in response to determining that the alert condition is satisfied.

In some embodiments, outputting the result includes transmitting an alert communication to a third-party system associated with monitoring the subject. In some embodiments, the neural-signal data includes electroencephalography data. In some embodiments, the segment-specific metric identifies a predicted sleep stage. In some embodiments, the segment-specific metric identifies a predicted probability of the subject being in the Stage 2 sleep stage

Some embodiments relate to a computer-program product tangibly embodied in a non-transitory machine-readable storage medium, including instructions. The instructions cause one or more data processors to access neural-signal data indicative of electrical activity from a part of the brain of a subject over one or more sleep time periods, predict a segment-specific metric associated with a sleep stage for each of one or more time segments in the one or more sleep time periods, generate a cumulative metric based on the segment-specific metrics, generate a risk-level metric for the subject based on the cumulative metric, and output a result that is based on or that represents the cumulative metric.

In some embodiments, the cumulative metric corresponds to an estimated absolute or relative time during which the subject was in a Stage 2 sleep stage. In some embodiments, the risk-level metric represents a likelihood that the subject has a traumatic brain injury.

In some embodiments, predicting the segment-specific metric includes performing at least one Fourier transform on the neural signal data in the segment. In some embodiments, the instructions cause the one or more data processors to further determine that an alert condition is satisfied based on the cumulative metric. In some embodiments, the result is output in response to determining that the alert condition is satisfied.

In some embodiments, outputting the result includes transmitting an alert communication to a third-party system associated with monitoring the subject. In some embodiments, the neural-signal data includes electroencephalography data. In some embodiments, the segment-specific metric identifies a predicted sleep stage. In some embodiments, the segment-specific metric identifies a predicted probability of the subject being in the Stage 2 sleep stage.

The terms and expressions which have been employed are used as terms of description and not of limitation, and there is no intention in the use of such terms and expressions of excluding any equivalents of the features shown and described or portions thereof, but it is recognized that various modifications are possible within the scope of the present disclosure. Thus, it should be understood that although the present, as claimed, has been specifically disclosed by some embodiments and optional features, modification and variation of the concepts herein disclosed may be resorted to by those skilled in the art, and that such modifications and variations are considered to be within the scope as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a user wearing a multi-electrode compact device that is wirelessly communicating with another electronic device.

FIG. 2 shows one embodiment of devices connected on a network to facilitate coordinated assessment and use of biological electrical recordings.

FIG. 3 shows one embodiment of a multi-electrode device communicating wirelessly with another electronic device.

FIG. 4 is a simplified block diagram of one embodiment of a multi-electrode device.

FIG. 5 is a simplified block diagram of one embodiment of an electronic device in communication with a multi-electrode device.

FIG. 6 is a flow diagram of one embodiment of a process for using a multi-electrode device to collect a channel of biological electrode data.

FIG. 7 is a flow diagram of one embodiment of a process for analyzing channel biological data to identify frequency signatures of various biological stages.

FIG. 8 is a flow diagram of one embodiment of a process for analyzing channel biological data to identify frequency signatures of various biological stages.

FIG. 9 is a flow diagram of one embodiment of a process for normalizing a spectrogram and using a group-distinguishing frequency signature to classify biological data.

FIG. 10 illustrates a schematic diagram that shows an example of determining an activation sequence of biological signals, according to some embodiments.

FIG. 11 illustrates an example of an intent-communication interface used for translating biological-signal data to one or more computing-device operations, according to some embodiments.

FIG. 12 illustrates a process for translating biological-signal data to one or more computing-device operations, in accordance with some embodiments.

FIG. 13 illustrates an example schematic diagram of using an intent-communication interface for inputting text and images, according to some embodiments.

FIG. 14 depicts an example of an intent-communication interface for inputting images, according to some embodiments.

FIG. 15 depicts another example of an intent-communication interface for inputting text of other languages, according to some embodiments.

FIG. 16 depicts an example of an intent-communication interface for operating a computer application.

FIG. 17 depicts a schematic diagram of using machine-learning techniques to enhance an intent-communication interface, according to some embodiments.

FIG. 18 depicts an example operation of the recurrent neural network for generating predicted words based on text data, according to some embodiments.

FIG. 19 illustrates another example of a recurrent neural network operation for generating predicted words based on text data, according to some embodiments.

FIG. 20 depicts an example schematic diagram of a long short-term memory network for generating predicted words based on text data, according to some embodiments.

FIG. 21 illustrates an example schematic diagram for implementing forget and input gates of a long short-term memory network, according to some embodiments.

FIG. 22 depicts an example operation of an output gate of a long short-term memory network, according to some embodiments.

FIG. 23 illustrates an example schematic diagram of using an intent-communication interface for translating biological-signal data to one or more operations associated with a virtual-reality device, according to some embodiments.

FIG. 24 illustrates an example schematic diagram of using an intent-communication interface for translating biological-signal data to one or more operations associated with a computing device with one or more robotic components, according to some embodiments.

FIG. 25 illustrates an example schematic diagram of using an intent-communication interface for translating biological-signal data to one or more operations associated with an accessory device, according to some embodiments.

FIG. 26 depicts a computing system that can implement any of the computing systems or environments discussed above.

FIG. 27 is a block diagram of an example of a system for acquiring physiological data according to one example of the present disclosure.

FIG. 28 is an example of a graph for predicting stage two sleep according to one example of the present disclosure.

FIG. 29 is a block diagram of an example of a system for predicting the presence of a traumatic brain injury based on metrics associated with sleep states according to one example of the present disclosure.

FIG. 30 is a block diagram of an example of a computing system for predicting the presence of a traumatic brain injury based on metrics associated with sleep states according to one example of the present disclosure.

FIG. 31 is a flowchart of a process for predicting the presence of a traumatic brain injury based on metrics associated with sleep states according to one example of the present disclosure.

### DETAILED DESCRIPTION

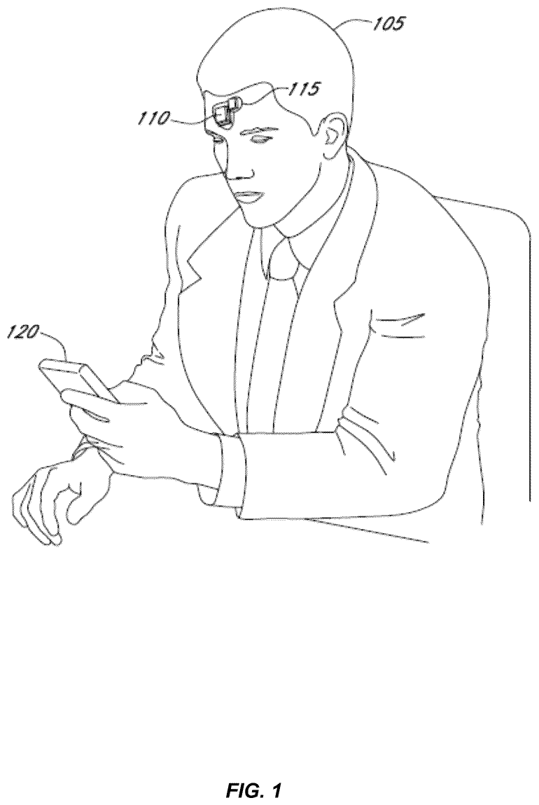

Certain embodiments disclosed herein can facilitate translation of biological signals (e.g., electroencephalography (EEG) data, electromyography (EMG) data) to identify various operations associated with a computing device. In some embodiments, a signal-processing application accesses biological-signal data of a subject. In some instances, the biological-signal data are collected by a biological-signal data acquisition assembly. The biological-signal data acquisition assembly (e.g., a multi-electrode device 110 of FIG. 1) can include a housing having one or more clusters of electrodes, in which each cluster of the one or more clusters of electrodes includes at least an active electrode. The biological-signal data collected by the biological-signal data acquisition assembly can include different types of biological signals. For example, the biological-signal data can include EEG data collected from electrodes placed on the subject's forehead. In another example, the biological-signal data can include EMG data collected from electrodes placed on the subject's limbs. In some instances, the biological-signal data are accessed via a wireless communication network (e.g., a short-range communication network).

The biological signals from the subject can be analyzed by the signal-processing application to detect a signal-activation sequence. For example, detecting the signal-activation sequence can include processing the biological-signal data to identify a first signal that represents a first intent to move a first portion of the body of the subject, in which the first signal was generated before a second signal. In some instances, the second signal represents a second intent to move a second portion of the body of the subject. For example, if the biological-signal data include EEG data, the first signal is generated from a left hemisphere of the brain, which was generated before the second signal that was generated from a right hemisphere of the brain of the subject. In another example, if the biological-signal data include EMG data, the biological-signal data can be analyzed to detect that the first signal representing an intent to move a first muscle (e.g., a left arm) was generated before the second signal representing another intent to move a second muscle (e.g., a right arm) of the subject. Additionally or alternatively, both EEG and EMG data can be used to determine that the first signal was generated before the second signal.

Based on the first signal being generated before the second signal, the signal-processing application identifies a particular operation to be performed by a computing device. The operation may include inputting one or more alphanumerical characters on a graphical user interface of the computing device. In another example, the operation can include moving a cursor displayed by the graphical user interface. The operations can also include operations that are performed by different types of computing devices, including controlling one or more robotic components or controlling augmented reality or virtual reality devices. The signal-processing application can then output instructions for the computing device to perform the identified operation. In some instances, the signal-processing application is internal to the computing device, in which the computing device can directly access the instructions and perform the operation. In some embodiments, the signal-processing application is external to the computing device. For example, the signal-processing application can be a part of an interface system (e.g., a BCI system), in which the signal-processing application can transmit, over a communication network, the instructions to the computing device to perform the operation. Additionally or alternatively, the signal-processing application can transmit instructions to one or more accessory devices (e.g., smartwatch) communicatively coupled to the computing device, such that the one or more accessory devices can perform the identified operation.

In some embodiments, the identified operation includes accessing interface-operation data from one or more intent-communication interfaces, in which the interface-operation data is used to determine another operation to be performed by the computing device. In some instances, an intent-communication interface includes a set of interface elements, in which at least one interface element of the set includes a corresponding interface-operation data. As an illustrative example, a tree including a plurality of nodes can be accessed, in which each node of the plurality of nodes of the tree is connected with one or more children nodes. Each interface element can include interface-operation data that identifies the particular operation, which can be accessed when the biological-signal data indicates that left and right portions of the body have been simultaneously activated (e.g., both portions activated within a predetermined time interval). The interface-operation data can be used by the same or another computing device to perform the particular operation. For example, an interface element can include interface-operation data corresponding to a “z” alphabetical character, and the identified operation to be performed by the computing device includes inputting the “z” character into a graphical user interface associated with the computing device.

In some instances, activation sequences of biological signals across a plurality of times are used to traverse one or more interface elements of the intent-communication interface, until a particular interface element is accessed and an associated operation is accessed. As an illustrative example, a user-interface operation can initiate from a root interface element of the intent-communication interface. At a first time point, biological signals detected from the subject can be processed to determine that a first signal that represents an intent to move a first portion of the body (e.g., an intent to squeeze a left hand) was generated before a second signal that represents another intent to move a second portion of the body (e.g., an intent to squeeze a right hand). Based on such determination, a left child interface element connected to the root interface element can be accessed. If it is determined that the left child interface element includes two children interface elements, the traversal of the intent-communication interface can continue with the left child interface element. Then, biological signals detected from the subject at a second time point can be analyzed to determine that a third signal that represents a third intent to move the second portion of the body was generated before a fourth signal that represents a fourth intent to move the first portion of the body. In response, a right child interface element connected to the previous interface element can be accessed. If it is determined that the right child interface element includes two of its own child interface elements, the traversal of the intent-communication interface continues. As a result, the traversal of the intent-communication interface can be performed across subsequent time points, until a particular interface element is reached. From the particular interface element, an interface-operation data associated with the interface element can be accessed based on detecting another biological-signal data that represents an intent to simultaneously move both of the left and right portions of the body. A particular operation to be performed by the computing device (e.g., inputting a “1” numerical character) can then be identified from the interface-operation data. After the operation is performed, the traversal process of the intent-communication interface can be repeated from the root interface element until a targeted outcome (e.g., inputting a complete sentence) is reached.

The intent-communication interface for translating activation sequence of biological signals can be applied or otherwise can enhance various operations associated with the computing device. In some instances, the interface elements of the intent-communication interface identify one or more words or phrases predicted by a machine-learning model. For example, text data previously inputted on the graphical user interface include “the teacher typed into his computer . . . ”. Based on the previous text data, one or more interface elements of the intent-communication interface can be updated to include predicted words or phrases that logically follow the existing text. Continuing with this example, an interface element can include one of the predicted words or phrases such as “keyboard”, “screen”, or “device”, in which the words and phrases are predicted by processing the previous text data using the machine-learning model (e.g., a long short-term memory neural network). In some instances, other interface elements of the intent-communication interface include a set of default alphanumerical characters, to allow the user to input text that are different from the predicted words or phrases. The word prediction based on machine learning can further increase efficiency of performing complex tasks on the graphical user interface.

In some instances, the interface elements of the intent-communication interface identify operations associated with specific types of computing devices, including augmented or virtual reality devices. For example, augmented reality (AR) glasses can display a set of virtual screens. The intent-communication interface can be traversed using biological signals across different time points to select a first virtual screen of the set of virtual screens. Once the first virtual screen is selected, the interface elements of the intent-communication interface can be automatically updated to identify a set of operations (e.g., delete, create a new virtual screen, move to a different location, increase or decrease screen size, modify orientation of the screen), at which the intent-communication interface can be traversed again to identify a particular operation (e.g., increase screen size) from the set of operations. The intent-communication interface can again be automatically updated such that the interface elements identify a subset of operations relating the increasing the screen size (e.g., 1x, 2x, 3x). As a result, multiple traversals of the intent-communication interface can be performed to efficiently perform tasks that are specifically associated with the AR glasses. The techniques for using activation sequence of biological signals can be extended to other types of devices, such as computing devices with robotic components (e.g., a drone device).

Accordingly, certain embodiments described herein improve existing BCIs by implementing techniques that can efficiently translate biological signals of the subject to perform complex tasks. For example, activation sequence of the biological signals can be used to determine various types of operations to be performed by the computing device. Rather than relying on directly translating biological signals to a particular operation, the use of activation sequence and corresponding intent-communication interfaces can reduce potential errors and lead to an efficient performance of computer operations. Moreover, the intent-communication interfaces can be configured to perform different operations across various computing platforms (e.g., robotics, augmented reality devices). Finally, the use of activation sequence of biological signals can be further enhanced using machine-learning techniques to increase efficiency and effectiveness of performing the computing-device operations. Accordingly, embodiments herein reflect an improvement in functions of neural-interface systems and graphical user-interface technology.

### I. System for Analyzing Biological Signals

### A. Multi-Electrode Device

FIG. 1 shows a user 105 using a multi-electrode device 110. The device is shown as being adhered to the user's forehead 115 (e.g., via an adhesive positioned between the device and the user). The device can include multiple electrodes to detect and record neural signals. Subsequent to the signal recording, the device can transmit (e.g., wirelessly transmit) the data (or a processed version thereof) to another electronic device 120, such as a smart phone. The other electronic device 120 can then further process and/or respond to the data, as further described herein. Thus, FIG. 1 exemplifies that multi-electrode device 105 can be small and simple to position. While only one device is shown in this example, it will be appreciated that—in some embodiments—multiple devices are used.

Further, while FIG. 1 illustrates that an adhesive attaches device 110 to user 105, other attachment means can be used. For example, a head harness or band can be positioned around a user and the device. Also, while housing all electrodes for a channel in a single compact unit is often advantageous for ease of use, it will be appreciated that, in other instances, electrodes can be external to a primary device housing and can be positioned far from each other. In one instance, a device as descried in PCT application PCT/US2010/054346 is used. PCT/US2010/054346 is hereby incorporated by reference in its entirety for all purposes.

Devices 115a and 115b can communicate directly (e.g., over a Bluetooth connection or BTLE connection) or indirectly. For example, each device can communicate (e.g., over a Bluetooth connection or BTLE connection) with a server 120, which can be located near tennis court 110.

The biological-signal data collected by the multi-electrode device 110 can include different types of biological signals. For example, the biological-signal data can include EEG data collected from electrodes placed on the subject's forehead. In another example, the biological-signal data can include EMG data collected electrodes of the multi-electrode device 110 that are placed on the subject's limbs. In some instances, the biological-signal data include the following data: (i) an indication of an intent to move a corresponding portion of a body; and (ii) a time point at which the biological signals were generated.

The biological signals collected by the multi-electrode device 110 can be analyzed to detect a signal-activation sequence. For example, detecting the signal-activation sequence can include processing the biological-signal data to identify a first signal and a second signal. The first signal represents an intent to move a first portion of the body of the subject. In some instances, the first signal was generated before the second signal. The second signal can represent another intent to move a second portion of the body of the subject. Thus, detecting the signal-activation sequence can include a determination that the first signals representing the intent to move the first portion of the body of the subject were generated before the second signals representing the other intent to move the second portion of the body of the subject.

The multi-electrode device 1110 can communicate, via short-range connection, the signal-activation sequence of the biological signals to the electronic device 120. The electronic device 120 can process the signal-activation sequence to identify a particular operation. The operation may include inputting one or more alphanumerical characters on a graphical user interface of the computing device. In another example, the operation can include moving a cursor displayed by the graphical user interface. The operations can also include operations that are performed by different types of computing devices, including controlling one or more robot components or controlling augmented reality or virtual reality devices. The electronic device 120 can then perform the identified operation. As such, based on the activation sequence of biological signals, various types of operations can be performed by the electronic device 120. Various embodiments for processing biological-signal data collected from the multi-electrode device 110 are also described in Sections III-VI of the present disclosure.

### B. Example Computing Environment

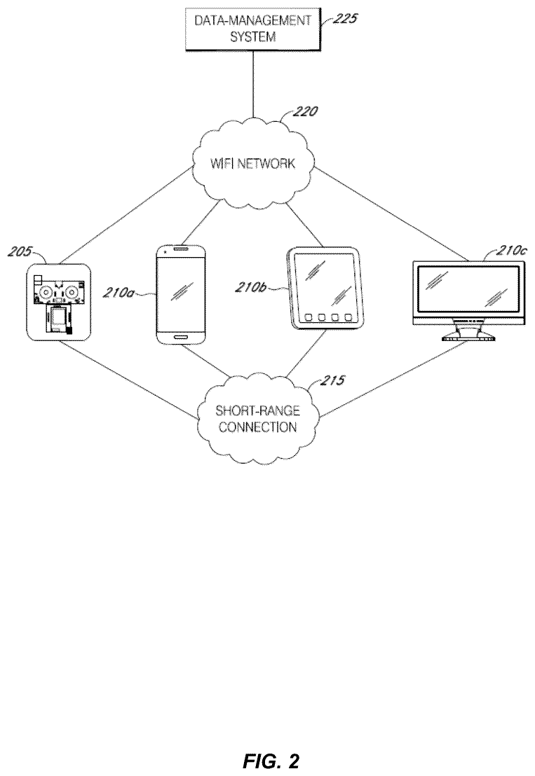

FIG. 2 shows examples of devices connected on a network to facilitate coordinated assessment and use of biological electrical recordings. One or more multi-electrode devices 205 can collect channel data derived from recorded biological data from a user. The biological-signal data can then be presented and processed by one or more other electronic devices, such as a mobile device 210a (e.g., a smart phone), a tablet 210b or laptop or a desktop computer 201c. The one or more devices 201, 205, and/or 210 can analyze the biological-signal data to determine a signal-activation sequence. For example, detecting the signal-activation sequence can include processing the biological-signal data to identify a first signal and a second signal. The first signal represents an intent to move a first portion of the body of the subject. In some instances, the first signal was generated before the second signal. The second signal can represent another intent to move a second portion of the body of the subject. The one or more devices 201, 205, and/or 210 can identify a particular operation that can be performed based on the signal-activation sequence. For example, the particular operation may include inputting one or more alphanumerical characters on a graphical user interface of the computing device.

The inter-device communication can be over a connection, such as a short-range connection 215 (e.g., a Bluetooth, BTLE or ultra-wideband connection) or over a WiFi network 220, such as the Internet.

One or more devices 205 and/or 210 can further access a data-management system 225, which can (for example) receive and assess data from a collection of multi-electrode devices. For example, a health-care provider or pharmaceutical company (e.g., conducting a clinical trial) can use data from multi-electrode devices to measure health of patients. Thus, e.g., data-management system 225 can store data in association with particular users and/or can generate population statistics.

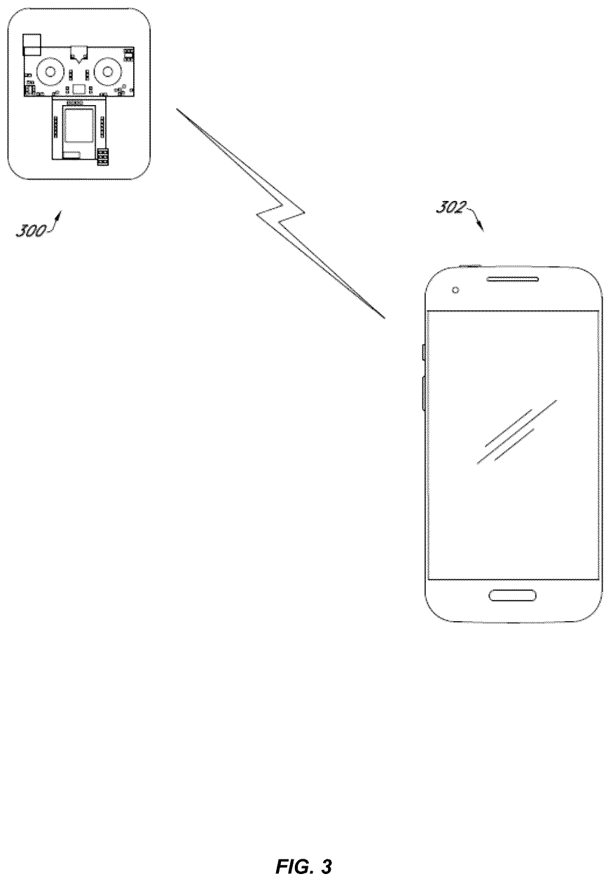

FIG. 3 shows a multi-electrode device 300 communicating (e.g., wirelessly or via a cable) with another electronic device 302. This communication can be performed to enhance a functionality of a multi-electrode device by drawing on resources of the other electronic device (e.g., faster processing speed, larger memory, display screen, input-receiving capabilities). In one instance, electronic device 302 includes interface capabilities that allow for a user (e.g., who may, or may not be, the same person from whom signals are being recorded) to view information (e.g., summaries of recorded data and/or operation options) and/or control operations (e.g., controlling a function of multi-electrode device 300 or controlling another operation, such as speech construction). The communication between devices 300 and 02 can occur intermittently as device 300 collects and/or processes data or subsequent to a data-collection period. The data can be pushed from device 300 to other device 302 and/or pulled from other device 302. For example, the multi-electrode device 300 can push the biological-signal data to the electronic device 302 via a wireless communication network (e.g., a short-range communication network). The electronic device 302 can process the biological-signal data to determine the signal-activation sequence (e.g., determine whether the signals indicate an intent to squeeze a left hand), which can be used to identify a particular operation to be performed by the electronic device 302 and/or another computing device. Various embodiments for processing biological-signal data collected from the multi-electrode device 205 or 300 are also described in Sections III-VI of the present disclosure.

### C. System Architecture

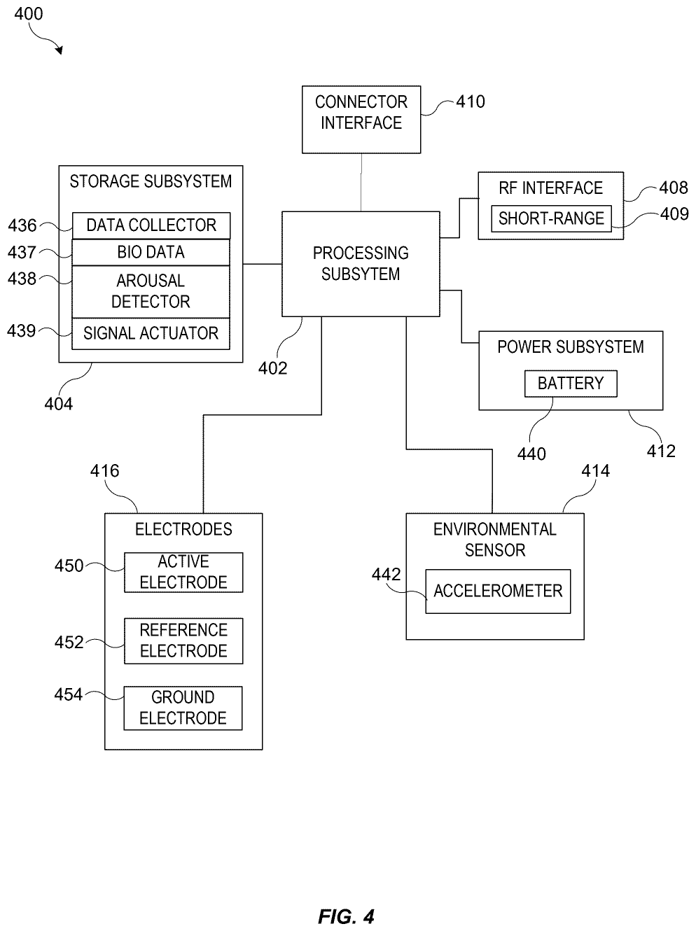

FIG. 4 is a simplified block diagram of a multi-electrode device 400 (e.g., implementing multi-electrode device 300) according to one embodiment. The multi-electrode device 400 can include processing subsystem 402, storage subsystem 404, RF interface 408, connector interface 410, power subsystem 412, environmental sensors 414, and electrodes 416. Multi-electrode device 400 need not include each shown component and/or can also include other components (not explicitly shown).

Storage subsystem 404 can be implemented, e.g., using magnetic storage media, flash memory, other semiconductor memory (e.g., DRAM, SRAM), or any other non-transitory storage medium, or a combination of media, and can include volatile and/or non-volatile media. In some embodiments, storage subsystem 404 can store biological data (e.g., biological-signal data), information (e.g., identifying information and/or medical-history information) about a user and/or analysis variables (e.g., previously determined strong frequencies or frequencies for differentiating between signal groups). In some embodiments, storage subsystem 404 can also store one or more application programs (or apps) 434 to be executed by processing subsystem 410 (e.g., to initiate and/or control data collection, data analysis and/or transmissions).

Processing subsystem 402 can be implemented as one or more integrated circuits, e.g., one or more single-core or multi-core microprocessors or microcontrollers, examples of which are known in the art. In operation, processing system 402 can control the operation of multi-electrode device 400. In various embodiments, processing subsystem 404 can execute a variety of programs in response to program code and can maintain multiple concurrently executing programs or processes. At any given time, some or all of the program code to be executed can be resident in processing subsystem 404 and/or in storage media such as storage subsystem 404.

Through suitable programming, processing subsystem 402 can provide various functionality for multi-electrode device 400. For example, in some embodiments, processing subsystem 402 can execute code that can control the collection, analysis, application and/or transmission of biological data. In some embodiments, some or all of this code can interact with an interface device (e.g., other device 302 in FIG. 3), e.g., by generating messages to be sent to the interface device and/or by receiving and interpreting messages from the interface device. For example, the processing of the biological-signal data can include the processing subsystem 402 providing the biological-signal data to the interface device, at which the interface device (e.g., other device 302 in FIG. 3) can translate the biological-signal data to identify the operations associated with the computing device. In some embodiments, some or all of the code can operate locally to multi-electrode device 400. For example, the storage subsystem 404 can store a signal-processing application for translating the biological-signal data. The processing subsystem 402 of the multi-electrode device 400 can execute the signal-processing application to identify various operations associated with a computing device, the details of which are further described in Sections III-VI of the present disclosure.

Processing subsystem 402 can also execute a data collection code 436, which can cause data detected by electrodes 416 to be recorded and saved. In some instances, signals are differentially amplified and filtering can be applied. The signals can be stored in a biological-data data store 437, along with recording details (e.g., a recording time and/or a user identifier). The data can be further analyzed to detect physiological correspondences. As one example, processing of a spectrogram of the recorded signals can reveal frequency properties that correspond to particular sleep stages. As another example, an arousal detection code 438 can analyze a gradient of the spectrogram to identify and assess sleep-disturbance indicators and detect arousals. As yet another example, a signal actuator code 439 can translate particular biological-signal features into a motion of an external object (e.g., a cursor). For example, the signal actuator code 439 can be used to identify biological-signal data that correspond to an intent to move a particular portion of a body (e.g., left hand) of a subject, which can then be translated to a particular operation to be performed by a computing device. Such techniques and codes are further described herein.

RF (radio frequency) interface 408 can allow multi-electrode device 400 to communicate wirelessly with various interface devices. RF interface 408 can include RF transceiver components such as an antenna and supporting circuitry to enable data communication over a wireless medium, e.g., using Wi-Fi (IEEE 802.11 family standards), Bluetooth® (a family of standards promulgated by Bluetooth SIG, Inc.), or other protocols for wireless data communication. In some embodiments, RF interface 408 can implement a short-range sensor (e.g., Bluetooth, BLTE or ultra-wide band) proximity sensor 409 that supports proximity detection through an estimation of signal strength and/or other protocols for determining proximity to another electronic device. In some embodiments, RF interface 408 can provide near-field communication (“NFC”) capability, e.g., implementing the ISO/IEC 18092 standards or the like; NFC can support wireless data exchange between devices over a very short range (e.g., 20 centimeters or less). RF interface 408 can be implemented using a combination of hardware (e.g., driver circuits, antennas, modulators/demodulators, encoders/decoders, and other analog and/or digital signal processing circuits) and software components. Multiple different wireless communication protocols and associated hardware can be incorporated into RF interface 408.

Connector interface 410 can allow multi-electrode device 400 to communicate with various interface devices via a wired communication path, e.g., using Universal Serial Bus (USB), universal asynchronous receiver/transmitter (UART), or other protocols for wired data communication. In some embodiments, connector interface 410 can provide a power port, allowing multi-electrode device 400 to receive power, e.g., to charge an internal battery. For example, connector interface 410 can include a connector such as a mini-USB connector or a custom connector, as well as supporting circuitry. In some embodiments, the connector can be a custom connector that provides dedicated power and ground contacts, as well as digital data contacts that can be used to implement different communication technologies in parallel; for instance, two pins can be assigned as USB data pins (D+ and D−) and two other pins can be assigned as serial transmit/receive pins (e.g., implementing a UART interface). The assignment of pins to particular communication technologies can be hardwired or negotiated while the connection is being established. In some embodiments, the connector can also provide connections to transmit and/or receive biological electrical signals, which can be transmitted to or from another device (e.g., device 302 or another multi-electrode device) in analog and/or digital formats.

Environmental sensors 414 can include various electronic, mechanical, electromechanical, optical, or other devices that provide information related to external conditions around multi-electrode device 400. Sensors 414 in some embodiments can provide digital signals to processing subsystem 402, e.g., on a streaming basis or in response to polling by processing subsystem 402 as desired. Any type and combination of environmental sensors can be used; shown by way of example is an accelerometer 442. Acceleration sensed by accelerometer 442 can be used to estimate whether a user is or is trying to sleep and/or estimate an activity state.

Electrodes 416 can include, e.g., round surface electrodes and can include gold, tin, silver, and/or silver/silver-chloride. Electrodes 416 can have a diameter greater than ⅛″ and less than 1″. Electrodes 416 can include an active electrode 450, a reference electrode 452 and (optionally) ground electrode 454. The electrodes may or may not be distinguishable from each other. The electrodes location can be fixed within a device and/or movable (e.g., tethered to a device). In some embodiments, some of the electrodes 416 are configured to collect EEG data. Additionally or alternatively, other electrodes can be configured to collect EMG data.

Power subsystem 412 can provide power and power management capabilities for multi-electrode device 400. For example, power subsystem 414 can include a battery 440 (e.g., a rechargeable battery) and associated circuitry to distribute power from battery 440 to other components of multi-electrode device 400 that require electrical power. In some embodiments, power subsystem 412 can also include circuitry operable to charge battery 440, e.g., when connector interface 410 is connected to a power source. In some embodiments, power subsystem 412 can include a “wireless” charger, such as an inductive charger, to charge battery 440 without relying on connector interface 410. In some embodiments, power subsystem 412 can also include other power sources, such as a solar cell, in addition to or instead of battery 440.

It will be appreciated that multi-electrode device 400 is illustrative and that variations and modifications are possible. For example, multi-electrode device 400 can include a user interface to enable a user to directly interact with the device. As another example, multi-electrode device can have an attachment indicator that indicates (e.g., via a light color or sound) whether a contact between a device and a user's skin is adequate and/or whether recorded signals are of an acceptable quality.

Further, while the multi-electrode device is described with reference to particular blocks, it is to be understood that these blocks are defined for convenience of description and are not intended to imply a particular physical arrangement of component parts. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments can be realized in a variety of apparatus including electronic devices implemented using any combination of circuitry and software. It is also not required that every block in FIG. 4 be implemented in a given embodiment of a multi-electrode device.

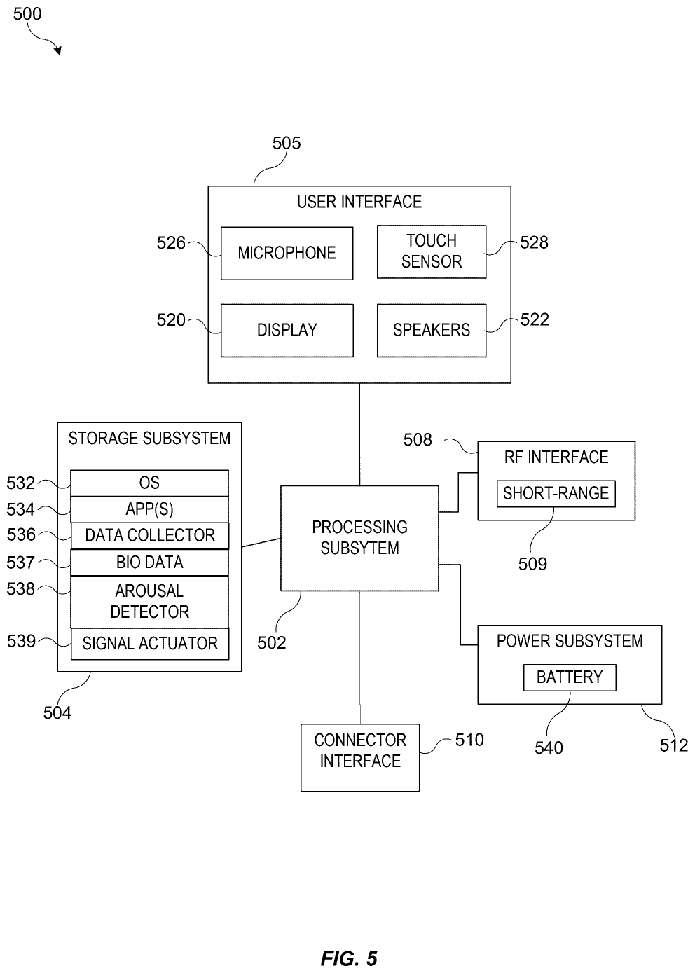

An interface device such as device 302 of FIG. 3 can be implemented as an electronic device using blocks similar to those described above (e.g., processors, storage media, RF interface, etc.) and/or other blocks or components. FIG. 5 is a simplified block diagram of an interface device 500 (e.g., implementing device 302 of FIG. 3) according to one embodiment. Interface device 500 can include processing subsystem 502, storage subsystem 504, user interface 506, RF interface 508, connector interface 510 and power subsystem 512. Interface device 500 can also include other components (not explicitly shown). Many of the components of interface device 500 can be similar or identical to those of multi-electrode device 300 of FIG. 3.

For instance, storage subsystem 504 can be generally similar to storage subsystem 404 and can include, e.g., using magnetic storage media, flash memory, other semiconductor memory (e.g., DRAM, SRAM), or any other non-transitory storage medium, or a combination of media, and can include volatile and/or non-volatile media. Like storage subsystem 504, storage subsystem 504 can be used to store data and/or program code to be executed by processing subsystem 502. For example, the storage subsystem 504 can store a signal-processing application for translating the biological-signal data to identify various operations associated with a computing device.

User interface 506 can include any combination of input and output devices. A user can operate input devices of user interface 506 to invoke the functionality of interface device 500 and can view, hear, and/or otherwise experience output from interface device 500 via output devices of user interface 506. Examples of output devices include display 520 and speakers 522. Examples of input devices include microphone 526 and touch sensor 528.

Display 520 can be implemented using compact display technologies, e.g., LCD (liquid crystal display), LED (light-emitting diode), OLED (organic light-emitting diode), or the like. In some embodiments, display 520 can incorporate a flexible display element or curved-glass display element, allowing interface device 500 to conform to a desired shape. One or more speakers 522 can be provided using small-form5factor speaker technologies, including any technology capable of converting electronic signals into audible sound waves. Speakers 522 can be used to produce tones (e.g., beeping or ringing) and/or speech. In some instances, the display 520 display an intent-communication interface. The biological-signal data can be translated to access interface-operation data from the intent-communication interface, in which the interface-operation data is used by the signal-processing application to identify a particular operation to be performed by the computing device. Various embodiments for implementing the intent-communication interfaces are described in Sections III-VI of the present disclosure.

Examples of input devices include microphone 526 and touch sensor 528. Microphone 526 can include any device that converts sound waves into electronic signals. In some embodiments, microphone 526 can be sufficiently sensitive to provide a representation of specific words spoken by a user; in other embodiments, microphone 426 can be usable to provide indications of general ambient sound levels without necessarily providing a high-quality electronic representation of specific sounds.

Touch sensor 528 can include, e.g., a capacitive sensor array with the ability to localize contacts to a particular point or region on the surface of the sensor and in some instances, the ability to distinguish multiple simultaneous contacts. In some embodiments, touch sensor 428 can be overlaid over display 520 to provide a touchscreen interface, and processing subsystem 504 can translate touch events into specific user inputs depending on what is currently displayed on display 520.

Processing subsystem 502 can be implemented as one or more integrated circuits, e.g., one or more single-core or multi-core microprocessors or microcontrollers, examples of which are known in the art. In operation, processing system 502 can control the operation of interface device 500. In various embodiments, processing subsystem 502 can execute a variety of programs in response to program code and can maintain multiple concurrently executing programs or processes. At any given time, some or all of the program code to be executed can be resident in processing subsystem 502 and/or in storage media such as storage subsystem 504. For example, the processing subsystem 502 can access the biological-signal data provided by a multi-electrode device (e.g., the multi-electrode device 400) and execute the signal-processing application to translate the biological-signal data to identify various operations associated with a computing device. Translating the biological-signal data can include determining a signal-activation sequence, such as processing the biological-signal data to identify a first signal and a second signal. The first signal represents an intent to move a first portion of the body of the subject. In some instances, the first signal was generated before the second signal. The second signal can represent another intent to move a second portion of the body of the subject. The signal-processing application can identify a particular operation that can be performed based on the signal-activation sequence. For example, the particular operation may include inputting one or more alphanumerical characters on a graphical user interface of the computing device.

Through suitable programming, processing subsystem 502 can provide various functionality for interface device 500. For example, in some embodiments, processing subsystem 502 can execute an operating system (OS) 532 and various applications 534. In some embodiments, some or all of these application programs can interact with a multi-electrode device, e.g., by generating messages to be sent to the multi-electrode device and/or by receiving and interpreting messages from the multi-electrode device. In some embodiments, some or all of the application programs can operate locally at interface device 500.

Processing subsystem 502 can also execute a data-collection code 536 (which can be part of OS 532, part of an app or separate as desired). Data-collection code 536 can be, at least in part, complementary to data-collection code 436 in FIG. 4. In some instances, data-collection code 536 is configured such that execution of the code causes device 500 to receive raw or processed biological-signal data (e.g., EEG or EMG signals) from a multi-electrode device (e.g., multi-electrode device 300 of FIG. 3), in which the biological electric signals can indicate an intent to move a particular portion of a body of the subject. Data-collection code 536 can further define processing to perform on the received data (e.g., to apply filters, generate metadata indicative of a source multi-electrode device or receipt time, and/or compress the data). Data-collection code 536 can further, upon execution, cause the raw or processed biological electrical signals to be stored in a biological data store 537.

In some instances, execution of data-collection code 536 further causes device 500 to collect data, which can include other biological data (e.g., a patient's temperature or pulse) or external data (e.g., a light level or geographical location). This information can be stored with the biological-signal data (e.g., such that metadata for an EEG or EMG recording includes a patient's temperature and/or location) and/or can be stored separately (e.g., with a timestamp to enable future time-synched data matching). It will be appreciated that, in these instances, interface device 500 can either include the appropriate sensors to collect this additional data (e.g., a camera, thermometer, GPS receiver) or can be in communication (e.g., via RF interface 508) with another device with such sensors.

Processing subsystem 502 can also execute one or more codes that can, in real-time or retrospectively, analyze raw or processed biological electrical signals (i.e., the biological-signal data) to detect events of interest. For example, execution of an arousal-detection code 538 can assess changes with a spectrogram (built using EEG data) corresponding to a sleep period of a patient to determine whether and/or when arousals occurred. In one instance, this assessment can include determining-for each time increment-a change variable corresponding to an amount by which power (e.g., normalized power) at one or more frequencies for the time increment changed relative to one or more other time increments. In one instance, this assessment can include assigning each time increment to a sleep stage and detecting time intervals at which the assignments changed. Sleep-staging categorizations can (in some instances) further detail any arousals that are occurring (e.g., by indicating in which stages arousals occur and/or by identifying through how many sleep stages an arousal traversed).

As another example, execution of a signal actuator code 539 can assess and translate EEG and/or EMG data that represent an intent to move a portion of the body (e.g., left hand) of the subject to identify various operations associated with the computing device. Initially, a mapping can be constructed to associate particular EEG and/or EMG signatures with particular actions. The actions can be external actions, such as actions of a cursor on a screen. For example, the actions can include controlling a robotic component of another device or inputting data on a graphical user interface. The mapping can be performed using a clustering and/or component analysis and can utilize raw or processed signals recorded from one or more active electrodes (e.g., from one or more multi-electrode devices, each positioned on a different muscle).

In one instance, execution of signal actuator code 539 causes an interactive visualization to be presented on display 520. A cursor position on the screen can be controlled based on a real-time analysis of EEG and/or EMG data using the mapping. A person from whom the recordings are collected from can thus interact with the interface without using his hands. In an exemplary instance, the visualization can include a speech-assistance visualization that allows a person to select letters, series of letters, words or phrases. A sequential selection can allow the person to construct sentences, paragraphs or conversations. The text can be used electronically (e.g., to generate an email or letter) or can be verbalized (e.g., using a speech component of signal actuator 539 to send audio output to speakers 522) to communicate with others nearby.

RF (radio frequency) interface 508 and/or connector interface 510 can allow interface device 500 to communicate wirelessly with various other devices (e.g., multi-electrode device 400 of FIG. 4) and networks. RF interface 508 can correspond to (e.g., include a described characteristic of) RF interface 408 from FIG. 4 and/or connector interface 510 can correspond to (e.g., include a described characteristic of) connector interface 410. Power subsystem 512 can provide power and power management capabilities for interface device 512. Power subsystem 512 can correspond to (e.g., include a described characteristic of) power subsystem 41.

It will be appreciated that interface device 500 is illustrative and that variations and modifications are possible. In various embodiments, other controls or components can be provided in addition to or instead of those described above. Any device capable of interacting with another device (e.g., multi-electrode device) to store, process and/or use recorded biological electrical signals can be an interface device.

Further, while the interface device is described with reference to particular blocks, it is to be understood that these blocks are defined for convenience of description and are not intended to imply a particular physical arrangement of component parts. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments can be realized in a variety of apparatus including electronic devices implemented using any combination of circuitry and software. It is also not required that every block in FIG. 5 be implemented in a given embodiment of a mobile device.

Communication between one or more multi-electrode devices, one or more mobile devices and an interface device can be implemented according to any communication protocol (or combination of protocols) that both devices are programmed or otherwise configured to use. In some instances, standard protocols such as Bluetooth protocols or ultra-wideband protocols can be used. In some instances, a custom message format and syntax (including, e.g., a set of rules for interpreting particular bytes or sequences of bytes in a digital data transmission) can be defined, and messages can be transmitted using standard serial protocols such as a virtual serial port defined in certain Bluetooth standards. Embodiments are not limited to particular protocols, and those skilled in the art with access to the present teachings will recognize that numerous protocols can be used.

In accordance with certain embodiments, one or more multi-electrode devices can be conveniently used to collect electrical biological data from a patient. The data can be processed to identify signals of physiological significance. The detection itself can be useful, as it can inform a user or a third party about a patient's health and/or efficacy of a current treatment. In some instances, the signals can be used to automatically control another object, such as a computer cursor. Such a capability can extend a user's physical capabilities (e.g., which may be handicapped due to a disease) and/or improve ease of operation.

### II. Methods for Analyzing Biological Signals to Identify an Intent to Move a Portion of the Body

To facilitate translation of biological signals (e.g., electroencephalography (EEG) data, electromyography (EMG) data) for identifying various computing operations, machine-learning or statistical-analysis techniques can be used to identify biological-signal data representing an intent to move a particular portion of a body of a subject (e.g., left hand, right hand). For example, one or more signal-processing analyses (e.g., independent-component analysis (ICA)) can be used to identify a reference signature of the biological-signal data that can be used to determine whether biological signals obtained from a different subject correspond to an intent to move the particular portion of the body.

As an illustrative example, reference dataset that includes a set of biological-signal data (e.g., EEG data) representing left-and right-hand movement imaginations can be collected. For example, each of the set the biological-signal data of the reference dataset can include 32-channel EEG signals recorded by a multi-electrode device (e.g., the multi-electrode device 110 of FIG. 1), in which the biological-signal data can be recorded for a corresponding subject who performs an intended movement of a left hand or right hand (e.g., move a cursor to a left interface element of an intent-communication tree, squeeze the left hand). In some instances, a biological-signal data of the set the biological-signal data also includes a base, non-movement state of the corresponding subject. Each biological-signal data of the reference dataset can then be decomposed into one or more independent components (ICs) that represent the biological-signal data.

In some instances, the set the biological-signal data are first projected to a 15-dimensional subspace using principal component analysis (PCA), at which the PCA components can be further processed to generate the ICs. PCA can be used to reduce the dimensionality of the biological-signal data of the reference dataset. Implementing the ICA of biological signals with PCA can be advantageous, because PCA can significantly reduce the computation time and the need of large amounts of computer memory.

One or more biological-signal signatures that represent an intent to move the particular portion of the body of the subject can then be identified from the ICs of the reference dataset. In some instances, the one or more biological-signal signatures are selected from the ICs that best represent the intent to move the particular portion of the body. For example, the biological-signal signatures are identified based at least in part on the spatial pattern of the ICs that correlate with activation of the sensorimotor cortex of a corresponding brain hemisphere. The biological-signal signatures can be used as a reference signature for classifying whether biological signals collected from another subject represent an intent to move a left or right portion of the body. In addition to using ICA, other types of signal analyses can be used to identify biological signals that represent an intended movement of a portion of the body, as contemplated by one skilled in the art.

### III. Methods for Analyzing Spectrogram Data to Identify an Intent to Move a Portion of the Body

### A. Collecting Biological Signal Data

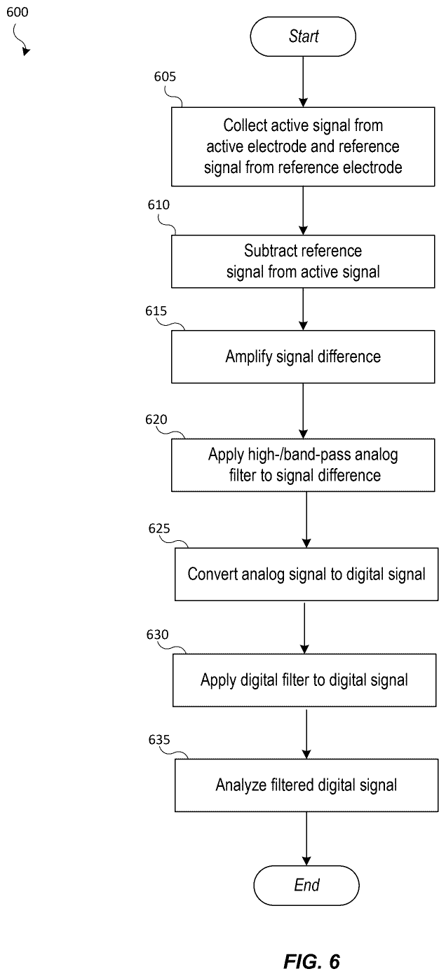

FIG. 6 is a flow diagram of a process 600 for using a multi-electrode device to collect a channel of biological electrode data according to an embodiment. Part of all of process 600 can be implemented in a multi-electrode device (e.g., multi-electrode device 400). In some instances, part of process 600 (e.g., one or more of blocks 610-635) can be implemented in an electronic device that is remote from a multi-electrode device, where the blocks can be performed immediately after receiving signals from a multi-electrode device (e.g., immediately after collection), prior to storing data pertaining to a recording, in response to a request relying on collected data and/or prior to using the collected data.

At block 605, an active signal and a reference signal can be collected using respective electrodes. In some instances, a ground signal is further collected from a ground electrode. The active electrode and the reference electrode and/or the active electrode and the ground electrode can be attached to a single device (e.g., a multi-electrode device), a fixed distance from each other and/or are close to each other (e.g., such that centers of the electrodes are located less than 12, 6 or 4 inches from each other and/or such that the electrodes are positioned to likely record signals from a same muscle or same brain region).

In some instances, the reference electrode is positioned near the active electrode, such that both electrodes will likely sense electrical activity from a same brain region or from a same muscle. For example, a first active electrode positioned near a first reference electrode can be used to collect first biological signals (e.g., EEG) generated from a left hemisphere region of the brain of a subject, in which the first biological signals represent an intent to move a right limb of the body of the subject. Similarly, a second active electrode positioned near a second reference electrode can be used to collect second biological signals generated from a right hemisphere region of the brain of the subject, in which the second biological signals represent an intent to move a left limb of the body of the subject. The sequence of when the first and second biological signals were detected can be used to identify a particular operation associated with computing devices. In other instances, the reference electrode is positioned further from the active electrode (e.g., at an area that is relatively electrically neutral, which may include an area not over the brain or a prominent muscle) to reduce overlap of a signal of interest.

Prior to the collection, the electrodes can be attached to a skin of a person. This can include, e.g., attaching a single device completely housing one or more electrodes and/or attaching one or more individual electrodes (e.g., flexibly extending beyond between a device housing). In one instance, such attachment is performed by using an adhesive (e.g., applying an adhesive substance to at least part of an underside of a device, applying an adhesive patch over and around the device and/or applying a double-sided adhesive patch under at least part of the device) to attach a multi-electrode device including the active and reference electrodes to a person. For an EEG recording, the device can be attached, e.g., near the person's frontal lobe (e.g., on her forehead). For an EMG recording, the device can be attached over a muscle (e.g., over a jaw muscle or neck muscle).

In some instances, only one active signal is recorded at a time. In other instances, each of a set of active electrodes records an active signal. In this situation, the active electrodes can be positioned at different body locations (e.g., on different sides of the body, on different muscle types or on different brain regions). For example, for the EMG recording, the active electrodes of the device are attached over left and right limbs of the body of the subject, such that signal-activation sequence can be determined to identify various operations associated with the computing device. Each active electrode can be associated with a reference electrode or fewer references may be collected relatively to a collected number of active signals. Each active electrode can be present in a separate multi-electrode device.

At block 610, the reference signal can be subtracted from the active electrode. This can reduce noise in the active signal, such as recording noise or noise due to a patient's breathing or movement. Though proximate location of the reference and active electrodes has been traditionally shunned, such locations can improve the portion of the active electrode's noise (e.g., patient movement noise) that will be shared at the reference electrode noise. For example, if a patient is rolling over, a movement that will be experienced by an active electrode positioned over brain centre F7 will be quite different from movement experienced by a reference electrode positioned on a contralateral ear. Meanwhile, if both electrodes are positioned over a same F7 region, they will likely experience similar movement artifacts. While the signal difference may lose representation of some cellular electrical activity from an underlying physiological structure, a larger portion of the remaining signal can be attributed to such activity of interest (due to the removal of noise).

At block 615, the signal difference can be amplified. An amplification gain be, e.g., between 100 and 100,000. At block 620, the amplified signal difference can be filtered. The applied filter can include, e.g., an analog high-pass or band-pass filter. The filtering can reduce signal contributions from flowing potentials, such as breathing. The filter can include a lower cut-off frequency around 0.1-1 Hz. In some instances, the filter can also include a high cut-off frequency, which can be set to a frequency less than a Nyquist frequency determined given based on a sampling rate.

The filtered analog signal can be converted to a digital signal at block 625. A digital filter can be applied to the digital signal at block 630. Digital filter can reduce DC signal components. Digital filtering can be performed using a linear or non-linear filter. Filters can include, e.g., a finite or infinite impulse response filter or a window function (e.g., a Hanning, Hamming, Blackman or rectangular function). Filter characteristics can be defined to reduce DC signal contributions while preserving high-frequency signal components.

The filtered signal can be analyzed at block 635. As described in further detail herein, the analysis can include micro-analyses, such as categorizing individual segments of the signal (e.g., into sleep stages, arousal or non-arousal and/or intent to move). The analysis can alternatively or additionally include macro-analyses, such as characterizing an overall sleep quality or muscle activity.

As noted above, in some instances, multiple devices cooperate to perform process 600. For example, a multi-electrode device 400 of FIG. 4 can perform blocks 605-625, and a remote device (e.g., a server, computer, smart phone or interface device 405) can perform blocks 630-635. It will be appreciated that to facilitate such shared process operation, devices can communicate to share appropriate information. For example, after block 625, a multi-electrode device 400 can transmit the digital signal (e.g., using a short-range network or WiFi network) to another electronic device, such as interface device 500 of FIG. 5. The other electronic device can receive the signal and then perform blocks 630-635.

Though not explicitly shown in process 600, raw and/or processed data can be stored. The data can be stored on a multi-electrode device, a remote device and/or in the cloud. In some instances, both the raw data and a processed version thereof (e.g., identifying classifications associated with portions of the data) can be stored.

It will further be appreciated that process 600 can be an ongoing process. For example, active and reference signals can be continuously or periodically collected over an extended time period, until all operations are performed to reach a target outcome (e.g., inputting text in a graphical user interface). Part or all of process 600 can be performed in real-time as signals are collected and/or data can be fully or partly processed in batches. For example, during a recording session, blocks 605-635 can be performed in real-time at each time point of a set of time points, to facilitate input of each character of text into the graphical user interface.

B. Identifying Frequency Signatures from Biological Signal Data

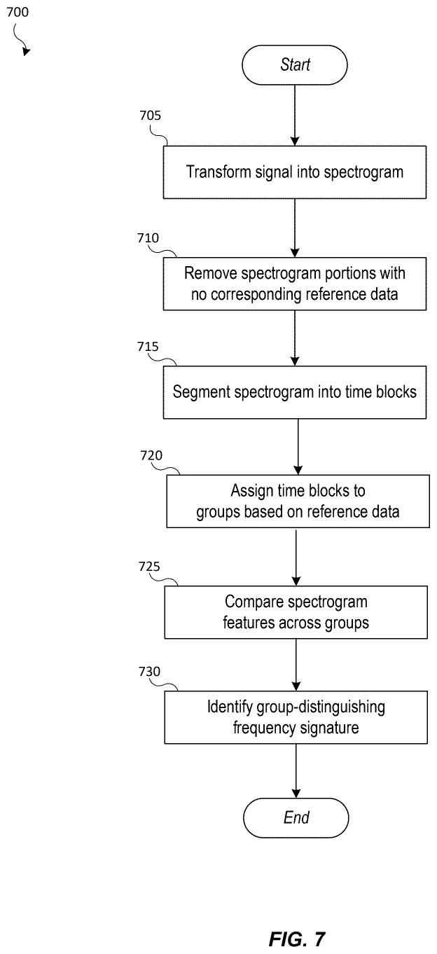

FIG. 7 is a flow diagram of a process 700 for analyzing channel biological data to identify frequency signatures of various biological stages according to an embodiment. Part of all of process 700 can be implemented in a multi-electrode device (e.g., multi-electrode device 400 of FIG. 4) and/or in an electronic device remote from a multi-electrode device (e.g., interface device 500 of FIG. 5).

At block 705, a signal can be transformed into a spectrogram. The signal can include a signal based on recordings from electrodes positioned on a person, such as a differentially amplified and filtered signal. The spectrogram can be generated by parsing a signal into time bins, and computing-for each time bin-a spectrum (e.g., using a Fourier transformation). Thus, the spectrogram can include a multi-dimensional power matrix, with the dimensions corresponding to time and frequency.

Select portions of the spectrogram can, optionally, be removed at block 710. These portions can include those associated with particular time bins, for which it can be determined that a signal quality is poor and/or for which there is no or inadequate reference data. For example, to develop a translation or mapping from signals to physiological events (e.g., an intent to move a particular portion of a body), signatures of various physiological events can be determined using reference data (e.g., corresponding to a human evaluation of the data). Data portions for which no reference data is available can thus be ignored while determining the signatures.

At block 715, the spectrogram can be segmented into a set of time blocks or epochs. Each time block can be of a same duration (e.g., 30 seconds) and can (in some instances) include multiple (e.g., and a fixed number) of time increments, where time increments correspond to each recording time. In some instances, a time block is defined as a single time increment in the spectrogram. In some instances, a time block is defined as multiple time increments. A duration of the time blocks can be determined based on, e.g., a timescale of a physiological event of interest (e.g., 2-second time block to identify signals representing the intent to move the portion of the body); a temporal precision or duration of corresponding reference data; and/or a desired precision, accuracy and/or speed of signal classification.

Each time bin in each time block can be assigned to a group based on reference data at block 720. For example, human scoring of EEG data can identify an intent to move a corresponding portion of the body (e.g., intent to squeeze left hand) for each time block. Time bins in a given time block can then be associated with the corresponding portion of the body. Time bins in a time block can then be assigned to a “left portion” group (if an intent to move the left portion of the body has occurred during the block) or a “right portion” group (if an intent to move the right portion of the body). Similarly, for a given EMG recording, a patient can indicate an intent to move a particular portion of the body. To illustrate, after moving a finger at the right hand, the patient can indicate that he intended for a cursor associated with an intent-communication interface to move from a root interface element to a right child interface element. Time bins associated with the jaw contraction can then be assigned to a “right portion” group.

At block 725, spectrogram features can be compared across groups. In one instance, one or more spectrum features can first be determined for each time bin, and these set of features can be compared at block 725. For example, a strong frequency or fragmentation value can be determined, as described in greater detail herein. As another example, power (or normalized power) at each of one or more frequencies for individual time bins can be compared. In another instance, a collective spectrum can be determined based on spectrums associated with time bins assigned to a given group, and a feature can then be determined based on the collective spectrum. For example, a collective spectrum can include an average or median spectrum, and a feature can include a strong frequency, fragmentation value, or power (at one or more frequencies). As another example, a collective spectrum can include—for each time bin—a feature can include an n1% power (a power where n1% of powers at that frequency are below that power) and an n2% power (a power where n2% of powers at that frequency are below that power).

Using the features, one or more group-distinguishing frequency signatures can be identified at block 730. A frequency signature can include an identification of a variable to identify or determine based on a given spectrum to use for a group assignment. The variable can then be used as part of the reference data (for example) to improve detection of biological signals that represent an intent to move a particular portion of the body. For example, a group-distinguishing frequency signature can include a particular frequency, such that a power at that frequency is to be used for group assignment. As another example, a group-distinguishing frequency can include a weight associated with each of one or more frequencies, such that a weighted sum of the frequencies' powers is to be used for group assignment.

A frequency signature can include a subset of frequencies and/or a weight for one or more frequencies. For example, an overlap between power distributions for two or more groups can be determined, and a group-distinguishing frequency can be identified as a frequency with a below-threshold overlap or as frequency with a relatively small (or a smallest) overlap. In one instance, a model can be used to determine which frequencies' (or frequency's) features can be reliably used to distinguish between the groups. In one instance, a group-distinguishing signature can be identified as a frequency associated with an information value (e.g., based on an entropy differential) above an absolute or relative (e.g., relative to other frequencies' values) values.

In one instance, block 730 can include assigning a weight to each of two or more frequencies. Then, in order to subsequently determine which group a spectrum is to be assigned to, a variable can be calculated that is a weighted sum of (normalized or unnormalized) powers. For example, block 725 can include using a component analysis (e.g., principal component analysis or independent component analysis), and block 730 can include identifying one or more components.

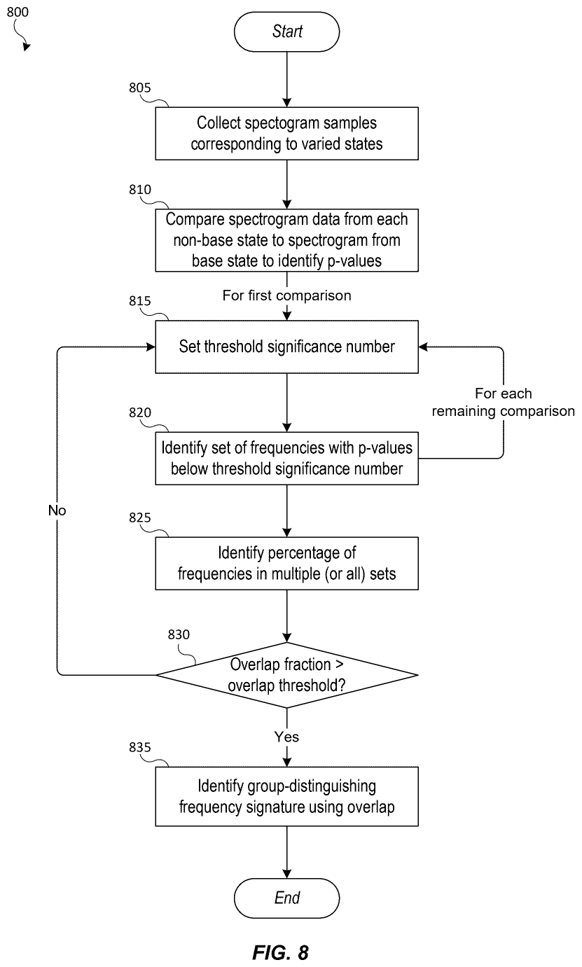

FIG. 8 is a flow diagram of a process 800 for analyzing channel biological data to identify frequency signatures of intended movements according to an embodiment. Part of all of process 800 can be implemented in a multi-electrode device (e.g., multi-electrode device 400 of FIG. 4) and/or in an electronic device remote from a multi-electrode device (e.g., interface device 500 of FIG. 5).

At block 805, spectrogram samples corresponding to various physiological states can be collected. In some instances, at least some states correspond to an intent to move corresponding portions of the body with particular attributes. For example, samples can be collected both from a period in which left arm muscles have been activated and another period in which right arm muscles have been activated, such that the samples an include data that represent an intent to move corresponding muscles of the body. In some instances, the collected samples are based on recordings from a single individual. In another, they are based on recordings from multiple individuals.

In some instances, at least some states correspond to intention states. For example, samples (e.g., based on EMG data) can be collected such that some data corresponds to an intention to induce a particular action (e.g., squeeze a right hand) and other data corresponds to no such.

The spectrogram data can include a spectrogram of raw data, a spectrogram of filtered data, a once-normalized spectrogram (e.g., normalizing a power at each frequency based on powers across time bins for the same frequency or based on powers across frequencies for the same time bin), or a spectrogram normalized multiple times (e.g., normalizing a power at each frequency at least once based on normalized or unnormalized powers across time bins for the same frequency and at least once based on normalized or unnormalized powers across frequencies for the same time bin).

At block 810, spectrogram data from a base state (e.g., a non-action stage) can be compared to spectrogram data from each of one or more non-bases state (e.g., intent to move a particular portion of the body, action state) to identify a significance value. In one instance, for a comparison between the base state and a single non-base state, a frequency-specific significance value can include a p-value and can be determined for each frequency based on a statistical test of the distributions of powers in the two states.

Blocks 815-820 are then performed for each pairwise comparison between a non-base state (e.g., action state) and a base state (e.g., non-action state). A threshold significance number can be set at block 815. The threshold can be determined based on a distribution of the set of frequency-specific significance values and a defined percentage (n %). For example, the threshold significance number can be defined as a value at which n % (e.g., 60%) of the frequency-specific significance values are below the threshold significance number.

A set of frequencies with frequency-specific significance values below the threshold can be identified at block 820. Thus, these frequencies can include those that (based on the threshold significance number) sufficiently distinguish the base state from the non-base state.

Blocks 815 and 820 are then repeated for each additional comparison between the base state and another non-base state. A result then includes a set of an n %-most significant frequencies associated with each non-base state.

At block 825, frequencies present in all sets (or a threshold number of sets) are identified. Thus, the identified overlapping frequencies can include those amongst the n %-most significant frequencies in distinguishing each of multiple non-base states from a base state.

A determination can be made, at block 830, as to whether the overlap percentage is greater than an overlap threshold. When it is not, process 800 can return to block 815, where a new (e.g., higher) threshold significance number can be set. For example, a threshold percentage (n %) used to define the threshold significance number can be incremented (e.g., by 1%), so as to include more frequencies in the set identified at block 820.

When the overlap is determined to be greater than the overlap threshold, process 800 can continue to block 835, where one or more group-distinguishing frequency signatures can be defined using frequencies in an overlap between the sets. The signature can include an identification of a subset of frequencies in the spectrogram and/or a weight for each of one or more frequencies. The weight can be based on, e.g., a frequency's frequency-specific significance values for each of one or more base-state versus non-base-state comparisons or (in instances where the overlap assessment does not require that the identified frequencies be present in all sets of frequencies) a number of sets that include a given frequency. In some instances, the signature includes one or more components defined by assigning weights frequencies in the overlap. For example, a component analysis can be performed using state assignments and powers at frequencies in the overlap to identify one or more components.

Subsequent analyses (e.g., of different data) can be focused on the group-distinguishing frequency signature(s). In some instances, a spectrogram (e.g., normalized or unnormalized spectrogram) can be cropped to exclude frequencies not defined as being a group-defining frequency. For example, process 800 can be initially performed to identify group-defining frequencies, and process 700 (e.g., subsequently analyzing different data) can crop a signal's spectrogram using the group-defining frequencies before comparing.

### C. Normalizing Spectrogram Data

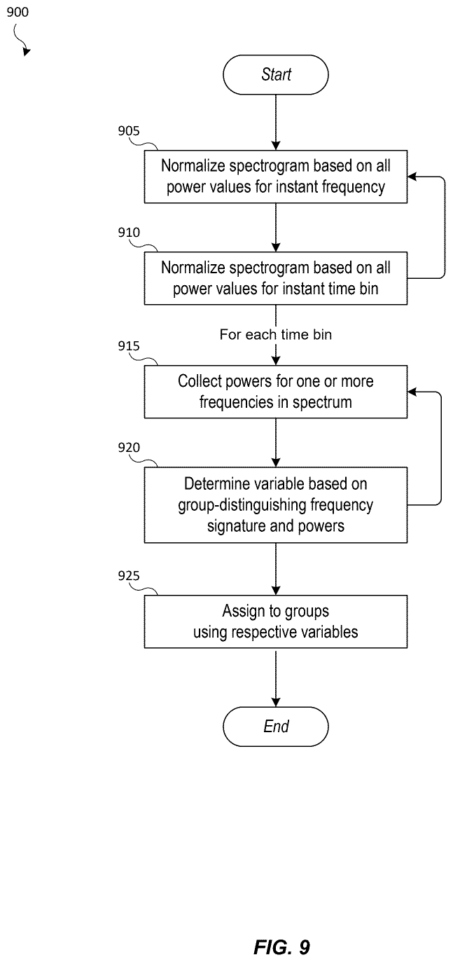

FIG. 9 is a flow diagram of a process 900 for normalizing a spectrogram and using a group-distinguishing frequency signature to classify biological data according to an embodiment. Part of all of process 900 can be implemented in a multi-electrode device (e.g., multi-electrode device 400 of FIG. 4) and/or in an electronic device remote from a multi-electrode device (e.g., interface device 500 of FIG. 5).

At blocks 905 and 910, a spectrogram built from recorded biological electrical signals (e.g., EEG or EMG data) is normalized (e.g., once, multiple times or iteratively). In some embodiments, the spectrogram is built from channel data for one or more channels, each generated based on signals recorded using a device that fixes multiple electrodes relative to each other or that tethers multiple electrodes to each other.

A first normalization, performed at block 905, can be performed by first determining—for each frequency in the spectrogram—a z-score of the powers associated with that frequency (i.e., across all time bins). The powers at that frequency can then be normalized using this z-score value.

A (optional) second normalization, performed at block 910, can be performed by first determining—for each time bin in the spectrogram—a z-score based on the powers associated with that time bin (i.e., across all time bins). The powers at that time bin can then be normalized using this z-score value.

These normalizations can be repeatedly performed (in an alternating manner) a set number of times or until a normalization factor (or a change in a normalization factor) is below a threshold. In some instances, only one normalization is performed, such that either block 905 or block 910 is omitted from process 900. In some instances, the spectrogram is not normalized.

For each time bin in the spectrogram, the corresponding spectrum can be collected at block 915. At block 920, one or more variables can be determined for the time bin based on the spectrum and one or more group-distinguishing frequency signatures. For example, a variable can include a power at a select frequency identified in a signature. As another example, a variable can include a value of a component (e.g., determined by calculating a weighted sum of power values in the spectrum) that is defined in a signature. Thus, in some instances, block 920 includes projecting a spectrum onto a new basis. Blocks 915 and 920 can be performed for each time bin.

At block 925, group assignments are made based on the associated variable. In some instances, individual time bins are assigned. In some instances, collections of time bins (e.g., individual epochs) are assigned to groups. Assignment can be performed, e.g., by comparing the variable to a threshold (e.g., such that it is assigned to one group if the variable is below a threshold and another otherwise) or by using a clustering or modeling technique (e.g., a Gaussian Naïve Bayes classifier). In some instances, the assignment is constrained such that a given feature (e.g., time bin or time epoch) cannot be assigned to more than a specified number of groups. This number may, or may not (depending on the embodiment), be the same as a number of groups or states (both base and non-base states) used to determine one or more group-distinguishing frequency signatures. The assignments can be generic (e.g., such that a clustering analysis produces an assignment to one of five groups, without tying any group to a particular physiological significance) or state specific.

Further, at each time interval, a fragmentation value can be defined. The fragmentation value can include a temporal fragmentation value or a spectral fragmentation value. For the temporal fragmentation value, a temporal gradient of the spectrogram can be determined and divided into segments. The spectrogram can include a raw spectrogram and/or a spectrogram having been normalized 1, 2 or more times across time bins and/or across frequencies (e.g., a spectrogram first normalized across time bins and then across frequencies). A given segment can include a set of time bins, each of which can be associated with a vector (spanning a set of frequencies) of partial-derivative power values. For each frequency, a gradient frequency-specific variable can be defined based on the partial-derivative power values defined for any time bin in the time block and for the frequency. For example, the variable can be defined as a mean of the absolute values of the partial-derivative power values for the frequency. A fragmentation value can be defined as a frequency with a high or highest frequency-specific variable. A spectral fragmentation value can be similarly defined but can be based on a spectral gradient of the spectrogram.

### IV. Translating Biological Signals to Control Computer Operations

In some embodiments, biological signals of a subject are used to identify various operations associated with a computing device. For example, activation sequence of the biological signals (e.g., biological signals activated from a left hemisphere of the brain) can be used to determine various types of operations to be performed by the computing device. Rather than relying on directly translating complex biological signals to a particular operation, the use of activation sequence and corresponding intent-communication interfaces (e.g., the intent-communication interface 1100) can reduce potential errors and lead to an efficient performance of computer operations.

### A. Activation Sequence

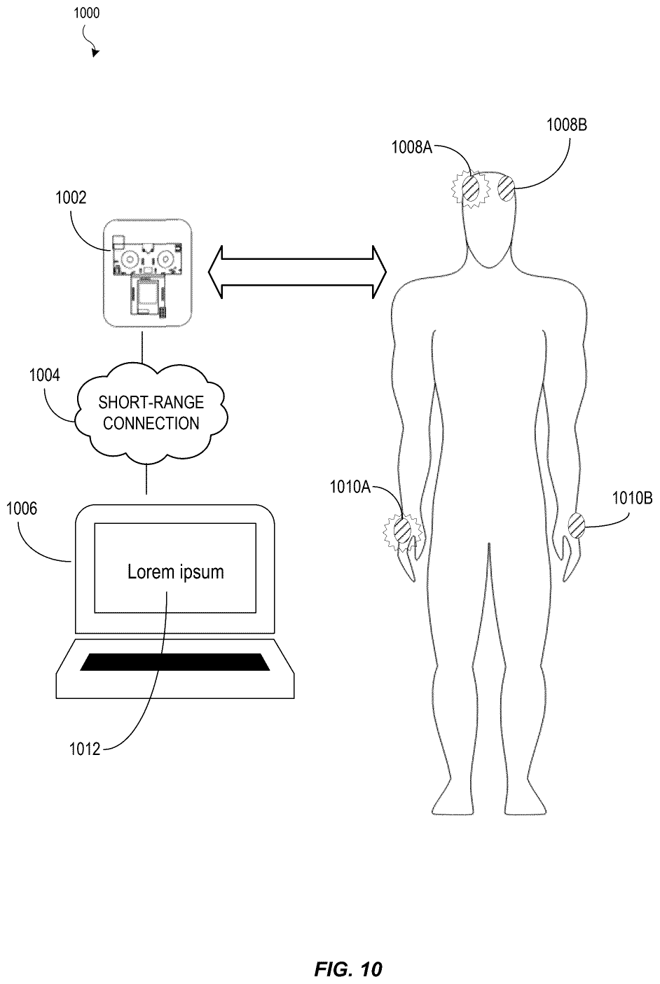

FIG. 10 illustrates a schematic diagram 1000 that shows an example of determining an activation sequence of biological signals, according to some embodiments. In some embodiments, a multi-electrode device 1002 accesses biological-signal data from a subject. The multi-electrode device 1002 (e.g., the multi-electrode device 110 of FIG. 1) can include software and hardware components for detecting and translating biological signals generated to move different portions of the body of the subject. For example, the multi-electrode device 1002 can include a housing having one or more clusters of electrodes. The biological-signal data collected by the multi-electrode device 1002 can include different types of biological signals. For example, the biological-signal data can include EEG data collected from electrodes placed on the subject's forehead. In another example, the biological-signal data can include EMG data collected from electrodes placed on the subject's limbs. In some instances, the biological-signal data are accessed by another computing device (e.g., the electronic device 120 of FIG. 1) via a wireless communication network (e.g., a short-range communication network). The biological-signal data can include the following data: (i) an indication of an intent to move a corresponding portion of a body; and (ii) a time point at which the biological signals were generated.

The biological signals from the subject can be analyzed to detect a signal-activation sequence. For example, detecting the signal-activation sequence can include processing the biological-signal data to identify a first signal and a second signal. The first signal represents an intent to move a first portion of the body of the subject. In some instances, the first signal was generated before the second signal. The second signal can represent another intent to move a second portion of the body of the subject. Thus, detecting the signal-activation sequence can include a determination that the first signals representing the intent to move the first portion of the body of the subject were generated before the second signals representing the other intent to move the second portion of the body of the subject. For example, the EEG data can indicate that biological signals detected from a right hemisphere of a brain 1008A of and representing an intent to move a left hand of the subject were generated before biological signals detected from a left hemisphere of the brain 1008B (e.g., left hemisphere of a brain) and representing another intent to move a right hand. In another example, the EMG data can indicate that biological signals representing an intent to move a portion 1010B (e.g., right hand) of the body were generated before biological signals representing another intent to move another portion 1010B (e.g., left arm) of the body. In some instances, different types of biological-signal data (e.g., EEG and EMG) are used together to determine or otherwise enhance the accuracy of determining the activation sequence of the biological signals.

The multi-electrode device 1002 can communicate, via short-range connection 1004, the signal-activation sequence of the biological signals to identify a particular operation to be performed by a computing device 1006. The operation may include inputting one or more alphanumerical characters on a graphical user interface of the computing device. In another example, the operation can include moving a cursor displayed by the graphical user interface. The operations can also include operations that are performed by different types of computing devices, including controlling one or more robot components or controlling augmented reality or virtual reality devices. The signal-processing application can then output instructions for the computing device to perform the identified operation. Continuing with the above example, the computing device 1006 can identify an operation to input the phrase “Lorem ipsum” 1012, in which each alphanumerical character can be determined and inputted based on utilizing the signal-activation sequence of the biological signals at a corresponding time point. As such, based on the activation sequence of biological signals, various types of operations can be performed to control the computing device 1006.

### B. Intent-Communication Interface

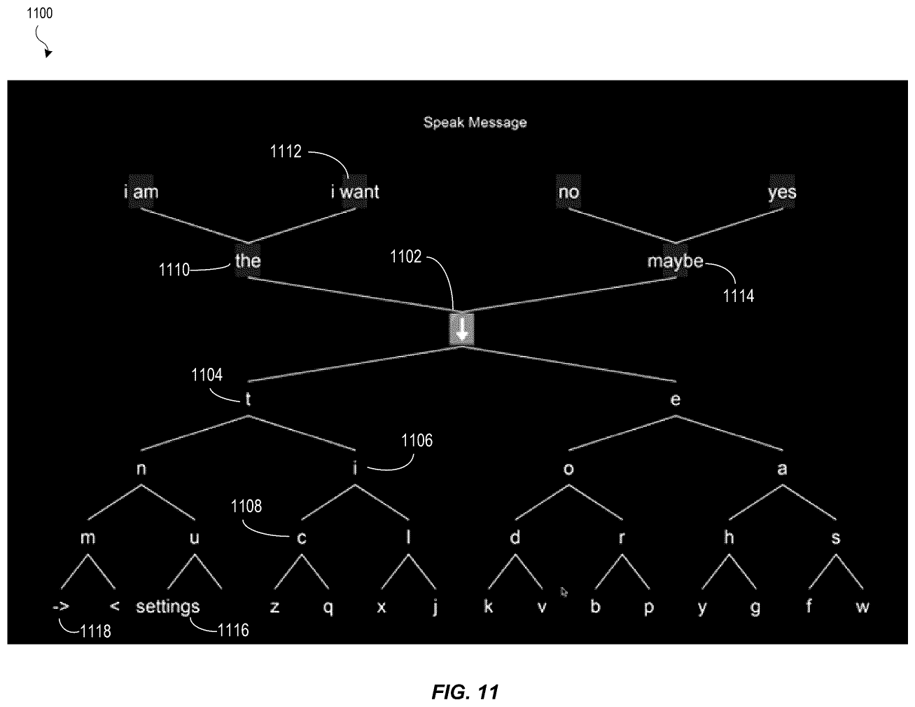

In some embodiments, biological-signal data are translated to access interface-operation data from one or more intent-communication interfaces, in which the interface-operation data is used by a signal-processing application to identify a particular operation to be performed by the computing device. In some instances, an intent-communication interface includes a set of interface elements, in which at least one interface element of the set includes a corresponding interface-operation data. FIG. 11 illustrates an example of an intent-communication interface 1100 used for translating biological-signal data to one or more computing-device operations, according to some embodiments. For example, the intent-communication interface 1100 can include a plurality of interface elements, in which each interface element of the plurality of interface elements of the intent-communication interface is connected with one or more children interface elements. Each interface element can include interface-operation data that identifies the particular operation, which can be accessed when the biological-signal data indicates an intention to simultaneously move both left and right portions of the body (e.g., an intent of squeezing both left and right hands together within a predetermined time interval). For example, a subject can access interface-operation data of a particular interface element of the intent-communication interface 1100 based on an intent of squeezing both left and right hands. The interface-operation data can be used by the same or another computing device to perform the particular operation.

In some instances, activation sequences of biological signals across a plurality of times are used to traverse one or more interface elements of the intent-communication interface, until a particular interface element is accessed and an associated operation is accessed. The traversal of the intent-communication interface 1100 can be initiated from a root interface element 1102 of the of the intent-communication interface 1100. For example, a cursor can be used to identify that the root interface element 1102 has been selected. The root interface element 1102 can be connected to one or more interface elements, at which biological-signal data at different time points can be processed. In FIG. 11, the root interface element 1102 is connected to four interface elements, including a “t” interface element, an “e” interface element, a “the” interface element, and a “maybe” interface element. In some instances, the root interface element 1102 includes interface-operation data that indicates a direction towards which the intent-communication interface 1100 is traversed. For example, the root interface element 1102 identifies a downward arrow, such that the intent-communication interface is traversed at a downward direction to access interface elements 1104, 1106, and 1108.

The subject can traverse the intent-communication interface 1100 based on an activation sequence of biological-signal data across a plurality of time points. For example, a multi-electrode device (e.g., the multi-electrode device 1002) can access biological-signal data from a subject at a first time point. The biological-signal data can be analyzed to determine that a first signal representing an intent to move a first portion of the body of the subject was generated, in which the first signal was generated before a second signal representing another intent to move a second portion of the body of the subject was generated. The first signal can then be translated to traverse the root interface element of the intent-communication interface to another interface element of the intent-communication interface. For example, the subject can imagine squeezing his left hand, which would result in biological-signal data being generated from a right hemisphere of the brain of the subject. The biological-signal data generated from the right hemisphere of the brain can be analyzed to determine that the intent-communication interface 1100 should be traversed from the root interface element 1102 to the “t” interface element 1104 (i.e., left child node). In some instances, the cursor identifies a selection of the interface element 1104. The subject can then either access the interface-operation data (e.g., input character “t” into a graphical-user interface) associated with the interface element 1104 based on an intent of squeezing both hands, or alternatively traverse the intent-communication interface 1100 based on an intent of squeezing his left hand or right hand.

Continuing with the example, the subject can continue traversing the intent-communication interface based on an intent of squeezing his right hand at a second time point. The intent of squeezing the right hand can be associated with biological signals being generated from a left hemisphere of the brain of the subject. The biological-signal data generated from the left hemisphere of the brain can be analyzed to determine that the intent-communication interface 1100 should be traversed from the “t” interface element 1104 to the “i” interface element 1106. After the second time point, the cursor can identify a selection of the interface element 1106. Similar to the previous instances, the subject can then either access the interface-operation data (e.g., input character “i” into a graphical-user interface) associated with the interface element 1106 based on the subject's intent of squeezing both hands, or further traverse the intent-communication interface 1100 based on an intent of squeezing his left hand or right hand instead. The above steps for traversing the intent-communication interface 1100 can be repeated across subsequent time points, until an interface element having the desired interface-operation data is reached. Continuing with this example, the traversal of the intent-communication interface 1100 can continue until the “c” interface element 1108 is reached at a third time point, at which the subject can access the interface-operation data (e.g., input character “c” into a graphical-user interface) associated with the interface element 1108 based on an intent of squeezing both hands. Various embodiments for inputting text and images using biological-signal data are also described in Section IV of the present disclosure.

The intent-communication interface 1100 can be applied or otherwise can enhance various operations associated with the computing device. In some embodiments, various types of data and operations are identified from the intent-communication interface 1110. As shown in FIG. 11, alphanumerical characters can be accessed from the interface elements 1104, 1106, and 1108. In addition, different words and phrases can be accessed from the intent-communication interface 1110. For example, a word “the” can be accessed from an interface element 1110, and a phrase “I want” can be accessed from an interface element 1112 of the intent-communication interface 1110. In some instances, if the biological-signal data representing an intent to move left or right portion of the body is detected at a leaf interface element (e.g., a node of the tree that has zero child nodes), the traversal of the intent-communication interface 1100 returns to the root interface element 1102 since there are no further interface elements that can be traversed from the leaf interface element. For example, if the signal-processing application receives biological-signal data indicating an intent to move the left portion of the body at the leaf interface element 1112, a cursor associated with the intent-communication interface can return to the root interface element 1102. Returning to the root interface element allows the subject to re-navigate the intent-communication interface 1102.

In some instances, one or more words or phrases are assigned to one or more interface elements of the intent-communication interface 1110. The words or phrases can be determined based on previous user data, at which the one or more words or phrases can be assigned to respective interface elements of the intent-communication interface 1110. For example, the previous user data can be processed to determine that the word “maybe” is a frequently used word for a given word-processing application. Based on the determination, the intent-communication interface 1110 can be updated such that an interface element 1114 includes the word “maybe.” In some instances, the previous user data includes user-specific data, such as document files created and edited by the subject. Additionally or alternatively, the previous user data can include user-population-specific data (e.g., similar geographic location, similar professions) and/or general-user data. Additionally or alternatively, one or more words or phrases can be configured by the user to be included into a default layout of the intent-communication interface 1110. For example, the word “please” is a frequently used term that can be configured by the user to be assigned to one of the interface elements of the intent-communication interface 1110, such that the word “please” will be displayed every time the intent-communication interface 1110 is availed to the user.

Additionally or alternatively, the interface elements of the intent-communication interface 1110 can identify one or more words or phrases predicted by a machine-learning model. For example, text data previously inputted on the graphical user interface include “the teacher typed into his computer . . . ”. Based on the inputted text data, one or more interface elements of the intent-communication interface 1110 can be updated to include predicted words or phrases that logically follow the existing text. Continuing with this example, an interface element can include one of the predicted words or phrases such as “keyboard”, “screen”, or “device”, in which the words and phrases are predicted by processing the previous text data using the machine-learning model (e.g., a long short-term memory neural network). Various embodiments for using machine-learning techniques for identifying the one or more words or phrases are described in Section V of the present disclosure.

In some embodiments, the intent-communication interface 1100 includes interface elements that identify operations for controlling the intent-communication interface 1110. For example, the subject can access the interface-operation data of the root interface element 1102 to trigger a change in direction towards which the intent-communication interface 1100 is traversed. For example, the subject can access, at a first time point, the interface-operation data (e.g., a downward arrow) associated with the root interface element 1102 based on an intent of squeezing both hands. The interface-operation data accessed from the root interface element 1102 can trigger a change from the downward arrow into an upward arrow. As a result, the intent-communication interface 1100 can be traversed through an upward direction, thereby enabling access of different characters or words associated with the interface element 1110 (“the”) and the interface element 1112 (the phrase “I want”). In another example, the subject can access interface-operation data from a particular interface element to access different data from the intent-communication interface 1110, including alphanumerical characters associated with a different language (e.g., German, Spanish) or a different set of frequently-used words or phrases. In some instances, accessing the different data from the intent-communication interface 1110 includes accessing an option to assign one or more words/phrases to corresponding interface elements, such that the corresponding interface elements become a part of a default layout of the intent-communication interface 1110. Thus, availing different configurations for the intent-communication interface 1110 can facilitate convenient access of various types of information from the intent-communication interface 1110.

In some embodiments, the intent-communication interface 1110 includes interface elements that identify functions associated with a particular application. The functions can be used to launch an application stored in the computing device or execute one or more commands associated with the application. For example, an interface element 1116 identifies the word “settings”, which is used as an application function for opening a settings menu of a word-processing application. In another example, an interface element 1118 identifies a “->” character, which is used as an application function for moving an insertion point of the word-processing application to a different location of the document. In some instances, some of the application functions are assigned to the corresponding interface elements based on previous user data.

### C. Methods for Translating Biological Signals to Control Computer Operations

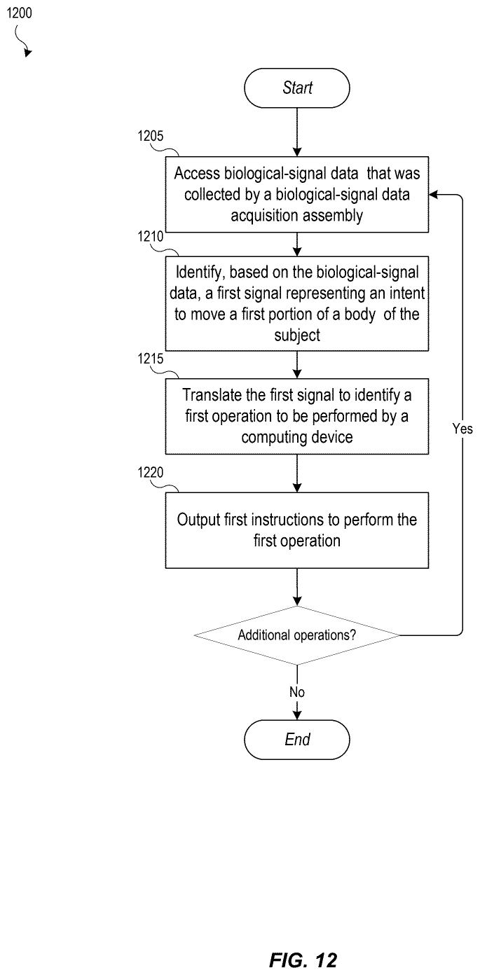

FIG. 12 illustrates a process 1200 for translating biological-signal data to one or more computing-device operations, in accordance with some embodiments. For illustrative purposes, the process 1200 is described with reference to the components illustrated in FIGS. 1-5, though other implementations are possible. For example, the program code stored in a non-transitory computer-readable medium is executed by one or more processing devices (e.g., the multi-electrode device 300 of FIG. 3, the electronic device 302 of FIG. 3) to cause the one or more processing devices to perform one or more operations described herein.

At step 1205, a signal-processing application accesses biological-signal data that was collected by a biological-signal data acquisition assembly. The biological-signal data acquisition assembly can include a housing having one or more clusters of electrodes, in which each cluster of the one or more clusters of electrodes comprises at least an active electrode. In some instances, the biological-signal data includes EEG data and/or EMG data.

At step 1210, the signal-processing application identifies a first signal representing an intent to move a first portion of a body of the subject based on the biological-signal data. In some instances, the first signal is generated before a second signal that represents another intent to move a second portion of the body of the subject. In some instances, if the biological-signal data includes the EEG data, the first signal is detected from a left hemisphere of a brain of the subject and the second signal is detected from a right hemisphere of the brain. The first portion can correspond to a left limb of the subject and the second portion can correspond to a right limb of the subject. The movement can include any type of action (e.g., squeezing, holding, shaking) associated with a corresponding portion of the body. In some instances, both of the EEG and the EMG data are used together to determine that the first signal was generated before the second signal.

At step 1215, the signal-processing application translates the first signal to identify a first operation to be performed by a computing device. In some instances, the first operation includes performing one or more functions associated with a graphical user interface of the computing device. The one or more functions associated with the graphical user interface can include: (i) moving a cursor displayed on the graphical user interface from a first location to a second location; (ii) inputting text onto the graphical user interface; and (iii) inputting one or more images or icons on the graphical user interface. In some instances, one or more machine-learning models are applied to the inputted text to predict additional text to be inputted onto the graphical user interface.

In some instances, the first operation includes launching an application stored in the computing device or executing one or more commands associated with the application. Additionally or alternatively, the first operation can be used to control various types of devices. For example, the computing device can be an augmented reality or virtual reality device, and the first operation can include performing one or more operations associated with the augmented reality or virtual reality device. In another example, the computing device can include one or more robotic components, in which the first operation includes controlling the one or more robotic components.

In some embodiments, the first operation includes accessing interface-operation data from an intent-communication interface, in which the interface-operation data is used to determine another operation to be performed by the computing device. In some instances, an intent-communication interface includes a set of interface elements. At least one interface element of the set can include a corresponding interface-operation data. For example, the intent- communication interface can be a tree that includes a root interface element connected to the first interface element and the second interface element. The first operation can include, from the root interface element, selecting a first interface element over a second interface element of the intent-communication interface. In some instances, the first interface element is associated with a first interface-operation data and a second interface element is associated with a second interface-operation data. A second operation to be performed by the computing device can then be identified by accessing the first interface-operation data of selected the first interface element. In some instances, the second operation is identified and selected when biological-signal data at a subsequent time point indicates an intent to simultaneously move both left and right portions of the body (e.g., squeezing both hands within a predetermined time interval). Second instructions to perform the second operation can then be outputted.

Additionally or alternatively, the intent-communication interface can be traversed to access other interface elements based on additional biological-signal data collected from the subject at subsequent time points. For example, additional biological-signal data collected by the biological-signal data acquisition assembly can be accessed at another time point. Based on the additional biological-signal data, a third signal representing a third intent to move the second portion of a body (e.g., biological signal representing an intent to move the left arm and detected from the right hemisphere of the brain) of the subject can be identified. In some instances, the third signal is generated before a fourth signal representing a fourth intent to move from the first portion (e.g., biological signal representing an intent to move of the right arm and detected from the left hemisphere of the brain) of the body of the subject. The third signal can then be translated to identify a third operation to be performed by a computing device.

Based on the third operation, a third interface element can be selected over a fourth interface element of the intent-communication interface, in which the third interface element and the fourth interface element are connected to the first interface element. In some instances, the third interface element is associated with a third interface-operation data and a fourth interface element is associated with a fourth interface-operation data. For example, the third interface element can be an interface element that includes the “c” character, and the fourth interface element can be another interface element that includes the “l” character. A fourth operation to be performed by the computing device can be identified by accessing the third interface-operation data of the selected third interface element. Continuing with the example, the fourth operation can include inputting the “c” character on a graphical user interface. After the fourth operation is identified, third instructions to perform the fourth operation can be outputted.

At step 1220, the signal-processing application outputs first instructions to perform the first operation. In some instances, the signal-processing application is internal to the computing device, in which the computing device can directly access the instructions and perform the operation. In some embodiments, the signal-processing application is external to the computing device. For example, the signal-processing application can be a part of an interface system (e.g., the multi-electrode device), in which the signal-processing application can transmit, over a communication network, the instructions to the computing device to perform the operation. Additionally or alternatively, the signal-processing application can transmit instructions to one or more accessory devices (e.g., smartwatch) communicatively coupled to the computing device, such that the one or more accessory devices can perform the identified operation. Additionally or alternatively, steps 1205 to 1220 can be repeated to perform multiple operations across a plurality of time points. Process 1200 terminates thereafter.

### V. Inputting Text and Images Using Biological Signals of a Subject

As described in Section III of the present disclosure, biological-signal data can be used by a signal-processing application to identify various operations to be performed by the computing device. An intent-communication interface can be used to facilitate brain-based communication of the subject by translating biological signals of the subject into one or more operations, such as inputting words or phrases into a word-processing application.

To navigate the intent-communication interface, biological signals that identify an intent to move a portion of the subject's body (e.g., left hand, right hand) can be used, regardless of whether an actual physical movement occurs. For example, if a subject desires to traverse the intent-communication interface towards a left interface element, the subject can imagine to squeeze his left hand. To access interface-operation data from the interface element, the subject can imagine to squeeze both hands at once. The configuration the intent-communication interface allows the subject to perform various computer operations without being constrained by a cursor speed. In some instances, alphanumerical characters are positioned in various interface elements of the intent-communication interface such that frequently used characters are positions closer to the root interface element of the intent-communication interface.

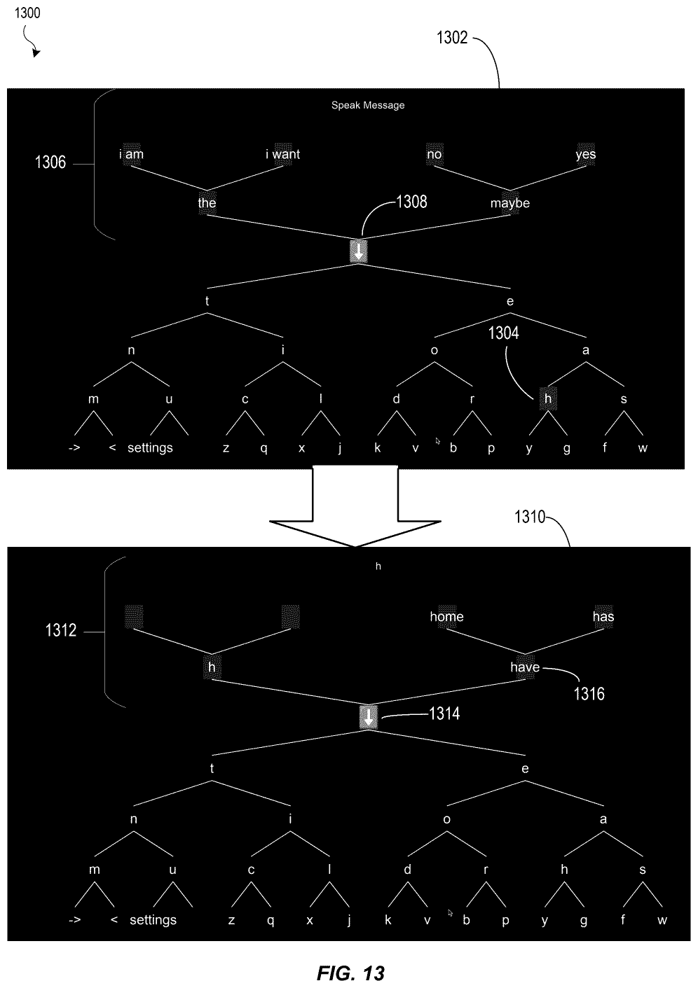

FIG. 13 illustrates an example schematic diagram 1300 of using an intent-communication interface for inputting text and images, according to some embodiments. In FIG. 13, an intent-communication interface 1302 includes a plurality of interface elements that respectively include interface-operation data that identifies the particular operation to be performed by a computing device. For example, the intent-communication interface 1302 includes an interface element 1304 that identifies an “h” character, as well as interface elements that respectively identify “t”, “e”, “n”, “i”, “o”, and “a” characters. In addition, the intent-communication interface 1302 also includes interface elements 1306 that respectively identify words or phrases, such as “the”, “i am”, “i want”, “no”, “maybe”, and “yes”. In some instances, the words or phrases above the intent-communication interface 1302 include recommended words or phrases that can be predicted based on previous user data, at which the one or more words or phrases can be assigned to respective interface elements of the intent-communication interface. In some instances, the recommended words or phrases include one or more phrases that complement the text that was previously inputted on the graphical user interface to form a complete sentence. For example, if the previously inputted text data includes “Please do not hesitate to . . . ”, a recommended phrase can include “contact us if you have any questions or comments.” The recommended phrase can be assigned to a corresponding interface element of the intent-communication interface. In some instances, the previous user data includes user-specific data, including document files created and edited by the subject. Additionally or alternatively, the previous user data can include user-population-specific data (e.g., similar geographic location, similar professions) and/or general-user data.

Additionally or alternatively, one or more words or phrases can be configured by the user to be included into a default layout of the intent-communication interface. For example, the word “please” is a frequently used term that can be configured by the user to be assigned to one of the interface elements of the intent-communication interface, such that the word “please” will be displayed every time the intent-communication interface is availed to the user.

The signal-processing application can translate the biological-signal data of the subject across different time points to traverse the intent-communication interface 1302 to a particular interface element (e.g., the “h” interface element 1304). In some instances, an activation sequence of biological signals is analyzed to determine which interface element of the intent-communication interface 1302 should be traversed. For example, the traversal begins at a root interface element 1308, at which a cursor can identify a selection of the root interface element 1308. The signal-processing application can detect a first biological-signal data generated at a first time point, in which the first biological-signal data represents an intent to move a first portion of a body (e.g., intent to squeeze the right hand). The signal-processing application can then traverse from the root interface element 1308 to the “e” interface element. The cursor can then identify that the “e” interface element has been selected. The signal-processing application can detect a second biological-signal data generated at a second time point, in which the second biological-signal data represents another intent to move the first portion of the body, thereby traversing from the “e” interface element to the “a” interface element. Finally, the signal-processing application can detect a third biological-signal data generated at a third time point, in which the third biological-signal data represents a third intent to move a second portion of the body (e.g., intent to squeeze the left hand). The signal-processing application can then traverse the intent-communication interface 1302 from the “a” interface element to the “h” interface element 1304. After the third time point, the cursor can identify that the “h” interface element 1304 has been selected.

At the “h” interface element 1302, the signal-processing application can access and input the “h” character to a graphical user interface (e.g., a word-processing application) if a fourth biological-signal data generated at a fourth time point is detected, in which the fourth biological-signal data represents a fourth intent to simultaneously move both first and second portions of the body. For example, a subject can access interface-operation data of the “h” interface element 1304 of the intent-communication interface 1302 based on an intent of squeezing both left and right hands simultaneously.

In some instances, if biological-signal data representing an intent to move left or right portion of the body is detected at a leaf interface element (e.g., a node of the tree that has zero child nodes), the traversal of the intent-communication interface 1302 returns to the root interface element 1308 since there are no further interface elements that can be traversed from the leaf interface element. For example, if the signal-processing application receives biological-signal data indicating an intent to move the first portion of the body at a given leaf interface element (e.g., “g” interface element), a cursor associated with the intent-communication interface can return to the root interface element 1308. Returning to the root interface element allows the subject to re-navigate the intent-communication interface 1302.

As shown in interface elements 1306, different words and phrases can be accessed from the intent-communication interface 1302. In some embodiments, one or more of the interface elements 1306 are updated based on words or characters that were previously inputted on the graphical user interface. Continuing with the example, once the “h” character is inputted on the word-processing application, the signal-processing application can modify a layout of the intent-communication interface 1302 to generate an updated intent-communication interface 1310. A layout of the updated intent-communication interface 1310 can include the same interface-operation data for interface elements that identify single alphanumerical characters. However, because the “h” character has been inputted, the updated intent-communication interface 1310 includes interface elements that respectively identify words such as “have”, “home”, and “has”. The subject can then traverse the updated intent-communication interface 1310 to input a completed word beginning with the letter “h”, thereby increasing efficiency of inputting text or images into the graphical user interface.

As an example implementation of inputting the word “have” into the word-processing application, the signal-processing application can initiate the traversal process at a root interface element 1314. The downward arrow at the root interface element 1314 can be modified to an upward arrow (not shown) based on detecting biological-signal data that represent an intent to simultaneous move the first and second portions of the body (e.g., intent to squeeze both hands at the same time). The upward arrow can indicate that the traversal of the updated intent-communication interface 1302 will be performed on an upward direction. At a subsequent timepoint, the signal-processing application can detect biological-signal data that is generated from the left hemisphere of the brain of the subject and represents another intent to move the first portion of the body (e.g., intent to squeeze the right hand). The signal-processing application can then traverse from the root interface element 1314 to the “have” interface element 1316. At the interface element 1316, the subject can input the word “have” into the word-processing application based on detecting yet another biological-signal data that represent an intent to simultaneous move the first and second portions of the body.

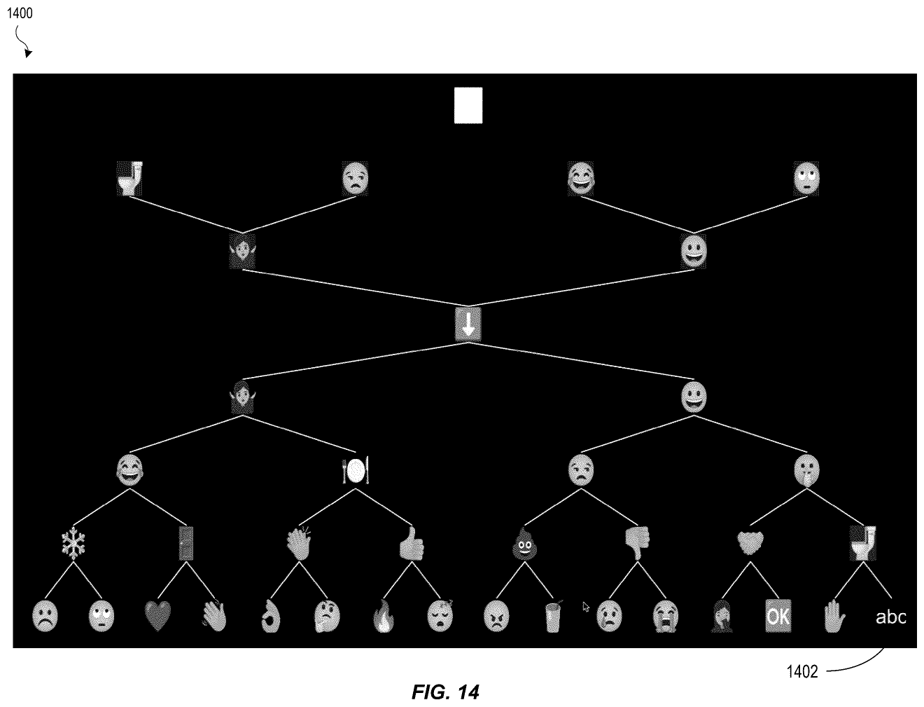

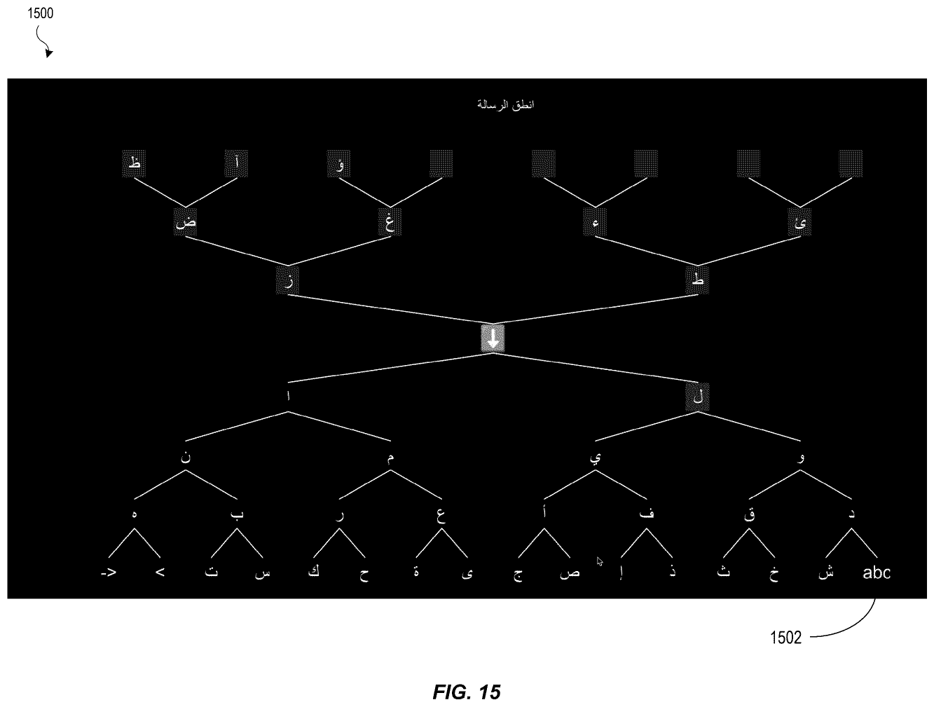

Additionally or alternatively, the intent-communication interface can be configured to provide other types of input, including images, emojis, and/or letters of other languages (e.g., Arabic). In some instances, various keyboard layouts are accessed from the intent-communication interface. In some instances, accessing the other types of input from the intent-communication interface includes accessing an option to assign one or more words/phrases to corresponding interface elements, such that the corresponding interface elements become a part of a default layout of the intent-communication interface. For example, FIG. 14 depicts an example of an intent-communication interface 1400 for inputting images, according to some embodiments. For example, an image inputted by the intent-communication interface 1400 can be an emoji. An emoji layout can be accessed instead of the English language layout by accessing an interface element (e.g., “settings” interface element 1116 of FIG. 11) that identifies an operation to switch from the English language layout to the emoji layout. In addition, the emoji layout of the intent-communication interface 1400 can be reverted back into the English language layout by accessing interface-operation data of an interface element 1402, which identifies another operation to switch back to the English language. In another example, FIG. 15 depicts another example of an intent-communication interface 1500 for inputting text of other languages, according to some embodiments. In FIG. 15, the intent-communication interface 1500 shows a layout that identifies characters of Arabic language. Similar to the emoji layout of the intent-communication interface 1400, the Arabic language layout can be reverted back into the English language layout by accessing interface-operation data of an interface element 1502, which identifies another operation to switch back to the English language.

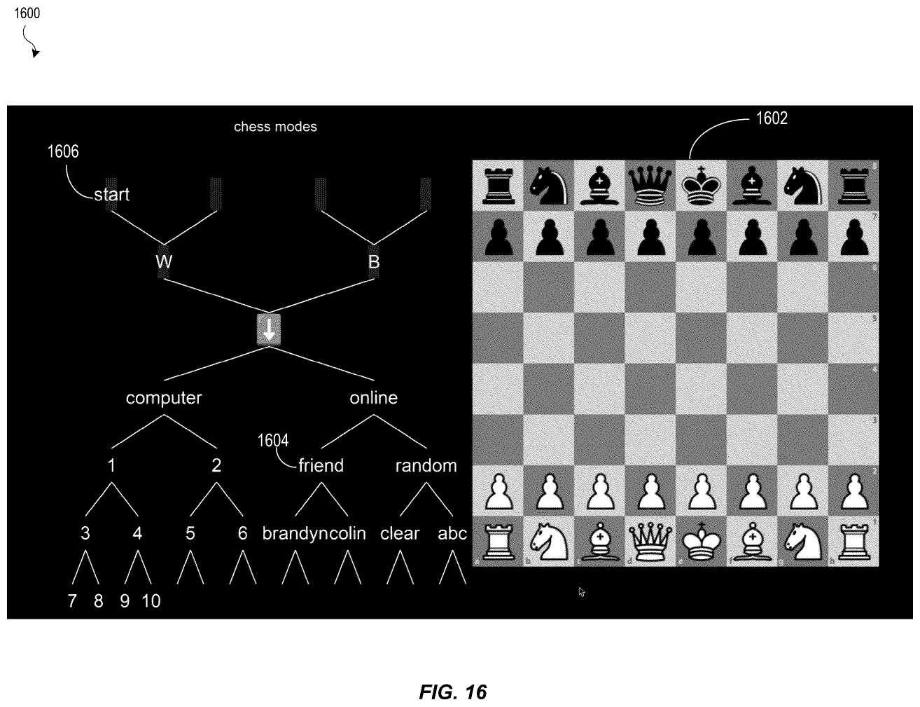

In some embodiments, the intent-communication interface is used to perform one or more operations associated with a particular type of application. The operations can be used to launch an application stored in the computing device or execute one or more commands associated with the application. For example, FIG. 16 depicts an example of an intent-communication interface 1600 for operating a computer application, according to some embodiments. The intent-communication interface 1600 can be used to perform one or more operations associated with a chess game application 1602. For example, biological-signal data of the subject across a first set of time points can be translated to select an option 1604 to play the chess game with a friend. Then, additional biological-signal data of the subject across a second set of time points can be translated to select an option 1606 to start the chess game. The layout of the intent-communication interface 1600 can then be updated to select and move the pieces of the chess game application 1602, which allows the subject to play the game without performing any physical movements.

### VI. Enhancing an Intent-Communication Interface Using Machine-Learning Techniques

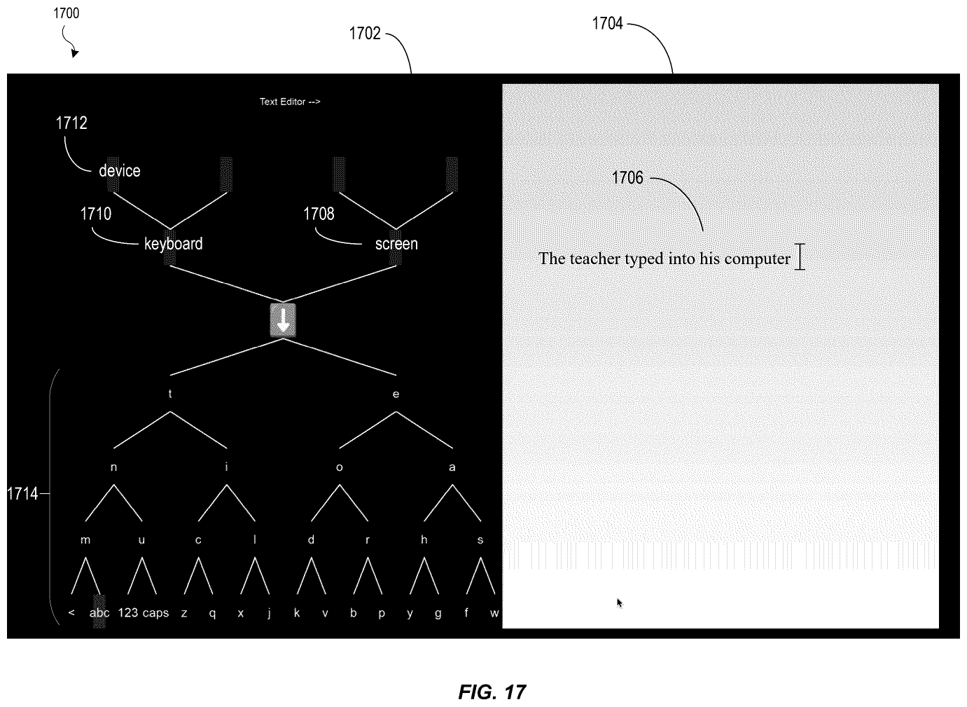

In addition, the intent-communication interface can be further enhanced by using machine-learning techniques. FIG. 17 depicts a schematic diagram 1700 of using machine-learning techniques to enhance an intent-communication interface, according to some embodiments. In FIG. 17, an intent-communication interface 1702 (e.g., the intent-communication interface 1302 of FIG. 13) is used to input words and phrases into a word-processing application 1704. The intent-communication interface 1702 can include one or more interface elements that identify words or phrases predicted by a machine-learning model. By populating the interface elements with words and phrases that are predicted based on context of existing text, the machine-learning techniques can increase efficiency of performing complex tasks on the graphical user interface. Additionally or alternatively, various operations corresponding to a particular type of application can be predicted then populated in the intent-communication interface 1702, as contemplated by one skilled in the art. For example, a machine-learning model can process an existing paragraph in the word-processing application and generate output predictive of text-formatting options such as “bold”, “italicize”, and “underline”.

As an illustrative example, the word-processing application 1704 displays text data 1706 inputted by the subject, which recite “the teacher typed into his computer . . . ”. A text-prediction application (not shown) can apply a machine-learning model to text data 1706, in which the machine-learning model was trained using training dataset that include text data previously inputted by the subject and/or other users. The machine-learning model can generate an output that includes one or more predicted words that would follow the text data 1706. For example, the predicted words may include “keyboard”, “screen”, or “device”. In some instances, the predicted words include one or more phrases that complement the text data to form a complete sentence. For example, if the previously inputted text data includes “Please do not hesitate to . . . ”, a predicted phrase can include “contact us if you have any questions or comments.” The predicted phrase can be assigned to a corresponding interface element of the intent-communication interface 1702. A layout of the intent-communication interface 1702 can be updated, such that at least some interface elements include the predicted words. In particular, one or more interface elements of the intent-communication interface 1702 can include the predicted words or phrases, such as a “screen” interface element 1708, a “keyboard” interface element 1710, and a “device” interface element 1712. In some instances, other interface elements 1714 of the intent-communication interface 1702 continue to include a set of default alphanumerical characters, to allow the user to input text that would be different from the predicted words or phrases.

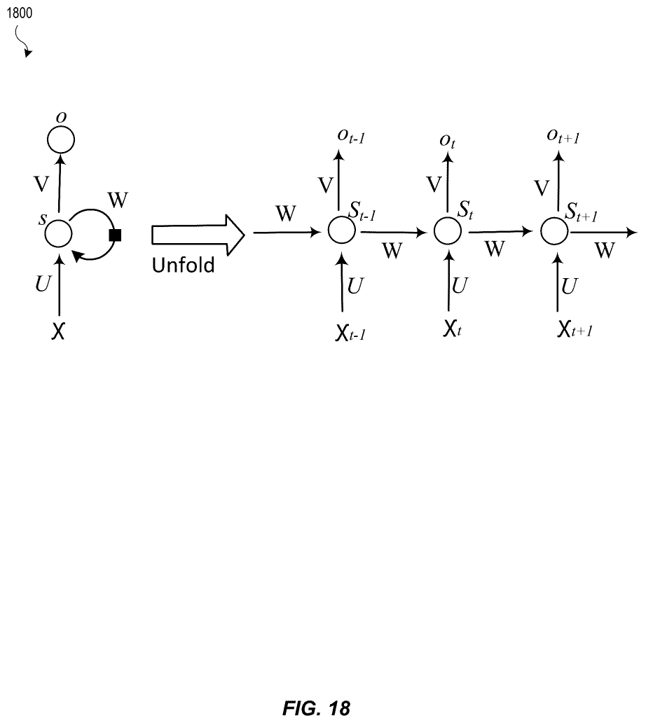

FIGS. 18-22 illustrate example configurations of a machine-learning model for predicting one or more words based on text data. To generate the predicted words, the text-prediction application can receive text data (e.g., the text data 1706) that includes a plurality of tokens (e.g., words, punctuation characters). The text-prediction application can preprocess the text data by encoding each token into an input embedding (e.g., a vector represented by a plurality of values) based on its semantic characteristics. In some instances, the text-prediction application is configured to generate input embedding with a predefined number of dimensions. Each input embedding can include a set of values that identify one or more semantic characteristics of the text data. In some instances, the text-prediction application uses a pre-trained model (e.g., word2vec, fastText) to encode each token into an input embedding.

The text-prediction application can apply a machine-learning model to the input embeddings that represent the text data. For example, the machine-learning model can be a recurrent neural network (RNN). Additionally or alternatively, the sequence-prediction layer includes a long short-term memory (LSTM) network, which is a type of an RNN. The LSTM network can be a bidirectional LSTM network. In some embodiments, the input embeddings are processed using one or more network layers of the machine-learning model to generate a set of output features. The set of output features can be processed using a fully-connected layer of the machine-learning model to generate an output that identifies one or more predicted words that follow the text data. As a result, the machine-learning model can generate the predicted words based on a contextual relationship between the words and the text data.

FIG. 18 depicts an example operation of the recurrent neural network 1800 for generating predicted words based on text data, according to some embodiments. As shown in RNNs include a chain of repeating modules (“cell”) of a neural network. Specifically, an operation of an RNN includes repeating a single cell indexed by a position of a text token (t) within the text tokens of the text data. In order to provide its recurrent behavior, an RNN maintains a hidden state sₜ, which is provided as input to the next iteration of the network. As referred herein, variables sₜ and hₜ are used interchangeably to represent a hidden state of the RNN. As shown in the left portion of FIG. 18, an RNN receives a feature representation for the text token xₜ and a hidden state value sₜ₋₁ determined using sets of input features of the previous text tokens. The following equation provides how the hidden state sₜ is determined:

$${s}_{t} = φ ( {Ux}_{t} + {Ws}_{t - 1} ) ,$$

where U and W are weight values applied to xₜ and sₜ₋₁ respectively, and φ is a non-linear function such as tanh or ReLU.

The output of the recurrent neural network is expressed as:

$${o}_{t} = soft max ( {Vs}_{t} ) ,$$

where V is a weight value applied to the hidden state value sₜ.

Thus, the hidden state sₜ can be referred to as the memory of the network. In other words, the hidden state sₜ depends from information associated with inputs and/or outputs used or otherwise derived from one or more previous text tokens. The output at step oₜ is a set of values used to generate one or more predicted words that follow the text data, which are calculated based at least in part on the memory at text token position t.

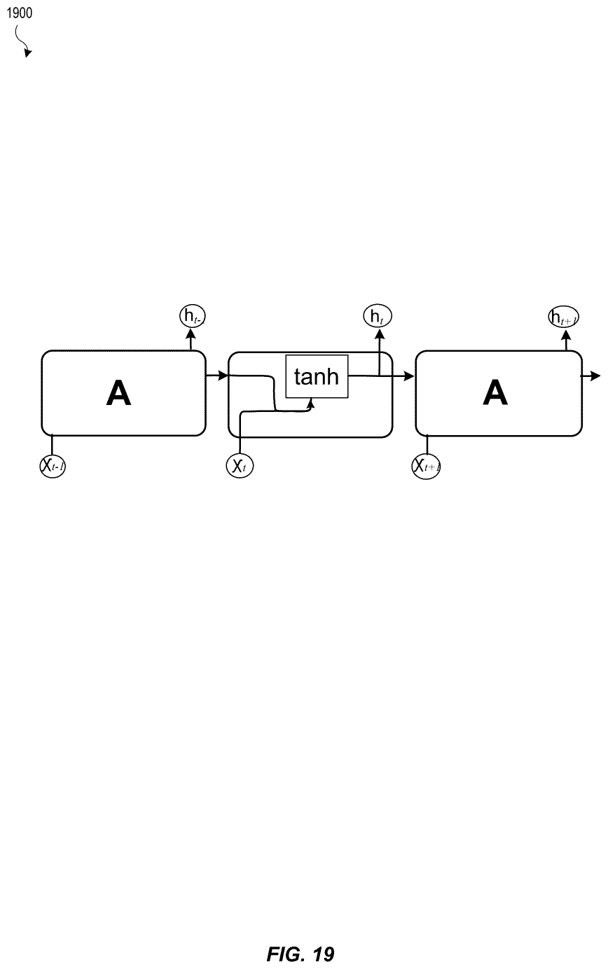

FIG. 19 illustrates another example of a recurrent neural network operation 1900 for generating predicted words based on text data, according to some embodiments. FIG. 19 depicts the RNN, in which the network has been unrolled for clarity. In FIG. 19, φ is specifically shown as the tanh function and the linear weights U, V and W are not explicitly shown. Unlike a traditional deep neural network, which uses different parameters at each layer, an RNN shares the same parameters (U, V, W above) across all text tokens. This reflects the fact that the same task is being performed at each text-section position, with different inputs. This greatly reduces the total number of parameters to be learned.

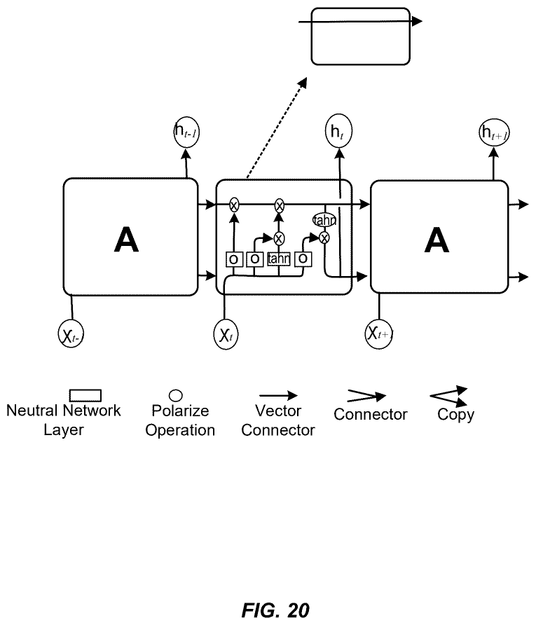

FIG. 20 depicts an example schematic diagram of a long short-term memory network 2000 for generating predicted words based on text data, according to some embodiments. An LSTM network is a type of an RNN, in which the LSTM network learns long-term dependencies between tokens of the text data. In some instances, the LSTM network is a bidirectional LSTM network. The bidirectional LSTM network applies two LSTM network layers to the input features of the text tokens: (i) a first LSTM network layer trained to process input features of the text tokens according to a forward sequence of text tokens in the text data (e.g., first text token to last text token); and (ii) a second LSTM network layer trained to process input features of the text tokens according to a reverse sequence of text tokens in the text data (e.g., last text token to first text token).

As shown in FIG. 20, an LSTM network may comprise a series of cells, similar to RNNs shown in FIGS. 18 and 19. Similar to an RNN, each cell in the LSTM network 2000 operates to compute a new hidden state for the next time step.

In addition to maintaining and updating a hidden state sₜ, the LSTM network maintains a cell state Cₜ. As used herein, a cell state encodes information of the inputs that have been observed up to that step (at every step). In some embodiments, rather than using a single layer for a standard RNN such as the tanh layer shown in FIG. 19, the LSTM network includes a second layer for adding and removing information from the cell via a set of gates. A gate includes a sigmoid function coupled to a pointwise or Hadamard product multiplication function, where the sigmoid function is:

$$σ = \frac{1}{1 + {e}^{- z}}$$

The ⊗ symbol or the ∘ symbol represents the Hadamard product. Gates can allow or disallow the flow of information through the cell. As the sigmoid function results in a value between 0 and 1, the functions value affects how much of each feature of a previous text token should be allowed through a gate. Referring again to FIG. 20, an LSTM network cell includes three gates: a forget gate; an input gate; and an output gate.

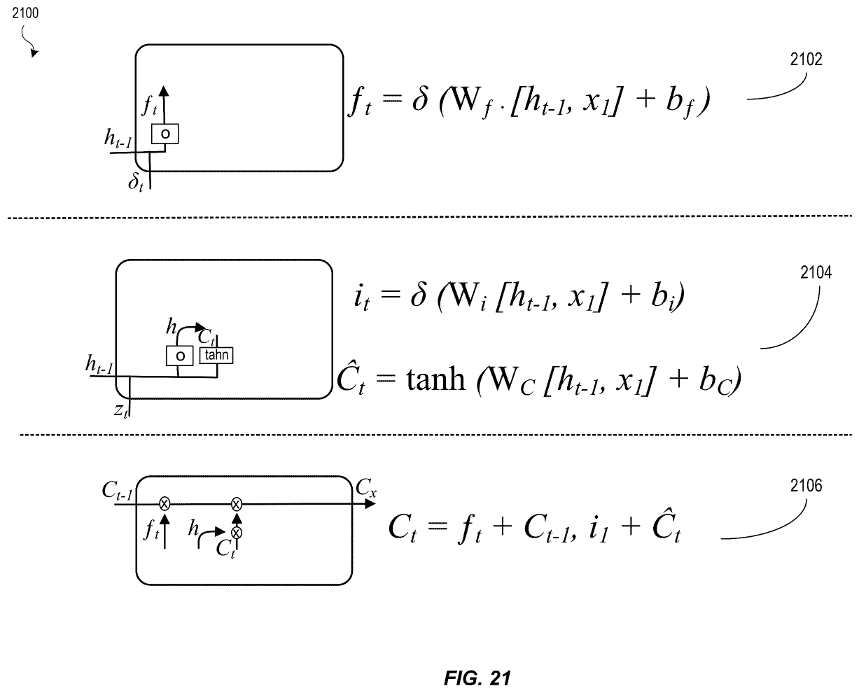

FIG. 21 illustrates an example schematic diagram 2100 for implementing forget and input gates of a long short-term memory network, according to some embodiments. For example, FIG. 21 illustrates a forget gate 2102 of an LSTM network. The LSTM network uses a forget gate to determine what information to discard in the cell state (long-term memory) based on the previous hidden state hₜ₋₁ and the current input xₜ. The LSTM network passes information from hₜ₋₁ and information from xₜ through a sigmoid function of the hidden gate. The output of the forget gate includes a value between 0 and 1. The LSTM network determines an output closer to 0 as information to forget. Conversely, the LSTM network determines an output closer to 1 as information to keep. An output value of the forget gate fₜ may be represented as:

$${f}_{t} = σ ( {W}_{f} [ {h}_{t - 1} , {x}_{t} ] + {b}_{f} ) ,$$

where W_{f} is a scalar constant, b_{f} is a bias term, and the brackets indicate concatenation of the input values.

FIG. 21 also depicts an operation of an input gate of a long short-term memory network, according to some embodiments. The LSTM network performs an input gate operation across two phases, which are shown respectively in phases 2104 and 2106. For example, a first phase 2104 of the LSTM network includes the LSTM network passing the previous hidden state and current input into a sigmoid function. The sigmoid function converts the input values (hₜ₋₁, xₜ) to determine whether the values of the cell state should be updated by transforming the input values a value between 0 and 1. In some instances, 0 indicates a value of less importance, and 1 indicates a value of more importance. In addition, the LSTM network passes the hidden state and current input into a tanh function to squish the input values between −1 and 1 to help regulate the network. The tanh function thus creates a vector of new candidate values \tilde{C}ₜ that may be added to the cell state. An output value of the sigmoid function it may be expressed by the following equation:

$${i}_{t} = σ ( {W}_{i} [ {h}_{t - 1} , {x}_{t} ] + {b}_{i} )$$

In addition, an output value of the tanh function \tilde{C}ₜ may be expressed by the following equation

$$\tilde{{C}_{t}} = tanh ( {W}_{c} [ {h}_{t - 1} , {x}_{t} ] + {b}_{c} )$$

A second phase 2106 can include multiplying the old state Cₜ₋₁ by the output value of the forget gate fₜ to facilitate forgetting of information corresponding to the input values to the forget gate. Thereafter, the new candidate values of the cell state iₜ⊗\tilde{C}ₜ are added to the previous cell state Cₜ₋₁ via pointwise addition. This may be expressed by the relation:

$${C}_{t} = {f}_{t} ⊗ {C}_{t - 1} + {i}_{t} ⊗ \tilde{{C}_{t}}$$

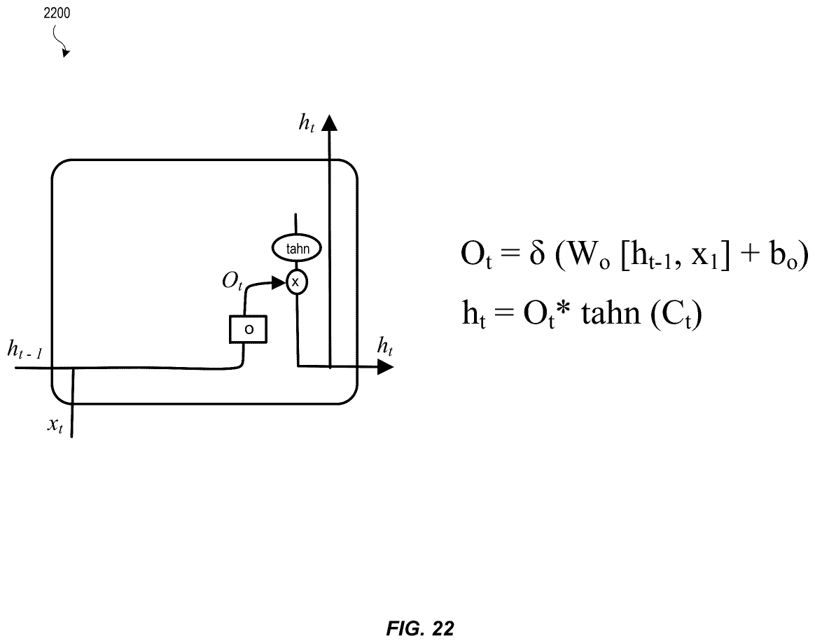

FIG. 22 depicts an example operation of an output gate 2200 of a long short-term memory network, according to some embodiments. The LSTM network uses the output gate to generate an output by applying a value corresponding to a cell state Cₜ. The output gate can be used to decide what the next hidden state should be. As described above, the hidden state can include information on previous inputs. The hidden state can also be used for predictions. First, the previous hidden state and the current input can be passed into a sigmoid function. Then, the newly modified cell state can be passed to the tanh function. The tanh output can be multiplied with the sigmoid output to determine what information the hidden state should carry. The output can thus be the hidden state. The new cell state and the new hidden can then carried over to the next time step.

For example, the LSTM network can pass the input values hₜ₋₁, xₜ to a sigmoid function. The LSTM network can apply a tanh function to a cell state Cₜ, which was modified by the forget gate and the input gate. The LSTM network can then multiply the output of the tanh function (e.g., a value between −1 and 1 that represents the cell state) with the output of the sigmoid function. The LSTM network can retrieve the hidden state determined from the output gate (e.g., return_sequence=true) and assign the hidden state as a set of output features used for generating the predicted words. For example, a fully connected neural network can be used to process a given output feature to generate the predicted words that follow the text data. The LSTM network may continue such retrieval process such that the set of output features are determined for the text tokens. In some instances, the output of the output gate is a new hidden state that is to be used for a subsequent text token of the text data. The operations of an output gate can be expressed by the following equations:

$${o}_{t} = σ ( {W}_{o} [ {h}_{t - 1} , {x}_{t} ] + {b}_{o} )$$

$${h}_{t} = {o}_{t} ⊗ tanh ( {C}_{t} )$$

The LSTM network as depicted in FIGS. 20-22 is only one example of a machine-learning model that uses the text data to generate predicted words or phrases. In some instances, a gated recurrent unit (“GRU”) is used or some other variant of an RNN. In addition, one ordinarily skilled in the art will recognize that the internal structures as shown in FIGS. 20-22 can be modified in a multitude of ways, for example, to include peephole connections.

### VII. Controlling Various Devices Based on Biological Signals of a Subject

In some embodiments, the intent-communication interface is used to perform operations associated with specific types of computing devices, including augmented or virtual reality devices, robotic components, and accessory devices. For example, augmented reality (AR) glasses can display a set of virtual screens. The intent-communication interface can be traversed using biological signals across different time points to select a first virtual screen of the set of virtual screen. Once the first virtual screen is selected, the interface elements of the intent-communication interface (e.g., modifying the layout of the intent-communication interface) can be automatically updated to include a set of operations (e.g., delete, create a new virtual screen, move to a different location, increase or decrease screen size, modify orientation of the screen). The intent-communication interface can then be traversed again to identify a particular operation (e.g., increase screen size) from the set of operations. The intent-communication interface can again be automatically updated such that the interface elements identify a subset of operations relating the increasing the screen size (e.g., 1x, 2x, 3x). As a result, multiple traversals of the intent-communication interface can be performed to efficiently perform tasks that are specifically associated with the AR glasses. The techniques for using activation sequence of biological signals can be extended to other types of devices, such as computing devices with robotic components (e.g., a drone device).

### A. Augmented Reality or Virtual Reality Devices

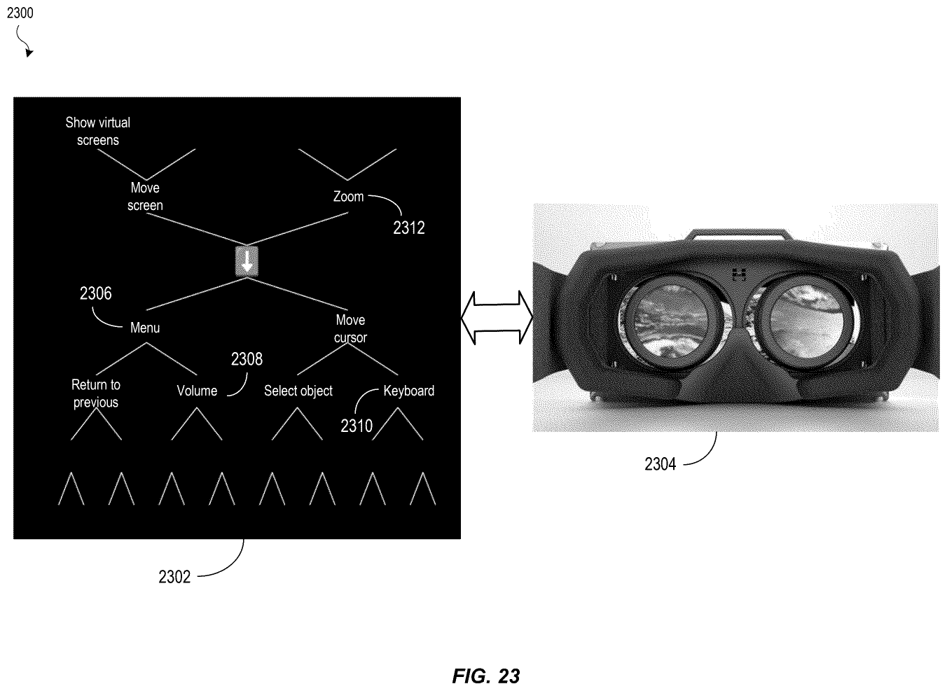

In some embodiments, biological-signal data are translated to access interface-operation data from one or more intent-communication interfaces, in which the interface-operation data is used by a signal-processing application to identify one or more operations to be performed by an augmented-reality or a virtual-reality device. FIG. 23 illustrates an example schematic diagram 2300 of an intent-communication interface 2302 for translating biological-signal data to one or more operations associated with a virtual-reality device 2304, according to some embodiments. For example, the intent-communication interface 2302 can include a plurality of interface elements. Each interface element can include interface-operation data that identifies the particular operation, which can be accessed by detecting biological-signal data that represent an intent to simultaneously move left and right portions of the body. For example, a subject can access interface-operation data of a particular interface element of the intent-communication interface 2300 based on an intent of squeezing both left and right hands.

As explained above, activation sequences of biological signals across a plurality of times can be used to traverse one or more interface elements of the intent-communication interface, until a particular interface element is accessed and an associated operation is accessed. For example, a multi-electrode device (e.g., the multi-electrode device 1002) can access biological-signal data from a subject at a first time point. The biological-signal data can be analyzed to detect a first signal that represents an intent to move the first portion of the body of the subject, in which the first signal was generated before a second signal that represents another intent to move a second portion of the body of the subject. The first signal can then be translated to traverse the root interface element of the intent-communication interface 2302 to another interface element of the intent-communication interface.

For example, the subject can imagine squeezing his left hand, which would result in detecting biological-signal data that is generated from a right hemisphere of the brain of the subject. The biological-signal data generated from the right hemisphere of the brain can be analyzed to determine that the intent-communication interface 2302 should be traversed from the root interface element to the “Menu” interface element 2306. In some instances, the cursor identifies a selection of the “Menu” interface element 2306. The subject can then access the interface-operation data associated with the interface element 2306 based on an intent of squeezing both hands. The “Menu” operation can then be performed by the virtual-reality device 2304, which may result in a separate virtual screen with different menu options being displayed on the virtual-reality device 2304. In some instances, a layout of the intent-communication interface 2302 is modified to include a set of sub-operations that can be performed by the virtual-reality device 2304, in which the set of sub-operations include one or more operations that can be performed within the “Menu” (e.g., open a game or chat application, configure wireless network settings).

Alternatively, instead of accessing the “Menu” interface element 2306, the subject can further traverse the intent-communication interface 2302 based on an intent of squeezing his right hand, which results in reaching a “Volume” interface element 2308. The subject may access interface-operation data associated with the “Volume” interface element 2308, which triggers a modification of the layout of the intent-communication interface 2302 to include a “+” interface element for increasing the volume of the virtual-reality device 2304 and a “−” interface element for decreasing the volume of the virtual-reality device 2304. The subject can then traverse the modified intent-communication interface 2302 to increase or decrease the volume of the virtual-reality device 2304.

Various types of operations associated with the virtual-reality device 2304 can populate the interface elements of the intent-communication interface 2302. For example, a “Keyboard” interface element 2310 can be accessed to perform displaying a modified intent-communication interface with a layout that includes alphanumerical characters and predicted words (e.g., the intent-communication interface 1100 at FIG. 11). The subject can also access the interface-operation data of the root interface element of the intent-communication interface 2302 to trigger a change in direction towards which the intent-communication interface 2302 is traversed. For example, the intent-communication interface 2300 can be traversed through an upward direction, thereby allowing access to interface-operation data of a “Zoom” interface element 2312. The interface-operation data of the “Zoom” interface element 2312 can be used to change a zoom level of one or more image objects that are being displayed on the accessing interface-operation data of a “Zoom” interface element 2312. One skilled in the art can populate the interface elements of the intent-communication interface 2302 with other types of operations associated with the virtual-reality device 2304, which facilitates efficient control of the virtual- reality device 2304 based on biological signals of the subject (and without any physical movements).

### B. Robot Devices

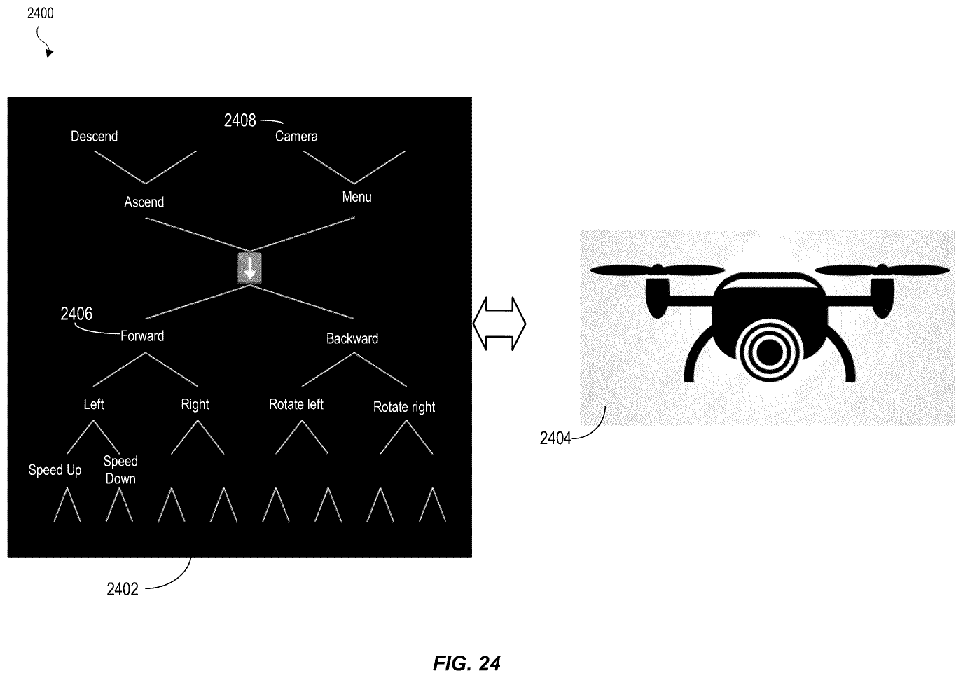

In some embodiments, biological-signal data are translated to access interface-operation data from one or more intent-communication interfaces, in which the interface-operation data is used by a signal-processing application to identify one or more operations to be performed by a computing device with one or more robotic components. FIG. 24 illustrates an example schematic diagram 2400 of using an intent-communication interface 2402 for translating biological-signal data to one or more operations associated with a computing device with one or more robotic components, according to some embodiments. The robot components can be associated with any robot type (e.g., a humanoid robot, an assembly line robot). For example, the computing device can be a drone device 2404 that includes components for flying in the air. For example, the intent-communication interface 2402 can include a plurality of interface elements. Each interface element can include interface-operation data that identifies the particular operation, which can be accessed when the biological-signal data representing an intent to simultaneously move left and right portions of the body is detected.

Activation sequences of biological signals across a plurality of times can thus be used to traverse one or more interface elements of the intent-communication interface, until a particular interface element is accessed and an associated operation is accessed. For example, a multi-electrode device (e.g., the multi-electrode device 1002) can access biological-signal data from a subject at a first time point. The biological-signal data can be analyzed to detect a first signal that represents an intent to move the first portion of the body of the subject, in which the first signal was generated before a second signal that represents another intent to move a second portion of the body of the subject. The first signal can then be translated to traverse the root interface element of the intent-communication interface 2402 to another interface element of the intent-communication interface.

For example, the subject can imagine squeezing his left hand, which would result in detecting biological-signal data that is generated from a right hemisphere of the brain of the subject. The biological-signal data generated from the right hemisphere of the brain can be analyzed to determine that the intent-communication interface 2402 should be traversed from the root interface element to the “Forward” interface element 2406. The subject can then access the interface-operation data associated with the interface element 2406 based on an intent of squeezing both hands. The “Forward” operation can then be performed by the drone device 2404, which may result in the drone device 2404 moving in a forward direction. In some instances, the cursor does not return to the root interface element but remains in the “Forward” interface element 2406 such that the drone device 2404 can continue to move in the forward direction. To return to the root interface element, the subject can traverse to a leaf interface element (e.g., a node of the tree that has zero child nodes). If biological-signal data representing an intent to move left or right portion of the body at the leaf interface element, the traversal of the intent-communication interface 2402 can return to the root interface element.

The subject can further traverse the intent-communication interface 2402 to access other types of operations, including a “Rotate left” operation, a “Menu” operation, an “Ascend” operation, and a “Descend” operation. In some instances, a camera component of the drone device 2404 is activated based on accessing interface-operation data associated with a “Camera” interface element 2408. One skilled in the art can populate the interface elements of the intent-communication interface 2402 with other types of operations associated with the drone device 2404, which facilitates efficient control of the drone device 2404 based on biological signals of the subject (and without requiring any physical movements).

### C. Accessory Devices

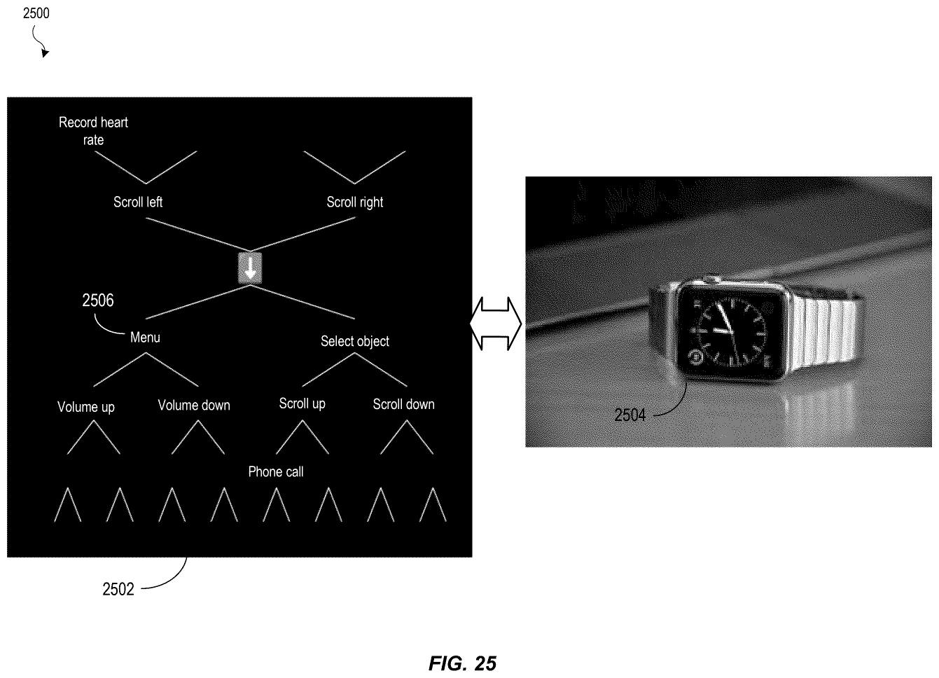

In some embodiments, biological-signal data are translated to access interface-operation data from one or more intent-communication interfaces, in which the interface-operation data is used by a signal-processing application to identify one or more operations to be performed by an accessory device. FIG. 25 illustrates an example schematic diagram 2500 of using an intent-communication interface 2502 for translating biological-signal data to one or more operations associated with an accessory device, according to some embodiments. The accessory device can include various types of devices (e.g., wireless headphones, a heart monitor, a smartwatch). For example, the accessory device can be a smartwatch device 2504. For example, the intent-communication interface 2502 can include a plurality of interface elements. Each interface element can include interface-operation data that identifies the particular operation, which can be accessed when the biological-signal data indicates that left and right portions of the body have been simultaneously activated (e.g., both portions activated within a predetermined time interval).

Activation sequences of biological signals across a plurality of times can be used to traverse one or more interface elements of the intent-communication interface, until a particular interface element is accessed and an associated operation is accessed. For example, a multi-electrode device (e.g., the multi-electrode device 1002) can access biological-signal data from a subject at a first time point. The biological-signal data can be analyzed to detect a first signal that represents an intent to move the first portion of the body of the subject, in which the first signal was generated before a second signal that represents another intent to move a second portion of the body of the subject. The first signal can then be translated to traverse the root interface element of the intent-communication interface 2502 to another interface element of the intent-communication interface.

For example, the subject can imagine squeezing his left hand, which would result in detecting biological-signal data that is generated from a right hemisphere of the brain of the subject. The biological-signal data generated from the right hemisphere of the brain can be analyzed to determine that the intent-communication interface 2502 should be traversed from the root interface element to the “Menu” interface element 2506. The subject can then access the interface-operation data associated with the interface element 2506 based on an intent of squeezing both hands. The “Menu” operation can then be performed by the smartwatch device 2504, which may result in displaying of different menu options on the smartwatch device 2504. In some instances, a layout of the intent-communication interface 2502 is modified to include a set of sub-operations that can be performed by the smartwatch device 2504, in which the set of sub-operations include one or more operations that can be performed within the “Menu” (e.g., open a smartwatch application, configure wireless network settings).

The subject can further traverse the intent-communication interface 2502 to access other types of operations, including a “Select object” operation, a “Scroll left” operation, an “Record heart rate” operation, and a “Volume up” operation. One skilled in the art can populate the interface elements of the intent-communication interface 2502 with other types of operations associated with the smartwatch device 2504, which facilitates efficient control of the smartwatch device 2504 based on biological signals of the subject (and without any physical movements).

### VIII. Example of a Computing Environment

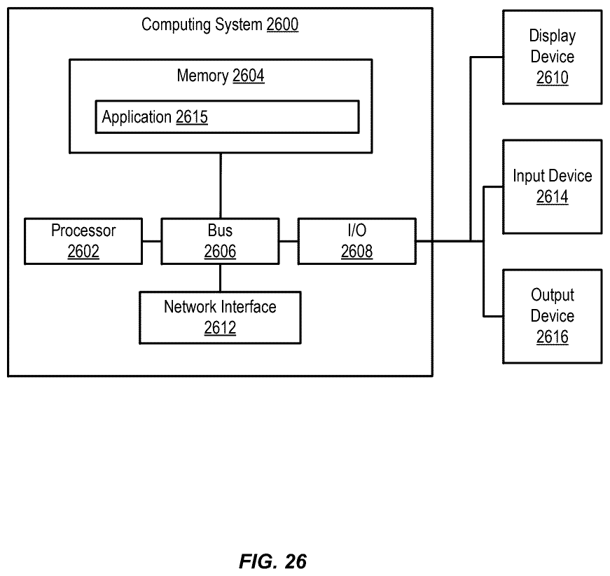

Any suitable computing system or group of computing systems can be used for performing the operations described herein. For example, FIG. 26 depicts a computing system 2600 that can implement any of the computing systems or environments discussed above. In some embodiments, the computing system 2600 includes a processing device 2602 that executes a signal-processing application 2615 for translating biological signals to computer-device operations, a memory that stores various data computed or used by the signal-processing application 2615, an input device 2614 (e.g., a mouse, a stylus, a touchpad, a touchscreen), and an output device 2616 that presents output to a user (e.g., a display device that displays graphical content generated by the signal-processing application 2615). For illustrative purposes, FIG. 26 depicts a single computing system on which the signal-processing application 2615 is executed, and the input device 2614 and output device 2616 are present. But these applications, datasets, and devices can be stored or included across different computing systems having devices similar to the devices depicted in FIG. 26.

The example of FIG. 26 includes a processing device 2602 communicatively coupled to one or more memory devices 2604. The processing device 2602 executes computer-executable program code stored in a memory device 2604, accesses information stored in the memory device 2604, or both. Examples of the processing device 2602 include a microprocessor, an application-specific integrated circuit (“ASIC”), a field-programmable gate array (“FPGA”), or any other suitable processing device. The processing device 2602 can include any number of processing devices, including a single processing device.

The memory device 2604 includes any suitable non-transitory, computer-readable medium for storing data, program code, or both. A computer-readable medium can include any electronic, optical, magnetic, or other storage device capable of providing a processor with computer-readable instructions or other program code. Non-limiting examples of a computer-readable medium include a magnetic disk, a memory chip, a ROM, a RAM, an ASIC, optical storage, magnetic tape or other magnetic storage, or any other medium from which a processing device can read instructions. The instructions may include processor-specific instructions generated by a compiler or an interpreter from code written in any suitable computer-programming language, including, for example, C, C++, C#, Visual Basic, Java, Python, Perl, JavaScript, and ActionScript.

The computing system 2600 may also include a number of external or internal devices, such as a display device 2610, or other input or output devices. For example, the computing system 2600 is shown with one or more input/output (“I/O”) interfaces 2608. An I/O interface 2608 can receive input from input devices or provide output to output devices. One or more buses 2606 are also included in the computing system 2600. Each bus 2606 communicatively couples one or more components of the computing system 2600 to each other or to an external component.

The computing system 2600 executes program code that configures the processing device 2602 to perform one or more of the operations described herein. The program code includes, for example, code implementing the signal-processing application 2615 or other suitable applications that perform one or more operations described herein. The program code may be resident in the memory device 2604 or any suitable computer-readable medium and may be executed by the processing device 2602 or any other suitable processor. In some embodiments, all modules in the signal-processing application 2615 are stored in the memory device 2604, as depicted in FIG. 26. In additional or alternative embodiments, one or more of these modules from the signal-processing application 2615 are stored in different memory devices of different computing systems.

In some embodiments, the computing system 2600 also includes a network interface device 2612. The network interface device 2612 includes any device or group of devices suitable for establishing a wired or wireless data connection to one or more data networks. Non-limiting examples of the network interface device 2612 include an Ethernet network adapter, a modem, and/or the like. The computing system 2600 is able to communicate with one or more other computing devices (e.g., a computing device that receives inputs for the signal-processing application 2615 or displays outputs of the signal-processing application 2615) via a data network using the network interface device 2612.

An input device 2614 can include any device or group of devices suitable for receiving visual, auditory, or other suitable input that controls or affects the operations of the processing device 2602. Non-limiting examples of the input device 2614 include a touchscreen, stylus, a mouse, a keyboard, a microphone, a separate mobile computing device, etc. An output device 2616 can include any device or group of devices suitable for providing visual, auditory, or other suitable sensory output. Non-limiting examples of the output device 2616 include a touchscreen, a monitor, a separate mobile computing device, etc.

Although FIG. 26 depicts the input device 2614 and the output device 2616 as being local to the computing device that executes the application for translating biological signals, other implementations are possible. For instance, in some embodiments, one or more of the input device 2614 and the output device 2616 include a remote client-computing device that communicates with the computing system 2600 via the network interface device 2612 using one or more data networks described herein.

### IX. Traumatic Brain Injury Prediction Based on Sleep States

Certain aspects and examples of the present disclosure relate to a system and method for predicting the presence of a traumatic brain injury (TBI) based on neural-signal data associated with one or more sleep states. The neural-signal data may be obtained over one or more sleep time periods for a subject via a physiological data acquisition assembly. The physiological data acquisition assembly includes at least a single channel of neural-signal data with at least one reference electrode and at least one active electrode in close proximity. The assembly can be worn by the subject. For example, the assembly can include a patch configurable to be positioned on (e.g., adhered to) the subject's forehead. Additionally, the patch can have an adhesive film to which the electrodes can be attached to collect the neural-signal data.

In some examples of the present disclosure, the neural-signal data can be used to predict, characterize, and/or analyze the one or more sleep states. The sleep states can be any distinguishable state of sleep or wakefulness that are representative of behavioral, physical, or signal characteristics. In some instances, neural-signal data is processed to infer—for each of multiple time intervals—a category that indicates a prediction as to whether the subject is awake or asleep, and potentially—if the subject is estimated as being asleep—a particular type or stage of sleep. The inference can be made based on—for each of multiple time intervals—transforming time-domain electrical signals into frequency-domain intensity or power values. Features may be defined as cumulative or maximum intensity or power values within various frequency bands. Sleep states may then be inferred based on absolute or relative values of one or more features. The states may include a Stage 1 sleep state, a Stage 2 sleep state, a Stage 3 sleep state, and a REM sleep state.

Moreover, in some examples, artificial-intelligence (AI) techniques can be used to predict that a subject has a given condition, to predict a severity of the given condition, or to predict an efficacy of treating the given condition. An AI technique may include implanting signal processing (e.g., that may include applying one or more signal transformations) and using one or more models or rules to generate an epoch-specific, night-specific or subject-specific prediction. For example, neural signals may be collected across a sleep time period (e.g., a night). The neural signals may be separated into epochs that correspond to absolute or relative time increments through the time period (e.g., 1-minute, 5-minute, or 10-minute time intervals), and a spectrum can be generated for each epoch, such that a power or intensity for each of various frequency bands may be identified for each time increment.

The presence of a TBI can be associated with reduced Stage 2 sleep. The presence of the TBI may further be associated with increased slow wave sleep (SWS) (i.e., Stage 3 sleep). Thus, an artificial-intelligence rule can be defined to predict Stage 2 sleep deprivation and/or likelihood of TBI based on the features. For example, a clustering technique, support vector machine (SVM) technique, principal components technique, independent components technique, logistic regression technique, etc. may be used to predict—for each time epoch—whether the subject is in Stage 2 sleep (versus Stage 1, Stage 3, REM, or awake). In some instances, for each epoch, a likelihood of the subject being in Stage 2 sleep is generated, which may then be compared against a predefined or learned threshold to predict whether the subject is or was in Stage 2 sleep.

Additionally, a rule can be defined to predict—based on the Stage 2 sleep predictions—whether the subject has a TBI. For example, the rule may indicate that the subject has a TBI if less than a threshold percentage (e.g., 10%, 15%, 20%, 25%, 30%, or 35%) of the epochs are predicted to be Stage 2 sleep. In another example, a rule may indicate that the subject has a TBI by identifying that a length of time (e.g., a relative or absolute length of time) for Stage 2 sleep for one or more epochs is less than a predefined threshold for healthy or normal sleep or for a learned threshold for the subject. Similarly, a rule may indicate that the subject has a TBI if more than a threshold percentage of the epochs are predicted to be Stage 3 sleep. Additionally, a rule may indicate that the subject has a TBI by identifying that a length of time for Stage 3 sleep is greater than a predefined threshold for healthy or normal sleep or for a learned threshold for the subject.

In a particular example, a TBI may be suspected for a subject following an injury to the head. In response, neural-signal data may be collected and processed for a night of sleep following the injury. The neural-signal data can be split into time segments and detection algorithms can be used to predict a subset of the time segments associated with Stage 2 sleep. Additionally, segment-specific metrics can be determined for each of the subset of time segments. The segment-specific metrics can be lengths of time for the Stage 2 sleep. Then, the segment-specific metrics can be combined to generate a cumulative metric representing an estimated absolute amount of time for which it is predicted that the subject was in Stage 2 sleep over the night of sleep. Moreover, the AI techniques can be implemented to generate a risk-level metric based on the cumulative metric. The risk-level metric can be a likelihood that the subject has a TBI. The AI techniques may output the risk-level metric based on, for example, a predefined rule that that indicates the risk-level metric based on the cumulative metric being less than one or more threshold lengths of time. For example, the AI techniques can learn the one or more threshold from data indicating normal or average lengths of time for Stage 2 sleep for a night of sleep for healthy subject or for the subject prior to the suspected TBI. The AI techniques can then be trained to output a percentage as the risk-level metric to represent the likelihood that the subject has a traumatic brain injury based on the cumulative metric and the learned thresholds.

In this way, detection and diagnosis of TBIs can be improved. In particular, by analyzing neural-signal data and implementing the detection algorithms to predict sleep states, metrics (e.g., absolute or relative amounts of time for which the subject was in a particular sleep state) can be derived with improved accuracy. Additionally, by implementing AI techniques to predict the risk-level metric based on, for example, a cumulation of the metrics, an accuracy of diagnosing TBIs can be improved. In particular, the risk-level metric can provide a more accurate representation of the likelihood that a subject has a TBI than neurological exams due to the AI techniques being trained to perform the predictions using previous sleep data for the subject or for associated subjects (e.g., healthy subjects of a similar age to the subject, subjects of the similar age with TBIs, etc.). The risk-level metric can also be more accurate than current imaging modalities for diagnosing TBIs, due to the changes in sleep patterns used for predicting the risk-level metric being associated with all levels of TBIs (i.e., mild, moderate, and severe TBIs). Additionally, monitoring the subject to generate the risk-level metric and outputting a result (e.g., a percentage representing the risk-level metric) can facilitate efficient treatment of TBIs.

### X. Sleep State Predictions

The neural-signal data collected over the one or more time periods via the physiological data acquisition assembly can be split into time segments. Typically, neural signals can be examined in time in series increments called epochs. For example, when the neural signals are used for analyzing sleep, sleep may be segmented into one or more epochs to use for analysis. The epochs can be segmented into different sections using a scanning window, where the scanning window defines different sections of the time series increment. Code can move (incrementally or via shifting) the scanning window via a sliding or shifting window, where sections of the sliding window have overlapping or non-overlapping time series sequences. An epoch can alternatively span an entire time series, for example. In some examples, each epoch can be classified to correspond to a predicted sleep state that is represented. In some instances, prior to the classification, the epoch is normalized or double normalized based on (for example) frequency information, amplitude information, power, intensity, or other suitable features of the EEG data that can be correlated with sleep states. U.S. patent application Ser. No. 11/431,425, filed on May 9, 2006, which is hereby incorporated by reference for all purposes, discloses exemplary techniques for normalizing biological data.

Additionally, to predict sleep states, detection algorithms may be configured in the time or frequency domain to detect signatures (e.g., frequency domain features, time domain features, time-frequency domain features, etc.) that support predictions as to whether the subject is asleep and, in the case that sleep is detected, the detection algorithms may further support predictions of sleep states. The detection algorithms can be performed with respect to one or more epochs to predicts sleep states for the one or more epochs. To illustrate, a wake sleep state can be predicted by detecting signals within one or more particular frequency bands (e.g., a band that extends between about thirteen and about sixty hertz (Hz) with amplitudes of at least about thirty microvolts (μV) (i.e., Beta waves). The frequency bands and amplitudes can be determined by transforming the time-domain electrical signals to the frequency-domain via mathematical transformations (e.g., Fourier Transform) or other suitable techniques.

Additionally, the sleep states for which the detection algorithm can support predictions can include a Stage 1 sleep state, a Stage 2 sleep state, a Stage 3 sleep state, and a rapid eye movement (REM) sleep state. As an example, a frequency band for detecting Stage 1 sleep from EEG data can be defined to correspond to a particular type of wave and/or sleep stage. For example, the frequency band corresponding to the Stage 1 sleep state may be defined to extend between three to eight Hz. Thus, if amplitudes in the three to eight Hz band are between fifty to one-hundred μV (i.e., Theta waves), it may be inferred that the subject was in stage one sleep.

Additional characteristics of sleep states, such as sleep spindles and K-complexes, can be discerned via the detection algorithms to predict sleep states. For example, a high frequency band (e.g., a frequency band of around fifteen Hz) that, in the time domain, lasts for less than two seconds may be detected as a sleep spindle. Similarly, a low frequency band (e.g., a frequency band that extends between one and four Hz and amplitudes between one-hundred and two-hundred μV) (i.e., Delta waves) that, in the time-domain, lasts for about one second can be detected as a K-Complex. Therefore, if one or more portions of EEG data are detected as sleep spindles and are followed by or otherwise detected near one or more portions of EEG data detected as K-Complexes, it may be inferred that the subject was in Stage 2 sleep.

In another example, frequency bands extending between one to four Hz can be detected for significantly longer than two seconds (e.g., for twenty minutes), and from this, it may be predicted that the subject was in stage 3 sleep. Stage 3 sleep may also be referred to as slow-wave or delta sleep. Moreover, for the frequency bands that extend between about thirteen and about sixty hertz (Hz) and for amplitudes of at least about thirty μV (i.e., Beta waves), it may be predicted that the subject was in REM sleep. However, Beta waves may also be detected during the wake sleep state. Therefore, additional physiological data, physical or biological indicators, or other suitable data can be obtained and identified within the detection algorithms to differentiate between the REM sleep state and the wake sleep state. For example, EMG data may be obtained, and a detection algorithm may detect phasic events (e.g. rapid eye movements and twitches of the limbs) or tonic phenomena (e.g. loss of tone in antigravity muscles) from the EMG data, both of which can be indicative of REM sleep. The detection of phasic events or tonic phenomena can be compared or combined with the neural-signal data to distinguish the REM sleep state from the wake sleep state or another sleep state.

Illustrative examples are given to introduce the reader to the general subject matter discussed herein and are not intended to limit the scope of the disclosed concepts. The following sections describe various additional features and examples with reference to the drawings in which like numerals indicate like elements, and directional descriptions are used to describe the illustrative aspects, but, like the illustrative aspects, should not be used to limit the present disclosure.

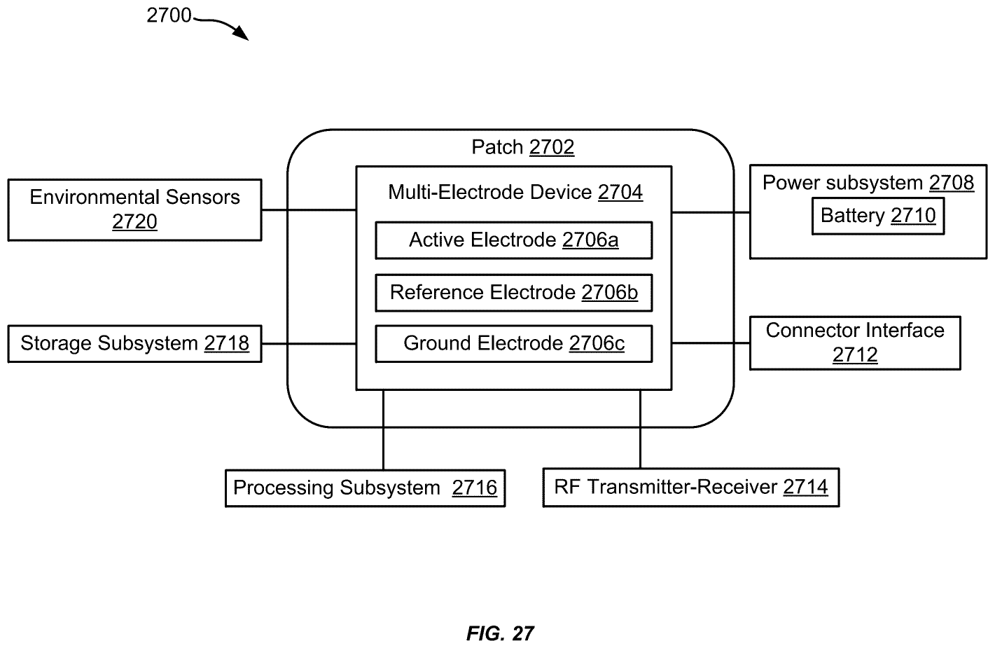

FIG. 27 is a block diagram of an example of a system 2700 for acquiring physiological data according to one example of the present disclosure. The system 2700 can include a multi-electrode device 2704, which can have one or more active electrodes 2706a for collecting active signals and one or more reference electrodes 2706b, which can collect corresponding reference signals. Additionally, the multi-electrode device 2704 may include a ground electrode 2706c. The electrodes 2706a-c can be fixed in location within a device (e.g., patch 2702) or movable (e.g., tethered to a device). The system 2700 can further include a processing subsystem 2716, a storage subsystem 2718, a (radiofrequency) RF transmitter-receiver 2714, a connector interface 2712, a power subsystem 2708, and environmental sensors 2720, each of which can be communicatively coupled to or part of the multi-electrode device 2704.

The processing subsystem 2716 can be implemented as one or more integrated circuits, e.g., one or more single-core or multi-core microprocessors or microcontrollers, examples of which are known in the art. The processing subsystem 2716 can control the operation of multi-electrode device 2704 by executing a variety of programs in response to program code and may maintain multiple concurrently executing programs or processes. For example, the processing subsystem 2716 may execute code that can control collection, analysis, application and/or transmission of physiological data (e.g. electroencephalogram (EEG) data, electromyography (EMG) data, etc.). Some or all of the program code can be stored in the processing subsystem 2716 or the program code can be stored in storage media such as the storage subsystem 2718. Additionally, the processing subsystem 2716 may cause signals detected by the electrodes 2706a-c of the multi-electrode device 2704 to be amplified, filtered, or a combination thereof and may further store the signals along with recording details (e.g., a recording time or a user identifier). In some examples, the processing subsystem 2716 can analyze the physiological data or signals to detect physiological correspondences. For example, the recorded signals can reveal frequency properties that correspond to sleep stages.

Additionally, the storage subsystem 2718 can be implemented using, for example, magnetic storage media, flash memory, other semiconductor memory (e.g., DRAM, SRAM), or any other non-transitory storage medium, or a combination of media, and can include volatile and/or non-volatile media. In some examples, the storage subsystem 2718 can store physiological data, information (e.g., identifying information or medical-history information) about a subject, or analysis variables (e.g., frequencies, amplitudes, etc.) obtained from the physiological data. The storage subsystem 2718 can also store one or more programs that can be executed by the processing subsystem 2716. The one or more programs may initiate or otherwise control collection, analysis, or transmission of the physiological data.

The RF transmitter-receiver 2714 can enable the multi-electrode device 2704 to communicate wirelessly with various interface devices, such as a phone, tablet, laptop, etc. The RF transmitter-receiver 2714 can include a combination of hardware components including, for example, driver circuits, antennas, modulators, demodulators, encoders, decoders, other suitable analog and/or digital signal processing circuits and can also include software components. Various wireless communication protocols can be implemented via the RF transmitter-receiver 2714 using the software components and associated hardware. RF transceiver components of the RF transmitter-receiver 2714 can include an antenna and supporting circuitry to enable data communication over a wireless medium, such as Wi-Fi, Bluetooth®, or other suitable mediums for wireless data communication.

The connector interface 2712 can enable the multi-electrode device 2704 to communicate with various interface devices via a wired communication path, e.g., using Universal Serial Bus (USB), universal asynchronous receiver/transmitter (UART), or other protocols for wired data communication. In some examples, the connector interface 2712 can provide a power port for allowing the multi-electrode device 2704 to receive power. The connector interface 2712 may also provide connections to transmit or receive the physiological data. For example, the physiological data can be transmitted to or from another device, such as another multi-electrode device, in analog or digital formats.

The environmental sensors 2720 can include various electronic, mechanical, electromechanical, optical, or other devices that provide information related to external conditions around the multi-electrode device 2704 or with respect to the subject. Any type and combination of the environmental sensors 2720 can be used. For example, an accelerometer can be used to estimate whether a user is or is trying to sleep or otherwise estimate an activity state. In another example, an electrooculogram sensor can be used to detect eye-movement to assist in identifying a rapid eye movement (REM) sleep stage.

Additionally, the power subsystem 2708 can provide power and power management capabilities for the multi-electrode device 2704. For example, the power subsystem 2708 can include a battery 2710 and associated circuitry to distribute power from battery 440 to other components of the system 2700 that may require electrical power.

It will be appreciated that system 2700 is illustrative and that variations and modifications are possible. In an example, the processing subsystem 2716 can execute code from the storage subsystem 2718 for analyzing sleep states based on EEG data and predicting a rick-level metric based on the analysis, where the risk-level metric can be a likelihood that a subject has a TBI. Thus, the system 2700 may further include a user interface to enable a user to directly interact with the system 2700 to, for example, receive the risk-level metric. The risk-level metric may be displayed at the user interface as a percentage or another suitable format. For example, the risk-level metric may be output as a color corresponding to a severity of the risk (i.e., the likelihood). Thus, for example, the severity of the risk can be high, moderate, or low and corresponding colors output can be red, yellow, or green. Further, while the system 2700 is described with reference to particular blocks, it is to be understood that these blocks are defined for convenience of description and are not intended to imply a particular physical arrangement of component parts.

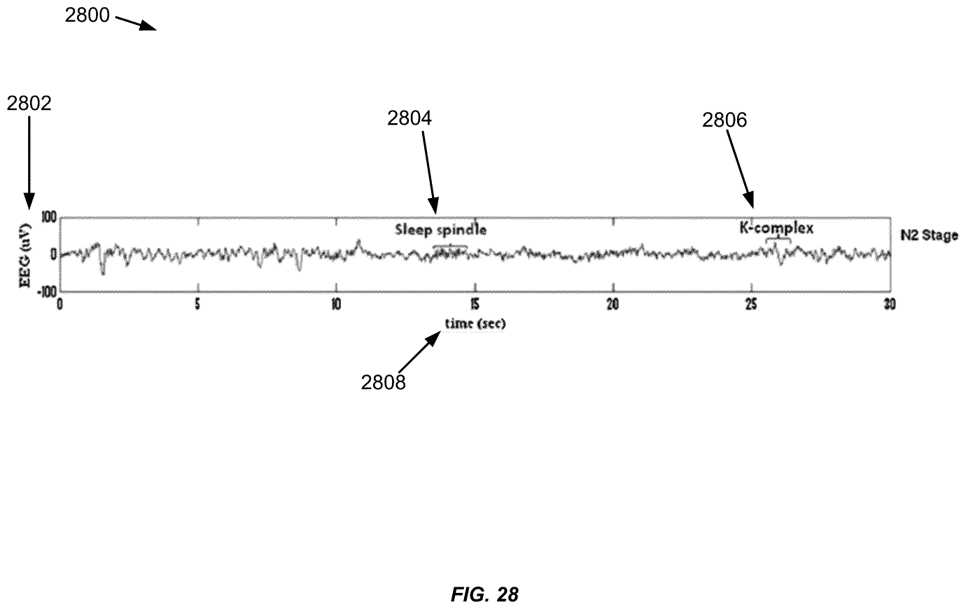

FIG. 28 is an example of a graph 2800 for predicting Stage 2 sleep according to one example of the present disclosure. The graph 2800 can include a typical EEG signal for a subject predicted to be in a Stage 2 sleep state. The graph 2800 can include amplitudes 2802 of the EEG signal in micro-volts on the y-axis and can include time 2808 in seconds on the x-axis. Thus, the graph 2800 can be a visual representation of electrical activity of a part of a brain of a subject over a thirty second timeframe during the Stage 2 sleep state. It can be predicted that the graph 2800 is representative of the Stage 2 sleep stage based on the presence of sleep spindles (e.g., sleep spindle 2804) and K-complexes (e.g., K-complex 2806).

The K-complexes and the sleep spindles can occur in any non-REM sleep stage (i.e., Stage 1, Stage 2, Stage 3), but are most prevalent in Stage 2. For example, during Stage 2 sleep, there can be between one and three K-complexes per minute, and each of the K-complexes may be associated with a preceding sleep spindle. Both K-complexes and sleep spindles tend to have durations between 0.5 and 2 seconds. Additionally, as depicted, K-complexes can have a first positive voltage peak, followed by a large negative complex, and finally followed by a second positive voltage peak. The K-complexes can be defined as a biphasic wave with a low frequency band (e.g., a frequency band that extends between one and four Hz). In contrast, the sleep spindles, such as the sleep spindle 2804, can be defined as brief, powerful bursts of high frequency (e.g., 11-15 Hz) activity.

In some examples, predicting Stage 2 sleep based on the EEG signal shown in graph 2800 can include implementing a detection algorithm. The detection algorithm can include deriving features of the EEG signal from the graph 2800 and detecting sleep spindles 2804, K-complexes 2806, or a combination thereof based on the features. For example, a sliding window of a first amount of time (e.g., 0.25, 0.5, or 1 second) with an overlap of a second amount of time (e.g., 0.1, 0.4, or 0.6 seconds) can be used to segment the EEG signal. Then, Short-time Fourier Transform (STFT) or another suitable mathematical technique can be applied to acquire time-frequency information about each segment of the EEG signal. Additionally, a fractional dimension (FD) technique or another suitable technique can be used to derive features (e.g., energy, power, etc.) based on the time-frequency information of each segment. Finally, a classification algorithm or another suitable type of machine-learning algorithm can be trained to classify the segments as, for example, sleep spindle, K-complex, or neither, based on the features. In this way, portions of the EEG signal associated with the sleep spindles and the K-complexes can be detected. The detection of sleep spindles and K-complexes can indicate that the EEG signal is associated with Stage 2 sleep.

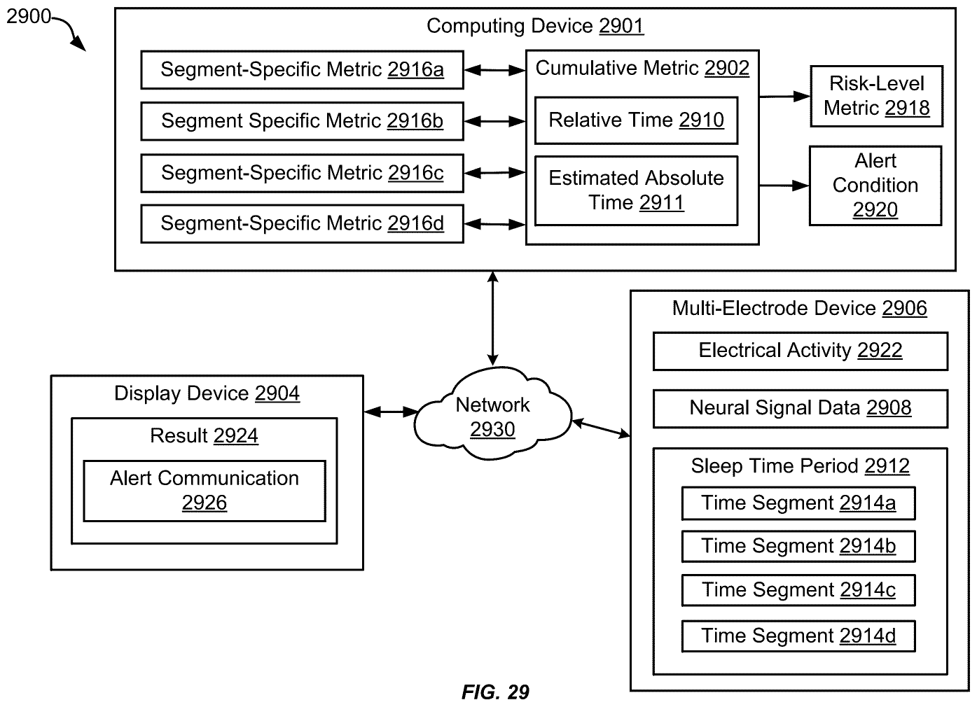

FIG. 29 is a block diagram of an example of a system 2900 for predicting the presence of a traumatic brain injury (TBI) based on metrics associated with sleep states according to one example of the present disclosure. The system 2900 can include a computing device 2901, which can be communicatively coupled with a display device 2904 and a multi-electrode device 2906. The computing device 2901 may communicate with the display device 2904 and the multi-electrode device 2906 via a network 2930, such as a local area network (LAN) or the internet. Additionally, the system 2900 can collect physiological data via the multi-electrode device 2906. The multi-electrode device 2906 can correspond to the multi-electrode device 2704 of FIG. 27. The physiological data can include neural signal data 2908 (i.e., electroencephalogram (EEG) data), electromyogram (EMG) data, electrocardiogram (ECG) data, electrooculogram (EOG) data, or other suitable physiological data.

In some examples, the computing device 2901 can access the physiological data. For example, the computing device 2901 may access the neural signal data 2908 collected via the multi-electrode device 2906. The neural signal data 2908 can be indicative of electrical activity 2922 from a part of the brain of a subject over any number of sleep time periods.

In a particular example, the neural-signal data 2908 can be indicative of electrical activity 2922 from a part of a brain of a subject over sleep time period 2912, which can be a twenty-minute portion of a night of sleep. The sleep time period 2912 can be further split, by the computing device 2901, into time segments 2914a-d (i.e., epochs). The time segments 2914a-d can each be a predefined length of time (e.g., one, five, or ten minutes) and the computing device 2901 can predict segment-specific metrics 2916a-b for each of the time segments 2914a-d. To support the predictions of segment-specific metrics 2916a-d, detection algorithms can be configured in the time domain, the frequency domain, or the time-frequency domain to derive features (e.g., frequency bands, amplitudes, intensities, time periods, etc.) of the neural signal data 2908 for each of the time segments 2914a-d. Then, the features can be used to predict the segment-specific metrics 2916a-d.

In some examples, the segment-specific metrics 2916a-d can be predicted probabilities of the time-segments 2914a-d being a particular sleep stage. In the particular example, a first segment-specific metric 2916a can be a ninety percent likelihood that a first-time segment 2914a is representative of Stage 2 sleep. A second segment-specific metric 2916b can be an eighty-five percent likelihood that a second time segment 2914b is representative of Stage 2 sleep. A third segment-specific metric 2916c can be a fifty percent likelihood that a third time segment 2914c is representative of Stage 2 sleep. Finally, a fourth segment-specific metric 2916d can be a twenty percent likelihood that a fourth time segment 2914d is representative of Stage 2 sleep.

In some examples, the third time segment 2914c may be further analyzed in smaller time segments to predict whether any portion of the third time segment 2914c is associated with Stage 2. Additionally, in some examples, the sleep time period 2912 can be one of many sleep time periods spanning one or more sleep cycles or one or more nights of sleep for the subject. The sleep time periods can be any length of time and can be segmented into any number of time segments.

Additionally, the computing device 2901 can generate a cumulative metric 2902 based on the segment-specific metrics 2916a-b. For example, time segments associated with predicted probabilities of Stage 2 sleep above a threshold can be summed to generate the cumulative metric 2902. In the particular example, an estimated absolute time for Stage 2 sleep can be determined based on the segment-specific metrics 2916a-d for the sleep time period 2912. Then, a cumulative metric 2902 may be generated by summing the estimated absolute time for sleep time period 2912 and additional estimated absolute times for Stage 2 sleep determined for additional sleep time periods. The sleep time period 2912 and the additional sleep time periods may span a single night of sleep for the subject. Thus, the cumulative metric 2902 can be an estimate absolute time for which it is predicted that the subject was in Stage 2 sleep over the night of sleep. For example, the sleep time periods can span six hours and the cumulative metric 2902 can be ninety minutes. In another example, the cumulative metric 2902 may be converted to a relative time. Thus, cumulative metric 2902 can be twenty-five percent.

The computing device 2901 can further generate a risk-level metric 2918 based on the cumulative metric 2902. The risk-level metric 2918 can be a likelihood that the subject has a TBI. In some examples, artificial intelligence techniques can be implemented to generate the risk-level metric 2918. The artificial-intelligence techniques may include using one or more models or rules to generate subject-specific predictions based on the cumulative metric 2902. In some examples, the presence of a TBI can be associated with a reduction in Stage 2 sleep, an increase in Stage 3 sleep, a combination thereof, or other suitable changes in typical sleep patterns. Thus, the rules can be defined to predict deprivation of Stage 2 sleep, excessive Stage 3 sleep, and/or TBI likelihood based on the cumulative metric 2902.

In some examples, the computing device 2901 may train a machine learning algorithm to predict the risk-level metric 2918 by inputting historical neural signal data with an indication of whether the data relates to a healthy subject or a subject with a TBI. For example, the historical neural signal data can include previous neural signal data associated with sleep for the subject, neural signal data collected for a healthy population, neural signal data collected for a population of subjects diagnosed with TBIs, or another suitable population for which neural signal data can be analyzed and compared to the neural signal data 2908 for the subject.

Additionally, in some examples, threshold amounts of time or other suitable values associated with Stage 2 sleep can be predefined based on age group or another suitable feature of subjects. The threshold amounts of time for Stage 2 sleep may be a gradient such that each of the multiple threshold amounts of Stage 2 sleep correspond to a different levels of risk. Additionally, a machine learning algorithm or other suitable AI technique can be implemented to predict the threshold amounts based on sleep data for subjects in the age group and may further be used to predict corresponding risk-level metrics for the threshold amounts. For example, for an age group of thirty to fifty years old, the threshold amounts of Stage 2 sleep can be relative times of forty percent, thirty percent, and twenty percent. The relative times can correspond to risk-level metrics of about 70%, 80%, and 90%. Thus, for the particular example, if the subject is thirty-five years old and the cumulative metric 2902 for Stage 2 sleep over the sleep time period 2912 is twenty-five percent, the computing device 2801 may predict a risk-level metric of eighty percent. Thus, the likelihood that the subject has a traumatic brain injury can be eighty percent.

In response to generating the cumulative metric 2902 and/or the risk-level metric 2918, the computing device 2901 can output, to a display device 2904. a result 2924. The result 2924 can be a value for the risk-level metric 2918 or otherwise be representative of the risk-level metric 2918. In some examples, the result 2924 can be output to the display device 2904 in response to the computing device 2901 determining that an alert condition 2926 is satisfied. For example, the alert condition 2926 can be a threshold likelihood. Thus, the result 2924 can be output in response to the risk-level metric 2918 exceeding the threshold likelihood. Additionally, outputting the result 2924 can include transmitting an alert communication 2928 to a third-party system associated with monitoring the subject.

In this way, detection and diagnosis of TBIs can be improved. In particular, by predicting metrics associated with a sleep stage and by predicting, based on a cumulation of the metrics, the risk-level metric 2918, an accuracy of diagnosing TBIs can be improved. Additionally, monitoring the subject to generate the risk-level metric 2918 and outputting the result 2924 can increase efficiency of diagnosis, which can thereby facilitate efficient treatment of TBIs.

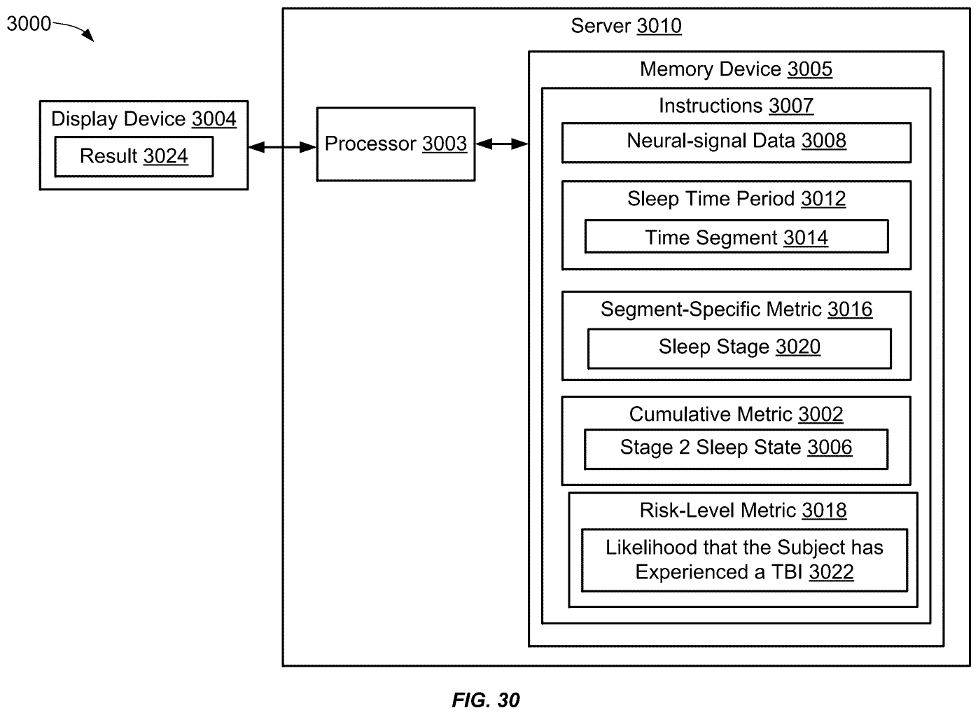

FIG. 30 is a block diagram of an example of a computing system 3000 for predicting the presence of a traumatic brain injury (TBI) based on metrics associated with sleep states according to one example of the present disclosure. The computing system 3000 includes a processor 3003 that is communicatively coupled to a memory device 3005. In some examples, the processor 3003 and the memory device 3005 can be part of the same computing device, such as the server 3010. In other examples, the processor 3003 and the memory device 3005 can be distributed from (e.g., remote to) one another.

The processor 3003 can include one processor or multiple processors. Non-limiting examples of the processor 3003 include a Field-Programmable Gate Array (FPGA), an application-specific integrated circuit (ASIC), or a microprocessor. The processor 3003 can execute instructions 3007 stored in the memory device 3005 to perform operations. The instructions 3007 may include processor-specific instructions generated by a compiler or an interpreter from code written in any suitable computer-programming language, such as C, C++, C#, Java, or Python.

The memory device 3005 can include one memory or multiple memories. The memory device 3005 can be volatile or non-volatile. Non-volatile memory includes any type of memory that retains stored information when powered off. Examples of the memory device 3005 include electrically erasable and programmable read-only memory (EEPROM) or flash memory. At least some of the memory device 3005 can include a non-transitory computer-readable medium from which the processor 3003 can read instructions 3007. A non-transitory computer-readable medium can include electronic, optical, magnetic, or other storage devices capable of providing the processor 3003 with computer-readable instructions or other program code. Examples of a non-transitory computer-readable medium can include a magnetic disk, a memory chip, ROM, random-access memory (RAM), an ASIC, a configured processor, and optical storage.

The processor 3003 can execute the instructions 3007 to perform operations. For example, the processor 3003 can access neural-signal data 3008 indicative of electrical activity from a part of the brain of a subject over one or more sleep time periods 3012. The processor 3003 can also predict, for each of one or more time segments 3014 in the one or more sleep time periods 3012, a segment-specific metric 3016 associated with a sleep stage 3020. The processor 3003 can further generate a cumulative metric 3002 based on the segment-specific metric 3016. The cumulative metric 3002 can correspond to an estimated absolute or relative time during which the subject was in a Stage 2 sleep state 3006. Additionally, the processor 3003 can generate, based on the cumulative metric 3002, a risk-level metric 3018 for the subject. The risk-level metric 3018 can represent a likelihood that the subject has a TBI 3022. Moreover, the processor 3003 can output a result 3024 that is based on or that represents the cumulative metric 3002. For example, the processor 3003 can output the result 3024 to a display device 3004.

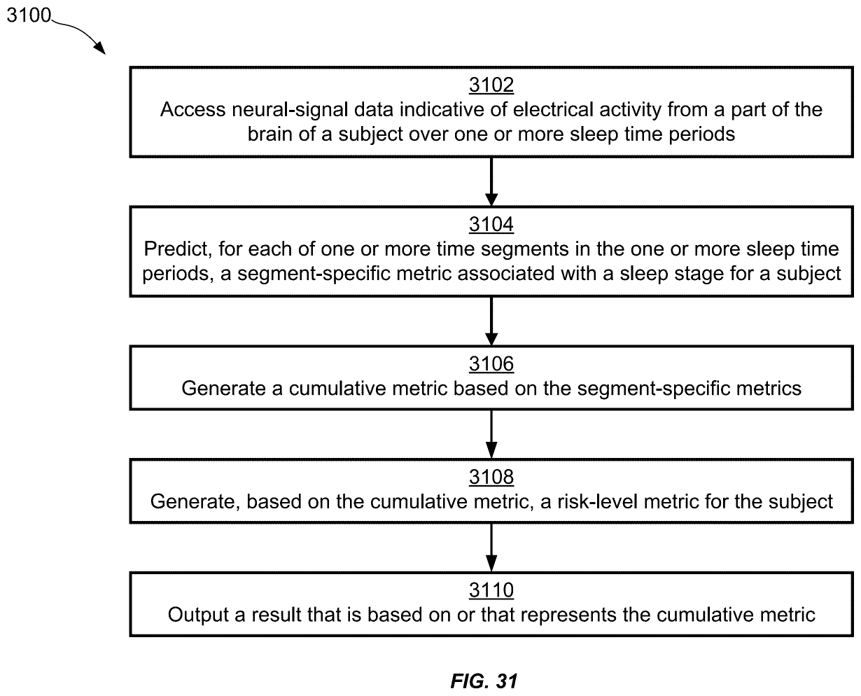

FIG. 31 is a flowchart of a process 3100 for predicting the presence of a traumatic brain injury based on metrics associated with sleep states according to one example of the present disclosure. In some examples, a processor 3003 can implement some or all of the steps shown in FIG. 31. Other examples can include more steps, fewer steps, different steps, or a different order of the steps than is shown in FIG. 31. The steps of FIG. 31 are discussed below with reference to the components discussed above in relation to FIGS. 29 and 30.

At block 3102, the processor 3003 can access neural-signal data 2908 indicative of electrical activity 2922 from a part of the brain of a subject over one or more sleep time periods 2912. The neural-signal data 2908 can be received or accessed from a multi-electrode device 2906. The neural-signal data 2908 can be electroencephalography (EEG) data. Additionally, the sleep time periods 2912 can correspond to a night of sleep (e.g., a six hour period of sleep), to multiple nights of sleep, or to a portion of the night of sleep (e.g., a sleep cycle).

At block 3104, the processor 3003 can predict, for each of one or more time segments 2914a-d in the one or more sleep time periods 2912, a segment-specific metric 2916a-d associated with a sleep stage for a subject. The sleep stage can be Stage 1, Stage 2, Stage 3, or REM. To predict the segment-specific metrics 2916a-d, the processor 3003 may perform at least one Fourier transform on the neural signal data 2908 of the time-segments 2914a-d. In this way, the neural signal data 2908 can be analyzed in the frequency domain to determine whether frequency bands, amplitudes, or other suitable frequency domain features of the neural signal data 2908 for each of the time segments 2914a-d is consistent with a particular sleep stage. Thus, in some examples, the segment-specific metrics 2916a-d can identify whether a time segment is associated with the particular sleep stage. For example, the segment-specific metrics 2916a-d can be predicted probabilities that the time segments 2914a-d are, for example, associated with Stage 3 sleep.

At block 3106, the processor 3003 can generate a cumulative metric 2902 based on the segment-specific metrics 2916a-d. For example, a subset of the time segments 2914a-d identified by the segment-specific metrics 2916a-d as Stage 3 sleep can be summed to generate the cumulative metric 2902. In particular, if the segment-specific metrics 2916a-d are predicted probabilities, the subset of the time-segments 2914a-d with a predicted probability above a probability threshold can be summed to generate the cumulative metric 2902. Thus, the cumulative metric 2902 may be an estimated absolute time (i.e., 90 minutes, 120 minutes, etc.) or a relative time (40%, 45%, etc.) for which it is estimated that the subject was in, for example, a Stage 3 sleep state of the sleep time periods 2912.

At block 3108, the processor 3003 can generate, based on the cumulative metric, a risk-level metric 2918 for the subject. The risk-level metric 2918 can represent a likelihood that the subject has experienced a TBI. In some examples, artificial intelligence techniques can be implemented to generate the risk-level metric 2918. The artificial-intelligence techniques may include using models or rules to generate subject-specific predictions based on the cumulative metric 2902. In some examples, the presence of a TBI can be associated with a reduction in Stage 2 sleep, an increase in Stage 3 sleep, a combination thereof, or other suitable changes in typical sleep patterns. Thus, the rules can be defined to predict deprivation of Stage 2 sleep, excessive Stage 3 sleep, and/or TBI likelihood based on the cumulative metric 2902.

At block 3110, the processor 3003 can output a result that is based on or that represents the cumulative metric 2902. The result 2924 can be a value of the cumulative metric 2902, a value for the risk-level metric 2918, or otherwise be representative of the cumulative metric 2902 and the risk-level metric 2918. In some examples, the processor 3003 may an alert condition 2926 is satisfied, and, in response, the result 2924 can be output to the display device 2904. For example, the alert condition 2926 can be a threshold, such as a threshold estimated absolute time for Stage 3 sleep. Thus, the result 2924 can be output in response to the cumulative metric 2902 exceeding the threshold. Additionally, outputting the result 2924 can include transmitting an alert communication 2928 to a third-party system associated with monitoring the subject.

### XI. General Considerations

Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provide a result conditioned on one or more inputs. Suitable computing devices include multi-purpose microprocessor-based computer systems accessing stored software that programs or configures the computing system from a general purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Embodiments of the methods disclosed herein may be performed in the operation of such computing devices. The order of the blocks presented in the examples above can be varied—for example, blocks can be re-ordered, combined, and/or broken into sub-blocks.

Certain blocks or processes can be performed in parallel.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

While the present subject matter has been described in detail with respect to specific embodiments thereof, it will be appreciated that those skilled in the art, upon attaining an understanding of the foregoing, may readily produce alterations to, variations of, and equivalents to such embodiments. Accordingly, it should be understood that the present disclosure has been presented for purposes of example rather than limitation, and does not preclude the inclusion of such modifications, variations, and/or additions to the present subject matter as would be readily apparent to one of ordinary skill in the art.

What is claimed is:

1. A method comprising:
accessing biological-signal data that was collected by a biological-signal data acquisition assembly that comprises a housing having one or more clusters of electrodes, wherein each cluster of the one or more clusters of electrodes comprises at least an active electrode;
identifying, based on the biological-signal data, a first signal that represents a first intent to move a first portion of a body of the subject, wherein the first signal is generated before a second signal, and wherein the second signal represents a second intent to move a second portion of the body of the subject;
translating the first signal to identify a first operation to be performed by a computing device; and
outputting first instructions to perform the first operation.

1. The method of claim 1, wherein the biological-signal data includes electroencephalography (EEG) data, and wherein the first signal is generated from a left hemisphere of a brain of the subject and the second signal is generated from a right hemisphere of the brain.

2. The method of claim 1, wherein the biological-signal data includes electromyography (EMG) data, and wherein the first portion is a left limb of the subject and the second portion is a right limb of the subject.

3. The method of claim 1, wherein the first operation includes performing one or more functions associated with a graphical user interface of the computing device, and wherein the first operation includes:
moving a cursor displayed on the graphical user interface from a first location to a second location.

4. The method of claim 1, wherein the first operation includes performing one or more functions associated with a graphical user interface of the computing device, and wherein the first operation includes:
inputting text onto the graphical user interface.

5. The method of claim 5, further comprising applying one or more machine-learning models to the inputted text to predict additional text to be inputted onto the graphical user interface.

6. The method of claim 1, wherein the first operation includes performing one or more functions associated with a graphical user interface of the computing device, and wherein the first operation includes inputting one or more images or icons on the graphical user interface.

7. The method of claim 1, wherein the first operation includes launching an application stored in the computing device or executing one or more commands associated with the application.

8. The method of claim 1, wherein the first operation includes:
selecting a first interface element over a second interface element of an intent-communication interface, wherein the first interface element is associated with a first interface-operation data and a second interface element is associated with a second interface-operation data;
identifying a second operation to be performed by the computing device by accessing the first interface-operation data of the selected first interface element; and
outputting second instructions to perform the second operation.

9. The method of claim 9, wherein the intent-communication interface is a tree that includes a root interface element connected to the first interface element and the second interface element.

10. The method of claim 9, further comprising:
accessing additional biological-signal data that was collected by the biological-signal data acquisition assembly at another time point;
identifying, based on the additional biological-signal data, a third signal that represents a third intent to move the second portion of a body of the subject, wherein the third signal is generated before a fourth signal, and wherein the fourth signal represents a fourth intent to move the first portion of the body of the subject;
translating the third signal to identify a third operation to be performed by a computing device;
selecting, based on the third operation, a third interface element over a fourth interface element of the intent-communication interface, wherein the third interface element and the fourth interface element are connected to the first interface element, and wherein the third interface element is associated with a third interface-operation data and a fourth interface element is associated with a fourth interface-operation data; and
identifying a fourth operation to be performed by the computing device by accessing the third interface-operation data of the selected third interface element; and
outputting third instructions to perform the fourth operation.

11. The method of claim 11, wherein the fourth operation includes inputting one or more alphanumerical characters on a graphical user interface of the computing device.

12. The method of claim 1, wherein the computing device is an augmented reality or virtual reality device, and wherein the first operation includes performing one or more operations associated with the augmented reality or virtual reality device.

13. The method of claim 1, wherein the computing device includes one or more robotic components, and wherein the first operation includes controlling the one or more robotic components.

15. A computer-implemented method comprising:
accessing neural-signal data indicative of electrical activity from a part of the brain of a subject over one or more sleep time periods;
predicting, for each of one or more time segments in the one or more sleep time periods, a segment-specific metric associated with a sleep stage;
generating a cumulative metric based on the segment-specific metrics, wherein the cumulative metric corresponds to an estimated absolute or relative time during which the subject was in a Stage 2 sleep stage;
generating, based on the cumulative metric, a risk-level metric for the subject, wherein the risk-level metric represents a likelihood that the subject has a traumatic brain injury; and
outputting a result that is based on or that represents the cumulative metric.

16. The computer-implemented method of claim 15, wherein predicting the segment-specific metric includes performing at least one Fourier transform on the neural signal data in the segment.

17. The computer-implemented method of claim 15, further comprising:
determining that an alert condition is satisfied based on the cumulative metric, wherein the result is output in response to determining that the alert condition is satisfied.

18. The computer-implemented method of claim 15, wherein outputting the result includes transmitting an alert communication to a third-party system associated with monitoring the subject.

19. The computer-implemented method of claim 15, wherein the neural-signal data includes electroencephalography data.

20. The computer-implemented method of claim 15, wherein the segment-specific metric identifies a predicted sleep stage.

21. The computer-implemented method of claim 15, wherein the segment-specific metric identifies a predicted probability of the subject being in the Stage 2 sleep stage.
